(19) United States
(12) Patent Application Publication
(10) Pub. No.: US 2023/0160018 A1
(43) Pub. Date: May 25, 2023

(54) CRYBETAB2 PREDICTS POOR BREAST CANCER OUTCOME AND SENSITIZES TUMORS TO NUCLEOLIN AND CDK INHIBITION

(71) Applicant: The Johns Hopkins University, Baltimore, MD (US)

(72) Inventors: Vanessa Ferreira Merino, Townson, MD (US); Martin Pomper, Baltimore, MD (US); Saraswati Sukumar, Columbia, MD (US)

(21) Appl. No.: 17/919,152

(22) PCT Filed: Apr. 16, 2021
(86) PCT No.: PCT/US2021/027843

§ 371 Date: Oct. 14, 2022

Related U.S. Application Data
(60) Provisional application No. 63/010,844, filed on Apr. 16, 2020.

(51) Int. Cl. C12Q 1/6886; A61K 31/517; A61K 31/519; A61K 31/551

(52) U.S. Cl. CPC C12Q 1/6886; A61K 31/517; A61K 31/519; A61K 31/551; C12Q 2600/158; C12Q 2600/118

(57) ABSTRACT

CKYPB2 induces EMT, sternness, protein synthesis and cell cycle progression through regulation of nucleolin, con-tributing to an increase in tumor growth and metastasis. CKYPB2 can be used as a prognostic marker in African American women with TNBC. CKYPB2 can further select patients with TNBC and ER positive tumors that will likely benefit from inhibitors of ribosomal RNA synthesis, CDK4 and nucleolin.

### RELATED APPLICATIONS

This application claims the benefit of priority under 35 U.S.C. § 119(e) to U.S. Provisional Application No. 63/010,844 filed Apr. 16, 2020, the entire contents of which is incorporated herein by reference in its entirety.

### STATEMENT OF GOVERNMENTAL INTEREST

This invention was made with government support under grant no. W81XWH-15-1-0017 and W81XWH-04-1-0595 awarded by the Department of Defense. The government has certain rights in the invention.

### BACKGROUND

Women of African American (AA) descent are 42% more likely to die of breast cancer than European American (EA) women (1-3). The higher incidence of estrogen receptor-negative (ER⁻) tumors with less favorable prognosis among AA women contributes to this survival health disparity (4-7); however, the greatest survival disparities occur within the good-prognosis hormone-receptor positive (HR⁺) subtypes (8).

Gene expression and DNA methylation profiles in both invasive tumors and benign breast tissues revealed differences between AA and EA women (9-13). Expression of phosphoserine phosphatase-like (PSPHL) and β-crystallin B2 (CRYβB2) was found to be up-regulated in tumors from AA individuals and this 2-gene signature correctly classifies AA and EA breast (2), prostate (14) and colorectal (15) tumors. Expression of CRYβB2 was found to be up-regulated in each breast cancer subtype (9,16) and normal breast (10) from AA women. Recently, CRYβB2 and the related pseudogene, CRYβB2P1, were shown to have a function in breast cancer, with CRYβB2 being involved in metastasis, chemoattraction, and tumorigenesis of highly aggressive triple-negative breast cancer (TNBC cells) (17) that lack expression of the estrogen, progesterone, or HER2 receptors.

There are two crystallin super-families: the small heat-shock proteins (α-crystallins) and the βγ-crystallins. The αB-crystallin is an oncoprotein expressed in basal-like breast carcinomas (8). CRYβB2 is among the major proteins of the vertebrate eye lens, and mutations in this gene are associated with cataract (19). CRYβB2 improved proliferation and survival of retinal ganglion (20), axons (21), and ovarian granulosa cells (22). Thus, it is possible that overexpression of CRYβB2 in normal breast cells may initiate proliferation and subsequently, if dysregulated, tumorigenesis.

As such, there is a need for identifying cancer biomarkers which can improve prognosis and therapy in women with breast cancer.

### SUMMARY

The present inventors show that CRYβB2 induced tumorigenesis of low malignant breast cells through induction of a mesenchymal/stem-like phenotype, cell cycle progression, regulation of protein translation, and recruitment of cancer-associated fibroblasts. The inventors also identified interacting partners with CRYβB2, such as nucleolin, and demonstrate its role in CRYβB2-stemness, tumorigenesis and metastasis. Furthermore, the inventors show that CRYβB2 protein is overexpressed in TNBC from AA women and is associated with worse disease outcome. Finally, the inventors targeted CRYβB2 tumors with different drugs used in the clinic, such as nucleolin, CDK4 and protein inhibitors, and have identified specific drug regimens that will improve outcomes for women having upregulated CRYβB2 expression in their tumors.

In accordance with an embodiment, the present invention provides a method for identifying a female subject as having a breast tumor which is responsive to CDK4 inhibitors comprising: a) testing a breast tumor tissue sample from the tumor of the subject for expression of CRYβB2 protein in the cells of the sample; b) comparing the level of expression in the sample of the subject with the level of expression in a reference breast tissue sample; and c) identifying the subject as having a breast tumor that is CRYβB2 positive and may likely respond to CDK4 inhibitors when the level of expression of CRYβB2 is greater than the level of expression of CRYβB2 in the reference sample.

In accordance with another embodiment, the present invention provides a method for identifying a female subject as having a breast tumor which is responsive to an anti-nucleolin agent comprising: a) testing a breast tumor tissue sample from the tumor of the subject for expression of CRYβB2 protein in the cells of the sample; b) comparing the level of expression in the sample with the level of expression in a reference breast tissue sample; and c) identifying the subject as having a breast tumor which is responsive to an anti-nucleolin agent when the level of expression of CRYβB2 protein in the cells of the sample is elevated compared to the level of expression of CRYβB2 protein in the cells of the reference breast tissue sample.

In accordance with an embodiment, the present invention provides a method for treating a female subject having a breast tumor which is responsive to CDK4 inhibitors comprising: a) testing a breast tumor tissue sample from the tumor of the subject for expression of CRYβB2 protein in the cells of the sample; b) comparing the level of expression in the sample of the subject with the level of expression in a reference breast tissue sample; c) identifying the subject as having a breast tumor that is CRYβB2 positive and may likely respond to CDK4 inhibitors when the level of expression of CRYβB2 is greater than the level of expression of CRYβB2 in the reference sample; and d) administering to the subject an effective amount of a CDK4 inhibitor.

In accordance with another embodiment, the present invention provides a method for treating a female subject having a breast tumor which is responsive to an anti-nucleolin agent comprising: a) testing a breast tumor tissue sample from the tumor of the subject for expression of CRYβB2 protein in the cells of the sample; b) comparing the level of expression in the sample with the level of expression in a reference breast tissue sample; c) identifying the subject as having a breast tumor which is responsive to an anti-nucleolin agent when the level of expression of CRYβB2 protein in the cells of the sample is elevated compared to the level of expression of CRYβB2 protein in the cells of the reference breast tissue sample and d) administering to the subject an effective amount of an anti-nucleolin agent.

### BRIEF DESCRIPTION OF THE DRAWINGS

FIGS. 1A-1H show that CRYβB2 is overexpressed in AA tumors and promotes tumorigenesis. FIG. 1A. Boxplot representation of CRYβB2 and CRYβB2P1 mRNA expression in breast tumors from Asian, European American (EA) and African American (AA). Wilcoxon rank sum test was used to calculate differences in expression.

FIG. 1B. Western blot determination of CRYβB2 protein expression in estrogen receptor (ER) negative tumors from AA and EA women (n=10 each). β-actin: loading control.

FIG. 1C. CRYβB2 mRNA expression in CD24 (differentiated) and CD44 (stem cell) populations isolated from AA and EA normal breast (n=6 each). Tumor volume and weight of MCF10AT1 (FIG. 1D) and DCIS.COM (FIG. 1E)—CRYβB2 and control xenografts (n=5 each). Standard error of mean (SEM) is reported. FIG. 1F. Bioluminescence imaging of MCF10AT1-CRYβB2 and control tumors and distant metastases to mammary gland, lung and bone. FIG. 1G. CRYβB2 IHC of lung and mammary gland. FIG. 1H. Image J quantification of lung area with metastases. Mann-Whitney test was performed. *p<0.05, **p<0.01 and ***p<0.001.

FIGS. 2A-2F demonstrate that CRYβB2 tumors present features of aggressive breast cancer. FIG. 2A. Morphological analysis of MCF10AT1-CRYβB2 xenografts following 16 weeks of growth and stained with hematoxylin and eosin (HE); IHC for: fibrillarin, CRYβB2, mouse alpha smooth muscle actin (α-SMA), and vimentin.

FIG. 2B. Western blot determination of epithelial and mesenchymal markers in DCIS.COM and MCF10AT1 tumors. β-actin: loading control. FIG. 2C. Mammosphere assay using MCF10AT1-CRYβB2 and control cells. FIG. 2D. ELDA software was used to calculate the cancer stem cell (CSC) frequency in limiting-dilution assay of MCF10AT1-CRYβB2 and control cells at week 3 of tumor growth. Different cell doses and tumor incidence are shown. FIG. 2E. Flow cytometry determination of CD44+/CD24− (CSC) and CD44+/CD24+/EpCAM+(differentiated) populations in MCF10AT1-CRYβB2 and control tumors and metastases within the distal mammary gland. Student's t-test was performed, and results are expressed as mean±SEM. FIG. 2F. Gene Set Variation Analysis (GSVA) scores of gene set analysis (GSEA) hallmark gene sets for MCF10AT1-CRYβB2 cells in comparison to vector. Representative pathways are shown. *p<0.05, **p<0.01 and ***p<0.001.

FIGS. 3A-3H show the CRYβB2 interactome and regulation of protein translation. FIG. 3A. Scheme of HuProt™ human proteome microarray—based discovery.

FIG. 3B. Identification of CRYβB2-associated proteins and representative images of binding of CRYβB2 antibody to nucleolin (NCL), PAIP1 and GRB2 in the presence of MCF10AT1-CRYβB2 lysate or 5% BSA control. FIG. 3C. Co-immunoprecipitation of CRYβB2 and its associated proteins, using total protein lysate (input) or immunoprecipitated complexes (IP) from MCF10AT1-vector and -CRYβB2 cells. FIG. 3D. Western blot determination of PAIP1 protein in MCF10AT1-vector and -CRYβB2 tumors. FIG. 3E. SUnSET measurement of protein synthesis in MCF10AT1 and DCIS.COM-CRYβB2 and control cells treated with homoharringtonine (HHT, 50 nM) for 48 h, followed by puromycin (1 μM) for 30 min. Protein synthesis was detected by immunoblotting with an anti-puromycin antibody. FIG. 3F. Proliferation assay and quantification of MCF10AT1-vector and -CRYβB2 cells treated with vehicle and or homoharringtonine (HHT, 10 nM) for 10 days. FIG. 3G. Mean tumor volume±SEM of 5 mice per group bearing MCF10AT1-CRYβB2 xenografts and treated for 5 weeks with vehicle (saline) or HHT (1 mg/kg, i.p.). FIG. 3H. Immunofluorescence staining of MCF10AT1-CRYβB2 cells with anti-CRYβB2-flag (red), anti-protein disulfide isomerase (PDI) (green) and Hoechst (blue). The merge of the fluorescent channels is shown (right). *p<0.05, **p<0.01 and ***p<0.001.

FIGS. 4A-4H demonstrate that CRYβB2 associates to nucleolin and regulates its pathway. FIG. 4A. Immunofluorescence staining of MCF10AT1-CRYβB2 cells with anti-CRYβB2 (red), anti-nucleolin (green) and Hoechst (blue). Western blot determination of CRYβB2, nucleolin (NCL) and downstream effectors protein levels in (FIG. 4B) MCF10A-BRCA1-KI, and (FIG. 4C) MCF10AT1 and DCIS.COM tumors overexpressing CRYβB2 and control plasmids (n=5-6 tumors each). FIG. 4D. Senescence beta-galactosidase staining of MCF10AT1-vector and -CRYβB2 tumors. FIG. 4E. Scheme of Western blot conclusions of CRYβB2-nucleolin pathway. Western Blot determination of nucleolin, CRYβB2 and downstream effectors protein expression in MCF10AT1-vector and -CRYβB2 cells vector and NCL knockout (KO) and CRYβB2 knockdown (KD) (FIG. 4F); TNBC (FIG. 4G) and estrogen receptor (ER)⁺ (FIG. 4H) cells. (*) Cell lines from AA patients. Direct correlation of protein expression, pearson correlation coefficient (r) and the p-value are shown. β-actin: loading control. *p<0.05, **p<0.01 and ***p<0.001.

FIGS. 5A-5I demonstrate that CRYβB2 tumorigenesis is mediated by nucleolin and targeted by nucleolin inhibition. Cell proliferation (FIG. 5A) and sphere assay (FIG. 5B) with MCF10AT1-CRYβB2 and control cells with or without nucleolin knockouts (NCL-KO #1 and #2). FIG. 5C. Tumor volume and weight±SEM of 5 mice per group injected with MCF10AT1-CRYβB2 and control cells overexpressing vector or NCL-KO. FIG. 5D. ImageJ quantification of their lung metastases. FIG. 5E. Tumor volume of 5 mice per group injected with MCF10AT1-CRYβB2 and control cells and treated with the nucleolin aptamer AS-1411 and control CRO (22 μg, i.p.). FIG. 5F. Direct correlation of AS-1411 IC50 and CRYβB2 protein expression in the triple negative breast cancer (TNBC) cell line HCC1806, determined by proliferation assay and Western blot, respectively. Pearson correlation coefficient (r) and the p-value are shown. FIG. 5G. Cell proliferation in HCC1806 cells vector and CRYβB2-KD in the presence of AS-1411 (1 μM) or vehicle control. Tumor volume (FIG. 5H) and ImageJ quantification of lung metastases (FIG. 5I) of 5 mice per group injected with HCC1806-CRYβB2-KD and control cells and treated with AS-1411 and CRO (22 μg, i.p.). *p<0.05, **p<0.01 and***p<0.001.

FIGS. 6A-6D demonstrate that CRYβB2 associates with poor TNBC outcome in AA women. FIG. 6A. Representative images of CRYβB2 IHC showing CRYβB2 negative and positive TNBC from AA women (n=102). FIG. 6B. Quantification of nucleolar CRYβB2 staining and correlation with nucleolar size (score 0-3). FIG. 6C. Distribution of tumors with nucleolar and nuclear CRYβB2 negative and positive stain among AA-TNBC patients. DF: Disease-free, NDF: Never disease-free and Met: Metastatic. FIG. 6D. Kaplan Meier curves for disease-free and overall survival among AA-patients (n=86) with TNBC according to the intensity of the staining of CRYβB2 in the nucleolus (positive: score 1-3; negative: score 0) and nucleus (positive: score 2-3; negative: score 0-1). Pearson correlation coefficient (r) and the p value are shown.

FIGS. 7A-7B demonstrate that CRYβB2 associates to phosphorylated Rb and p53 in tumor cells. Immunofluorescence staining of MCF10AT1-CRYβB2 cells with anti-ppRb (red) (FIG. 7A) or anti-p53 (green) (FIG. 7B) and anti-CRYβB2-flag (green/red) and Hoechst (blue). The merge of the fluorescent channels is shown (right).

FIGS. 8A-8E demonstrate that CRYβB2 activates CDK4/pRb pathway in premalignant and tumor cells. Western blot analysis of proteins involved in G1 to S phase cell cycle transition in: MCF10A cells knockout for p53 (p53-KO) and knockin for either p53-R248W (p53-KI) or BRCA1-185delAG (BRCA1-KI), and stably transfected with vector control (−) or CRYβB2 (+) (FIG. 8A); MCF10AT1 and DCIS.COM tumors overexpressing CRYβB2 and control plasmids (FIG. 8B); and MCF10AT1 cells expressing vector control and nucleolin-knockout (NCL-KO1 and 2) (FIG. 8C); TNBC cells expressing vector control and NCL-KO (FIG. 8D) and ER⁺ cell lines (FIG. 8E). Direct correlation of CRYβB2 expression with cell cycle proteins, pearson correlation coefficient (r) and the p value (p) are shown. β-actin: loading control.*p<0.05, **p<0.01 and ***p<0.001.

FIGS. 9A-9D demonstrate that CRYβB2 induces cell cycle progression and sensitivity to CDK4 inhibitors. FIG. 9A. Flow cytometry determination of cell cycle distribution in cells isolated from MCF10AT1-CRYβB2 and control xenografts and their distal mammary gland metastases. FIG. 9B. Tumor volume and weight±SEM of 5 mice per group containing MCF10AT1-CRYβB2 or control tumors and treated for 2 weeks (yellow bar) with vehicle or palbociclib (Palbo, 50 mg/kg, oral).*vehicle vs palbociclib and #vector vs CRYβB2. FIG. 9C. Direct correlation of palbociclib IC50 nM³¹ and CRYβB2 protein expression, determined by Western blot, in TNBC and ER⁺ cells. FIG. 9D. Scheme with the role of CRYβB2 in sensitization of tumors to inhibitors of nucleolin and CDK4. *p<0.05, **p<0.01 and ***p<0.001.

FIGS. 10A-10E demonstrate that CRYβB2 and ppRb expression are associated with poor TNBC outcome in AA women. Western blot analysis of CRYβB2 and CDK4 proteins in estrogen receptor negative (ER⁻) tumors (n=10) from AA and EA women (FIG. 10A); ImageJ quantification of CRYβB2 and CDK4 protein levels and their correlation in ER⁻ tumors (FIG. 10B). FIG. 10C. Representative images of phosphorylated pRb (ppRb) IHC showing ppRb negative and positive tumors using TNBC from AA women (n=102), pathologist quantification, score 0-3 (ppRb high (2-3) and ppRb low (1)) and distribution of ppRb negative (score 0) and positive tumors among AA-TNBC patients. Correlation of nucleolar CRYβB2 and nuclear ppRb stain in AA-TNBC patients. The size of the dots represent the number of patients within each score. The Pearson correlation coefficient (r) and the p value are shown. FIG. 10D. Kaplan Meier disease-free survival curves for AA-patients with TNBC (n=87) according to nuclear ppRb status (negative/positive). FIG. 10E. Kaplan Meier survival curves for patients with TNBC (AA, n=86; EA, n=3) according to ppRb and nucleolar CRYβB2 staining intensity. DF: Disease-free, NDF: Never disease-free and Met: Metastatic.

FIGS. 11A-11F demonstrate that CRYβB2 is induced in AA tumors and promotes tumorigenesis of low malignant breast cells. FIG. 11A. Boxplot displaying CRYβB2 and CRYβB2P1 mRNA expression, in the different breast cancer subtypes and in normal breast of Asian, African American (AA) and European American (EA) women. Total read counts for each sample from TCGA with alignments only in CRYβB2, CRYβB2P1 and in both are shown. FIG. 11B. Western blot determination of CRYβB2 expression in MCF10A, MCF10AT1 and DCIS.COM cells infected with lentivirus containing vector control or CRYβB2 plasmids. β-actin: loading control. MCF10A, MCF10AT1 and DCIS.COM cells expressing vector control or CRYβB2 were plated in 2D cultures and the colonies were stained with crystal violet (FIG. 11C) or in 3D low adhesion cultures in agar (FIG. 11D). Representative images (FIG. 11E) and bioluminescence (FIG. 11F) of MCF10AT1-vector and -CRYβB2 tumors and distal mammary gland metastasis.

FIGS. 12A-12L demonstrate that CRYβB2 tumors present features that predict worse prognosis. Image J quantification of mouse alpha smooth muscle actin (α-SMA) (FIG. 12A) and vimentin (FIG. 12B) in MCF10AT1-CRYβB2 and vector tumors and epithelial and mesenchymal markers in DCIS.COM tumors (FIG. 12C). FIG. 12D. Western blot determination, in duplicate, of epithelial and mesenchymal markers in MCF10AT1 tumors overexpressing vector and CRYβB2. β-actin: loading control. FIG. 12E. Representative images of the whole well and ImageJ quantification of tumor spheres by MCF10AT1 and DCIS.COM cells overexpressing vector and CRYβB2. FIG. 12F. The extreme limiting dilution analysis (ELDA) software was used to calculate the cancer stem cell (CSC) frequency in limiting-dilution assay of MCF10AT1-CRYβB2 and vector cells at week 3 and 4 of tumor growth. The different cell dose and tumor incidence by the number of mice injected are indicated on the table. FIG. 12G. Flow cytometry determination of EpCAM+ population in MCF10AT1-CRYβB2 and control tumors and metastases (Mets) within the distal mammary gland. FSC: forward side scatter. FIG. 12H. Volcano plot (Log 2 fold change (FC) vs −Log 10 false discovery rate (FDR)) and (Log 2 fold change (FC) vs −Log 10 p-value) of genes up- and down-regulated in MCF10AT1 and DCIS.COM-CRYβB2 cells, respectively in comparison to vector. The orange dots represent the genes that have at least a two-fold increase or decrease in gene expression and a FDR<0.05. The listed genes showed p<0.01 and p-value<0.05 for DCIS.COM cells. FIG. 12I. Quantitative RT-PCR of few genes identified in the array analysis as differentially expressed in MCF10AT1-CRYβB2 cells in comparison to vector. RPL39 was used as control. Student's t-test was performed, and the mean (±SEM) of triplicate results, are shown. (*) compared to vector. FIG. 12J. Heatmap depicting unsupervised hierarchical clustering of the top 10% differentially expressed genes in MCF10AT1-CRYβB2 cells in comparison to vector. Color scale indicates the log 2 expression values. FIG. 12K. Gene Set Variation Analysis (GSVA) scores of gene set analysis (GSEA) hallmark gene sets for MCF10AT1-CRYβB2 cells in comparison to vector. Representative pathways are shown. FIG. 12L. Western blot determination of endoplasmic reticulum (ER) stress—related proteins.*p<0.05, **p<0.01 and ***p<0.001.

FIGS. 13A-13C demonstrate the CRYβB2 interactome and its association to endoplasmic reticulum. FIG. 13A. HuProt™ human proteome microarray determination of CRYβB2—associated proteins using lysate of MCF10AT1-CRYβB2 cells and 5% BSA control. The binding of the CRYβB2 antibody was developed with a Cy5-labeled secondary antibody. Immunofluorescence staining of MCF10AT1-CRYβB2 cells with anti-CRYβB2-flag (red), Hoechst (blue) and green channel anti-protein disulfide isomerase (PDI) (FIG. 13B) and receptor-binding cancer antigen expressed on SiSo cells (RCAS1) (FIG. 13C). The merge of the fluorescent channels is shown (right).

FIGS. 14A-14C demonstrate that CRYβB2 binds to nucleolin and regulates its expression and function. FIG. 14A. Immunofluorescence staining of MCF10AT1-CRYβB2 and TNBC (HCC1806 and HCC1143) cells with anti-CRYβB2 or anti-CRYβB2-flag (red), anti-nucleolin (green) and Hoechst (blue). The merge of the fluorescent channels is shown (right). ImageJ quantification of Western blot analysis of CRYβB2, nucleolin and interacting proteins in MCF10A-BRCA1-KI (FIG. 14B) and MCF10AT1 and DCIS.COM-vector and -CRYβB2 tumors (C). β-actin: loading control.*p<0.05, **p<0.01 and ***p<0.001.

FIGS. 15A-15H demonstrate that CRYβB2 tumorigenesis is mediated by nucleolin. FIG. 15A. Western Blot determination of nucleolin in MCF10AT1-vector and -CRYβB2 cells following CRISPR/CAS9 depletion of nucleolin (knockout, KO #1 and #2). β-actin: loading control. ImageJ quantification of cell proliferation (FIG. 15B) and mammosphere assay (FIG. 15C) using MCF10AT1-vector and -CRYβB2 cells following nucleolin depletion. FIG. 15D. Representative tumor images of 5 mice per group injected with MCF10AT1-CRYβB2 and control cells overexpressing vector or nucleolin knockout (NCL-KO). HE stain (FIG. 15E) and ImageJ quantification (FIG. 15F) of lung metastases. TNBC cell proliferation following NCL-KO, AS-1411 and CRO treatment (FIG. 15G) and CRYβB2-KD (FIG. 15H). *p<0.05, **p<0.01 and ***p<0.001.

FIGS. 16A-16B demonstrate that CRYβB2 associates with poor TNBC outcome in AA women. FIG. 16A. Representative images of CRYβB2 IHC showing CRYβB2 staining in different cell compartments in TNBC from AA women (n=102). FIG. 16B. Kaplan Meier curves for disease-free and overall survival among AA-patients (n=86) with TNBC according to the intensity of the staining of CRYβB2 in the cytoplasm (positive: score 2-3; negative: score 0-1). Pearson correlation coefficient (r) and the p value are shown.

FIGS. 17A-17E demonstrate the Image J quantification of Western blot analysis of proteins involved in G1 to S phase cell cycle transition in: MCF10A cells knockin for BRCA1-185delAG (BRCA1-KI) (FIG. 17A); knockin for p53-R248W (p53-KI) and knockout for p53 (p53-KO) (FIG. 17B); MCF10AT1 and DCIS.COM tumors (FIG. 17C) stably transfected with vector control (−) or CRYβB2 (+); MCF10AT1 cells expressing vector control, NCL-KO1 or NCL-KO2 (FIG. 17D). Western Blot determination of cell cycle progression proteins in AA and EA TNBC cell lines (FIG. 17E) and direct correlation of CRYβB2 expression with cell cycle proteins. (*) AA lines. Pearson correlation coefficient (r) and p value (p) are shown. β-actin: loading control.

FIGS. 18A-18B demonstrate that CRYβB2 and ppRb expression are associated with poor TNBC outcome in AA women. Disease-free survival Kaplan Meier curves of AA-patients (n=86) with nuclear ppRb (FIG. 18A), and both nuclear ppRb and nucleolar CRYβB2 (FIG. 18B). *p<0.05, **p<0.01 and ***p<0.001.

FIG. 19 shows the Antibodies and conditions used for Western Blot (WB), Immunohistochemistry (IHC) and Immunofluorescence (IF).

### DETAILED DESCRIPTION

African American (AA) women with estrogen receptor positive and triple negative breast cancer (TNBC) show higher breast cancer mortality rates in comparison to European American (EA). The present inventors show that CRYβB2 is overexpressed exclusively in tumor initiating cells of normal breast from AA, suggesting a role of CRYβB2 in the early events of tumor formation, and recurrence following treatment. In line with this, CRYβB2 induced the growth of tumors with a single hit mutation in MAPK pathway (22). Characterization of the role of CRYβB2 in low-malignant cells revealed several features associated with an increase in malignancy which correlated with a worse disease outcome in patients (38). Tumors arising from low-malignant cells with CRYβB2 overexpression were less differentiated, with an increase in size of nuclei and nucleoli, in number of tumor-associated fibroblasts, EMT markers, progenitor/stem cell content and metastasis. Accordingly, the inventors show that CRYβB2 interacts with several proteins that regulate cell proliferation and invasion. Similarly, CRYβB2 was recently shown to increase genes associated with EMT in a TNBC xenograft model (17). CRYβB2 mutations lead to apoptosis in human lens epithelial cells due to activation of unfolded protein response (UPR) (39) in the lumen of endoplasmic reticulum (ER) (40). Gene expression array and pathway analysis revealed that CRYβB2 activated UPR and DNA repair pathways and decreased apoptosis pathways. Accordingly, CRYβB2-tumors showed a decrease in markers of DNA damage and apoptosis and an increase in makers of DNA repair. CRYβB2-tumors also increased ER stress sensors, possibly as a way to control unfolded proteins in the ER due to rapidly proliferative rates. In line with these observations, CRYβB2 was associated to ER, and binds to proteins that regulate translation and trafficking of proteins from ER to Golgi. CRYβB2 cells induced protein synthesis and CRYβB2—tumors are sensitive to the protein inhibitor homoharringtonine (HHT). HHT is approved for treatment of chronic myeloid leukemia (41) and target TNBC in preclinical studies (42). According to the regulation of protein synthesis by CRYβB2, the inventors observed that this gene binds to nucleolin and regulates its expression and function. Nucleolin is a multifunctional protein that is mainly localized in the nucleolus, where it regulates ribosome biogenesis and contributes to cell proliferation (26). Nucleolin maintains embryonic (28) and breast cancer (30) stem cells function. Nucleolin was also implicated in EMT (43) and migration/invasion of tumor cells (27). Accordingly, the inventors observed that nucleolin mediates the CRYβB2—increase of tumor growth, stemness and metastasis. Without being held to any particular theory, the inventors foresee that CRYβB2 may determine cancer cell sensitivity to anti-nucleolin therapies, including the nucleolin aptamer AS1411 (44) and possibly to inhibitors of ribosome RNA synthesis, such as CX-5461 (45).

In addition to regulation of ribosome production, the nucleolus regulates genome stability, cell-cycle control, cellular senescence and stress responses, driving cancer growth and proliferation (46). Dysregulation of the major cancer-related signaling pathways like Myc, RAS/RAF/ERK, PI3K/AKT/mTOR, p53, pRb and PTEN altered activity of the RNA Pol I and nucleolus function (46). Nucleolar size has been used as predictive and prognostic biomarker in chemotherapeutic treatment (47) and clinical outcomes (48).

In addition to regulation of protein synthesis, nucleolin induces malignancy by regulation of cell cycle. Similar to CRYβB2, nucleolin associates to pRb (31) and p53 (32). Nucleolin is involved in post-transcriptional inhibition of the p53 (32). We also observed a CRYβB2—mediated decrease of p53 protein. The inventors observed that CRYβB2—tumors activated nucleolin and CDK4/pRb pathway, resulting in expansion of tumor cells in the proliferative S phase of the cell cycle. These findings are in agreement with previous findings that CRYβB2 regulates CDK4 and cyclin D2 in ovarian cells (19). CRYβB2—tumors were sensitive to inhibition of CDK4 by palbociclib.

Importantly, palbociclib was ineffective in vector-control MCF10AT1 tumors, which lack CRYβB2, demonstrating the ability of CRYβB2 to sensitize breast tumor cells to CDK4-inhibitors.

The application of CDK4/6 inhibitors has been particularly focused on ER positive breast cancers where an improvement was observed in progression-free survival in patients with metastatic breast cancer (49). Due to frequent RB loss, TNBC patients are considered to be poor candidates for CDK—inhibition (50). However, certain subtypes of TNBC cells, like luminal androgen receptor (LAR) were highly sensitive to CDK4/6-inhibition, suggesting that TNBC proliferation may still involve the CDK complex (51). A phase I/II trial is testing the safety and effectiveness of palbociclib with bicalutamide, an anti-androgen, for the treatment of androgen receptor (AR)-positive TNBC (NCT02605486) (52). CRYβB2 expression also correlated with activation of CDK4/pRb pathway and response to palbociclib (37) in TNBC and ER positive cell lines. Accordingly, ER negative tumors from AA women showed higher expression and correlation of CRYβB2 and CDK4 proteins in comparison to tumors from EA women. Collectively, the inventors show that CRYβB2 can define a subgroup of patients with TNBC and ER positive tumors who are likely to respond to CDK4 inhibitors.

Similar to its interaction partners nucleolin and p53 (53), the inventors observed that CRYβB2 is a nucleocytoplasmic shuttling protein and localizes to different cell compartments in cell lines and patient samples. Proteins that shuttle between the cytoplasm and the nucleus play a crucial role as transport carriers and signal transduction regulators within cells, including cell cycle regulation (53). According to CRYβB2-regulation of nucleolin, the inventors observed that nucleolar CRYβB2 expression correlated with an increase in nucleolus size in TNBC from AA patients. Nucleolar, and to a lesser extend nuclear, CRYβB2 expression most effectively identify the AA patients that are less likely to survive with TNBC. The inventors observed high frequency of nucleolar CRYβB2 expression in metastatic lesions.

The RB gene is mutated/loss in 20% of basal-like tumors (50). Accordingly, the inventors found phospho pRb (ppRb) expression in 85% ( 87/102) of TNBC cases from AA women. Here the inventors showed for the first time that ppRb protein correlated with a worst TNBC outcome in AA women. Nucleolar CRYβB2 and ppRb expression were also correlated in TNBC patients and may likely predict response to CDK4 inhibitors. According to the role of CRYβB2 on activation of CDK4/pRb activation it was observed that only ppRb positive tumors with nucleolar CRYβB2 expression have a significant decrease in disease-free and overall survival.

In view of the foregoing, In accordance with an embodiment, the present invention provides a method for identifying a female subject as having a breast tumor which is responsive to CDK4 inhibitors comprising: a) testing a breast tumor tissue sample from the tumor of the subject for expression of CRYβB2 protein in the cells of the sample; b) comparing the level of expression in the sample of the subject with the level of expression in a reference breast tissue sample; and c) identifying the subject as having a breast tumor that is CRYβB2 positive and may likely respond to CDK4 inhibitors when the level of expression of CRYβB2 is greater than the level of expression of CRYβB2 in the reference sample.

As used herein, the term “female subject” includes female humans of any ethnicity or genetic background. Because CRYβB2 predicts activation of CDK4/P—Rb and therefore, a positive therapeutic response to CK4 inhibitors, any female subject that has elevated levels of CRYβB2 in their tumors compared to normalized reference breast tissue levels is a candidate for this treatment. The inventors have shown that frequently African or African-American women have elevated levels of CRYβB2 in their tumors, they are more likely to respond to the inventive methods. Similarly, it will be understood by those of ordinary skill in the art European or European-American women with elevated levels of CRBB2 in their tumors may also respond.

In accordance with an embodiment, the present invention provides a method for treating a female subject having a breast tumor which is responsive to CDK4 inhibitors comprising: a) testing a breast tumor tissue sample from the tumor of the subject for expression of CRYβB2 protein in the cells of the sample; b) comparing the level of expression in the sample of the subject with the level of expression in a reference breast tissue sample; c) identifying the subject as having a breast tumor that is CRYβB2 positive and may likely respond to CDK4 inhibitors when the level of expression of CRYβB2 is greater than the level of expression of CRYβB2 in the reference sample; and d) administering to the subject an effective amount of a CDK4 inhibitor.

The methods of the present invention are simple and quantitative, require very small amounts of tissue, provide an integrated readout of pathways active in the target tissues, and have the potential for automation. These inventive methods facilitate a more precise classification of patients based on activity of specific pathways in the target tissue. The inventive methods can be used as a molecular diagnostic to more precisely delineate disease subsets, and assist in selecting patients for therapy or for monitoring effectiveness.

The terms “sample,” “patient sample,” “biological sample,” and the like, encompass a variety of sample types obtained from a patient, individual, or subject and can be used in a diagnostic, prognostic or monitoring assay. The patient sample may be obtained from a healthy subject, a diseased patient including, for example, a patient having associated symptoms of breast cancer. Moreover, a sample obtained from a patient can be divided and only a portion may be used for diagnosis, prognosis or monitoring. Further, the sample, or a portion thereof, can be stored in Formalin-fixed, Paraffin-embedded (FFPE) samples under conditions to maintain sample for later analysis by immunohistochemistry (IHC). The definition specifically encompasses blood and other liquid samples of biological origin (including, but not limited to, peripheral blood, serum, plasma, urine, saliva, amniotic fluid, stool and synovial fluid), solid tissue samples such as a biopsy specimen or tissue cultures or cells derived therefrom and the progeny thereof.

In a specific embodiment, a sample comprises a breast cancer biopsy sample. In other embodiments, a sample comprises a blood or serum sample. The definition also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The terms “providing a sample” and “providing a biological (or patient) sample” are used interchangeably and mean to provide or obtain a biological sample for use in methods described in this invention. Most often, this will be done by removing a sample of cells from a patient such as a biopsy sample from a tumor, but can also be accomplished by using previously isolated cells (e.g., isolated by another person, at another time, and/or for another purpose), or by performing the methods of the invention in vivo. Archival tissues, having treatment or outcome history, can also be used.

A method of identifying a protein-associated with a disease or a pathological condition is also provided. The method comprises measuring a level of the protein in a sample that is different than the level of a control. In accordance with an embodiment, the protein detection may be performed by contacting the sample with an antibody against CRYβB2 and cell cycle proteins and develop using IHC or Western Blot assays.

The level of the CRYβB2 protein in the sample may also be compared to a control CRYβB2 negative tumor to determine whether the protein is overexpressed. The ability to identify proteins that are differentially expressed in pathological cells compared to a control can provide high-resolution, high-sensitivity datasets which may be used in the areas of diagnostics, prognostics, therapeutics, drug development, pharmacogenetics, biosensor development, and other related areas.

The expression level of a disease-associated protein, such as CRYβB2, provides information in a number of ways. For example, a differential expression of a disease-associated protein compared to a control may be used as a diagnostic that a patient will not present a good disease prognosis. Expression levels of a disease-associated protein may also be used to monitor the treatment and disease state of a patient. Furthermore, expression levels of a disease-associated protein may allow the screening of drug candidates for altering a particular expression profile or suppressing an expression profile associated with disease.

Levels of expression of CRYβB2 can also be measured by detecting the protein in the cells or nuclei of cells using antibodies or fragments thereof, which are conjugated with a detectable moiety or label. IHC staining was performed on 5-micron paraffin sections of the patient tumor using a DAKO EnVision System, HRP (DAB) Anti-Mouse (K4007) or Anti-Rabbit Kit (K4011), according to manufacturer's instructions (Agilent). The rabbit anti-CRYβB2 (#NBP2-13876, Novus Biologicals, 1:100) primary antibody was used following antigen retrieval with DAKO Target Antigen Retrieval (Tris/EDTA buffer, pH 9, #S2367) and incubation overnight at 4° C.

A “label” or a “detectable moiety” is a composition detectable by spectroscopic, photochemical, biochemical, immunochemical, chemical, or other physical means. For example, useful labels include³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into nucleic acids, proteins and antibodies at any position. Any method known in the art for conjugating the antibody to the label may be employed, e.g., using methods described in Hermanson, Bioconjugate Techniques 1996, Academic Press, Inc., San Diego.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab', F(ab').sub.2, Fab, Fv and rIgG. See also, Pierce Catalog and Handbook, 1994-1995 (Pierce Chemical Co., Rockford, Ill.). See also, e.g., Kuby, J., Immunology, 3.sup.rd Ed., W. H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An antibody immunologically reactive with a particular antigen, such as CRYβB2 can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain Each heavy and light chain contains a constant region and a variable region, (the regions are also known as “domains”). Light and heavy chain variable regions contain four framework” regions interrupted by three hypervariable regions, also called complementarity-determining regions (CDRs).

“Epitope” or “antigenic determinant” refers to a site on an antigen to which an antibody binds. Epitopes can be formed both from contiguous amino acids or noncontiguous amino acids juxtaposed by tertiary folding of a protein, such as CRYβB2. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance See, e.g., Epitope Mapping Protocols in Methods in Molecular Biology, Vol. 66, Glenn E. Morris, Ed (1996).

Antibodies can be used to detect CRYβB2 in the methods of the invention. The detection and/or quantification of CRYβB2 can be accomplished using any of a number of well recognized immunological binding assays. A general overview of the applicable technology can be found in Harlow & Lane, Antibodies: A Laboratory Manual (1988) and Harlow & Lane, Using Antibodies (1999). Other resources include see also Methods in Cell Biology: Antibodies in Cell Biology, volume 37 (Asai, ed. 1993); Basic and Clinical Immunology (Stites & Ten, eds., 7th ed. 1991, and Current Protocols in Immunology (Coligan, et al. Eds, John C. Wiley, 1999-present). Immunological binding assays can use either polyclonal or monoclonal antibodies.

Commonly used assays include noncompetitive assays (e.g., sandwich assays) and competitive assays. In competitive assays, the amount of CRYβB2 expression product present in the sample is measured indirectly by measuring the amount of a known, added (exogenous) expression product displaced (competed away) from an anti-expression product antibody by the unknown present in a sample. Commonly used assay formats include immunoblots, which are used to detect and quantify the presence of protein in a sample. Other assay formats include liposome immunoassays (LIA), which use liposomes designed to bind specific molecules (e.g., antibodies) and release encapsulated reagents or markers, which are then detected according to standard techniques (see Monroe et al., Amer. Clin. Prod. Rev. 5:34-41 (1986)).

Immunoassays also often use a labeling agent to specifically bind to and label the complex formed by the antibody and antigen. The labeling agent may itself be one of the moieties comprising the antibody/antigen complex. Thus, the labeling agent may be a labeled for CRYβB2 or a labeled anti-CRYβB2 antibody. Alternatively, the labeling agent may be a third moiety, such as a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the labeling agent. The labeling agent can be modified with a detectable moiety, such as biotin, to which another molecule can specifically bind, such as streptavidin. A variety of detectable moieties are well known to those skilled in the art.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent compounds (e.g., fluorescein isothiocyanate, Texas red, rhodamine, fluorescein, and the like), radiolabels, enzymes (e.g., horse radish peroxidase, alkaline phosphatase and others commonly used in an ELISA), streptavidin/biotin, and colorimetric labels such as colloidal gold or colored glass or plastic beads (e.g., polystyrene, polypropylene, latex, etc.). Chemiluminescent compounds may also be used. For a review of various labeling or signal producing systems that may be used, see U.S. Pat. No. 4,391,904.

In some embodiments, the detection of expression of CRYβB2 protein is in the nucleoli, nuclei and cytoplasm of the cells of the tumor sample.

As used herein, the term “anti-CDK4 inhibitors” means one or more agents which act at the G₁-to-S cell cycle checkpoint and inhibit cycle progression leading to cell cycle arrest. Examples of such inhibitors include, but are not limited to Palbociclib, Ribociclib, Abemaciclib.

In accordance with an embodiment, the present invention provides a method for identifying a female subject as having a breast tumor which is responsive to an anti-nucleolin agent comprising: a) testing a breast tumor tissue sample from the tumor of the subject for expression of CRYβB2 protein in the cells of the sample; b) comparing the level of expression in the sample with the level of expression in a reference breast tissue sample; and c) identifying the subject as having a breast tumor which is responsive to an anti-nucleolin agent when the level of expression of CRYβB2 protein in the cells of the sample is elevated compared to the level of expression of CRYβB2 protein in the cells of the reference breast tissue sample.

Nucleolin (NCL) is one of the most abundant nonribosomal proteins in the nucleolus, first identified in ribosomal RNA processing. Additional studies have demonstrated that NCL is a multifunctional nucleocytoplasmic protein, involved in ribosomal assembly, chromatin decondensation, transcription, nucleo-cytoplasmic import/export, and chromatin remodeling. NCL is frequently up-regulated in cancer and in cancer-associated endothelial cells compared with normal tissues, where it is also present on the cell surface. Altered NCL expression and localization results in oncogenic effects, such as stabilization of AKT, Bcl-2, Bcl-XL, and IL-2 mRNAs.

In accordance with another embodiment, the present invention provides a method for treating a female subject having a breast tumor which is responsive to an anti-nucleolin agent comprising: a) testing a breast tumor tissue sample from the tumor of the subject for expression of CRYβB2 protein in the cells of the sample; b) comparing the level of expression in the sample with the level of expression in a reference breast tissue sample; c) identifying the subject as having a breast tumor which is responsive to an anti-nucleolin agent when the level of expression of CRYβB2 protein in the cells of the sample is elevated compared to the level of expression of CRYβB2 protein in the cells of the reference breast tissue sample and d) administering to the subject an effective amount of an anti-nucleolin agent.

As used herein, the term “anti-nucleolin” means one or more agents which act to inhibit actions of NCL in the nucleolus. Examples of anti-nucleolin agents include, but are not limited to, anti-nucleolin antibodies, siRNAs, and nucleolin aptamer AS1411 aptamer and targeting peptides, including F3 peptide, NCL6 and HB-19 (23) (44).

In accordance with a further embodiment, the present invention provides a method for identifying a female as subject having a breast cancer tumor which is responsive to an inhibitor of ribosome RNA synthesis comprising: a) testing a breast cancer tissue sample from the tumor of the subject for expression of CRYβB2 protein in the cells of the sample; b) comparing the level of expression in the sample with the level of expression in a reference breast tissue sample; and c) identifying the subject as having a breast cancer tumor which is responsive to an inhibitor of ribosome RNA synthesis when the level of expression of CRYβB2 protein in the cells of the sample is elevated compared to the level of expression of CRYβB2 protein in the cells of the benign and/or reference breast tissue sample.

As used herein, the term “ribosomal RNA synthesis inhibitors” means one or more agents which are selective, and specific inhibitors of rRNA synthesis that suppresses Pol I transcription at the initiation stage and exhibits antiproliferative activity. Examples of such agents include, but are not limited to, CX-5461 (2-(4-methyl-[1,4]diazepan-1-yl)-5-oxo-5H-7-thia-1,11b-diaza-benzo[c]fluorene-6-carboxylic acid (5-methyl-pyrazin-2-ylmethyl)-amide) (45) and BMH-21 and its analogs (see WO2015/143293, incorporated by reference herein).

In accordance with another embodiment, the present invention provides a method for treating a female subject having a breast cancer tumor which is responsive to an inhibitor of ribosome RNA synthesis comprising: a) testing a breast cancer tissue sample from the tumor of the subject for expression of CRYβB2 protein in the cells of the sample; b) comparing the level of expression in the sample with the level of expression in a reference breast tissue sample; c) identifying the subject as having a breast cancer tumor which is responsive to an inhibitor of ribosome RNA synthesis when the level of expression of CRYβB2 protein in the cells of the sample is elevated compared to the level of expression of CRYβB2 protein in the cells of the benign and/or reference breast tissue sample and d) administering to the subject an effective amount of an inhibitor of ribosome RNA synthesis.

As used herein, the term “control sample” or “reference sample” means a sample from a subject known to have a CRYβB2 negative breast cancer, such as samples which were classified by a Pathologist as CRYβB2 negative by IHC.

The term “comparing” as used herein encompasses comparing the level of the peptide or polypeptide comprised by the sample to be analyzed with a level of a suitable reference level specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample or a ratio of amounts is compared to a reference ratio of amounts. The comparison referred to in the methods of the present invention may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format.

With respect to pharmaceutical compositions described herein, the pharmaceutically acceptable carrier can be any of those conventionally used, and is limited only by physico-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. Examples of the pharmaceutically acceptable carriers include soluble carriers such as known buffers which can be physiologically acceptable (e.g., phosphate buffer) as well as solid compositions such as solid-state carriers or latex beads. It is preferred that the pharmaceutically acceptable carrier be one which is chemically inert to the active agent(s), and one which has little or no detrimental side effects or toxicity under the conditions of use.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

In addition, in an embodiment, the compounds of the present invention may further comprise, for example, binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzalkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular compound, as well as by the particular method used to administer the compound. Accordingly, there are a variety of suitable formulations of the pharmaceutical composition of the invention. The following formulations for parenteral, subcutaneous, intravenous, intramuscular, intraarterial, intrathecal and intraperitoneal administration are exemplary, and are in no way limiting. More than one route can be used to administer the compounds, and in certain instances, a particular route can provide a more immediate and more effective response than another route.

Suitable soaps for use in parenteral formulations include, for example, fatty alkali metal, ammonium, and triethanolamine salts, and suitable detergents include, for example, (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the compounds in solution. Preservatives and buffers may be used. In order to minimize or eliminate irritation at the site of injection, such compositions may contain one or more nonionic surfactants, for example, having a hydrophile-lipophile balance (HLB) of from about 12 to about 17. The quantity of surfactant in such formulations will typically range from about 5% to about 15% by weight. Suitable surfactants include, for example, polyethylene glycol sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

For purposes of the invention, the amount or dose of the agents, salts, solvates, or stereoisomers, as set forth above, administered should be sufficient to effect, e.g., a therapeutic or prophylactic response, in the subject over a reasonable time frame. The dose will be determined by the efficacy of the particular compound and the condition of a human, as well as the body weight of a human to be treated.

The dose of the compounds, salts, solvates, or stereoisomers of any one the agents used in the inventive methods, as set forth above, of the present invention also will be determined by the existence, nature and extent of any adverse side effects that might accompany the administration of a particular agent. Typically, an attending physician will decide the dosage of the agent or agents with which to treat each individual patient, taking into consideration a variety of factors, such as age, body weight, general health, diet, sex, compound to be administered, route of administration, and the severity of the condition being treated. By way of example, and not intending to limit the invention, the dose of the one or more agents can be about 0.001 to about 1000 mg/kg body weight of the subject being treated/day, from about 0.01 to about 100 mg/kg body weight/day, or from about 1 mg to about 100 mg/kg body weight/day. In some embodiments the dosage of the one or more agents can be in the range of about 50 to 200 mg Palbociclib per day, or about 200 to 1000 mg Ribociclib per day, or about 50 to 300 mg Abemaciclib twice a day. In some embodiments the CDK4 inhibitors can be given for 1 to 4 weeks and then a week off, preferably the CDK4 inhibitors can be given for 3 weeks and then a week off.

Alternatively, the agents used in the methods of the present invention can be modified into a depot form, such that the manner in which the compound is released into the body to which it is administered is controlled with respect to time and location within the body (see, for example, U.S. Pat. No. 4,450,150). Depot forms of agents can be, for example, an implantable composition comprising the compound and a porous or non-porous material, such as a polymer, wherein the compound is encapsulated by or diffused throughout the material and/or degradation of the non-porous material. The depot is then implanted into the desired location within the body and the compounds are released from the implant at a predetermined rate.

It will be understood that the agents and methods described above in the inventive methods can be combined with one or more additional biologically active agents either serially or in combination.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

Further examples of biologically active agents include, without limitation, enzymes, receptor antagonists or agonists, hormones, growth factors, autogenous bone marrow, antibiotics, antimicrobial agents, RNA and DNA molecules and nucleic acids, and antibodies. Specific examples of useful biologically active agents the above categories include: anti-neoplastics such as androgen inhibitors, antimetabolites, cytotoxic agents, and immunomodulators.

Biologically active agents also include anti-cancer agents such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics.

Further examples of alkylating antineoplastic agents include carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

It will be understood by those of ordinary skill in the art that the methods of the present invention can be used to diagnose, prognosticate, and monitor treatment of any disease or biological state in which methylation of genes is correlative of such a disease or biological state in a subject. In some embodiments, the disease state is breast cancer. In some embodiments the type of breast cancer can be invasive ductal carcinoma or ductal carcinoma in situ.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream. In preferred embodiments, the cancers include breast, colon, prostate and lung cancer.

The cancer can be a metastatic cancer or a non-metastatic (e.g., localized) cancer, an invasive cancer or an in situ cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein.

Appropriate care in terms of breast cancer can constitute standard of care for treatment of breast cancer including, for example, surgery, surgery with post-operative radiation therapy, post-operative systemic therapy or chemotherapy depending on whether he tumor is hormone receptor negative or positive, the tumor is HER2/neu negative or positive, the tumor is hormone receptor negative and HER2/neu negative (triple negative), and the size of the tumor. Chemotherapy for breast cancer can include In premenopausal women with hormone receptor positive tumors, no more treatment may be needed or postoperative therapy may include: tamoxifen therapy with or without chemotherapy; tamoxifen therapy and treatment to stop or lessen how much estrogen is made by the ovaries; drug therapy, surgery to remove the ovaries, or radiation therapy to the ovaries may be used; aromatase inhibitor therapy and treatment to stop or lessen how much estrogen is made by the ovaries; and drug therapy, surgery to remove the ovaries, or radiation therapy to the ovaries may be used.

In postmenopausal women with hormone receptor positive tumors, no more treatment may be needed or postoperative therapy may include: aromatase inhibitor therapy with or without chemotherapy; tamoxifen followed by aromatase inhibitor therapy, with or without chemotherapy.

In women with hormone receptor negative tumors, no more treatment may be needed or postoperative therapy may include: chemotherapy.

In women with small, HER2/neu positive tumors, and no cancer in the lymph nodes, no more treatment may be needed. If there is cancer in the lymph nodes, or the tumor is large, postoperative therapy may include: chemotherapy and targeted therapy (trastuzumab); hormone therapy, such as tamoxifen or aromatase inhibitor therapy, for tumors that are also hormone receptor positive.

Drugs useful in the treatment of breast cancer include, but are not limited to: Abemaciclib; Abraxane (Paclitaxel Albumin-stabilized Nanoparticle Formulation); Ado-Trastuzumab Emtansine; Afinitor (Everolimus); Anastrozole; Aredia (Pamidronate Disodium); Arimidex (Anastrozole); Aromasin (Exemestane); Capecitabine; Cyclophosphamide; Docetaxel; Doxorubicin Hydrochloride; Ellence (Epirubicin Hydrochloride); Epirubicin Hydrochloride; Eribulin Mesylate; Everolimus; Exemestane; 5-FU (Fluorouracil Injection); Fareston (Toremifene); Faslodex (Fulvestrant); Femara (Letrozole); Fluorouracil Injection; Fulvestrant; Gemcitabine Hydrochloride; Gemzar (Gemcitabine Hydrochloride); Goserelin Acetate; Halaven (Eribulin Mesylate); Herceptin Hylecta (Trastuzumab and Hyaluronidase-oysk); Herceptin (Trastuzumab); Ibrance (Palbociclib); Ixabepilone; Ixempra (Ixabepilone); Kadcyla (Ado-Trastuzumab Emtansine); Kisqali (Ribociclib); Lapatinib Ditosylate; Letrozole; Lynparza (Olaparib); Megestrol Acetate; Methotrexate; Neratinib Maleate; Nerlynx (Neratinib Maleate); Olaparib; Paclitaxel; Paclitaxel Albumin-stabilized Nanoparticle Formulation; Palbociclib; Pamidronate Disodium; Perjeta (Pertuzumab); Pertuzumab; Ribociclib; Talazoparib Tosylate; Talzenna (Talazoparib Tosylate); Tamoxifen Citrate; Taxol (Paclitaxel); Taxotere (Docetaxel); Thiotepa; Toremifene; Trastuzumab; Trastuzumab and Hyaluronidase-oysk; Trexall (Methotrexate); Tykerb (Lapatinib Ditosylate); Verzenio (Abemaciclib); Vinblastine Sulfate; Xeloda (Capecitabine); Zoladex (Goserelin Acetate); and combinations thereof.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of diagnosis, staging, screening, or other patient management, including treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

Patient Samples, Cell Lines and Reagents.

Freshly resected breast tissue of women undergoing reduction mammoplasty and primary tumors from women undergoing treatment were provided by the Johns Hopkins Surgical Pathology Department, under protocols approved by the institutional review board. CD24⁺ and CD44⁺ cells were isolated from fresh normal breast tissue using magnetic beads as described previously (23). RNA and protein were generated from normal and breast cancer tissue (24). Breast cancer cells were obtained from the American Type Culture Collection. MCF10A-p53 null and knockin for p53-R248W and BRCA1-185delAG mutations were obtained from Ben H. Park. MCF10AT1 and DCIS.COM were obtained from Barbara Ann Karmanos Cancer Institute (Detroit, Mich.). Cells were authenticated using short tandem repeat (STR) profiling and tested for mycoplasma using MycoAlert PLUS mycoplasma detection kit (#LT07-218, Lonza). Palbociclib and homoharringtonine were purchased from Selleck Chemicals and Sigma Aldrich, respectively.

Constructs.

CRYβB2 coding sequence was cloned into a lentivirus vector (#17291, Addgene) using the Gateway Technology System (Thermo Fisher). MCF10A, MCF10AT1 and DCIS.COM cells overexpressing luciferase and CRYβB2 were generated following lentivirus infection. For immunofluorescence, MCF10AT1 cells were infected with lentivirus containing the CRYβB2 sequence tagged with the myc-DDK (flag) sequence (#RC210125, Origene). For CRISPR knockout nucleolin guide RNAs were designed using sgRNA online web page from Broad Institute and cloned into Lenticrispr V2 (#52961, Addgene). 293T cells were transfected with the lentivirus constructs using Lipofectamine (24) and virus were used to infect cancer cells.

Xenograft and Limiting Dilution Assay.

All animal studies were performed according to the guidelines and approval of the Animal Care Committee of the Johns Hopkins School of Medicine. Xenografts of DCIS.COM and MCF10AT1 cells expressing vector control and CRYβB2 and nucleolin knockout constructs were established in 6-8 weeks NOD-scid IL2Rgnull (NSG) mice (from an in-house colony at Hopkins) by injecting 5×10⁶ tumor cells into the fourth mammary gland. The mice bearing MCF10AT1 tumors were treated 1) for 2 weeks, receiving palbociclib (50 mg/kg) or saline, pH 4.0, as vehicle for 5 days/week orally or 2) for 5 weeks receiving homoharringtonine (HHT, 1 mg/kg) or saline as vehicle for 5 days/weeks i.p. The mice bearing MCF10AT1 tumors overexpressing CRYβB2 or TNBC HCC1806 cells CRYβB2-knockdown and control plasmids were daily intraperitoneal (i.p.) injected for 20 consecutive days or 7 weeks, respectively, with 150 μL of a sterile PBS solution containing 22 μg of AS-1411 or CRO. For limiting dilution assays, the tumors were digested with collagenase/hyaluronidase and single cells were injected at limiting dilutions (5×10⁶-1×10⁵) into mammary fat pads (24). The CSC frequency was estimated using Extreme Limiting Dilution Analysis (ELDA) (25). Bioluminescence imaging was performed using IVIS system (26).

Western Blot, Immunohistochemistry, Immunofluorescence and Senescence Assay.

Western blotting, immunohistochemistry (IHC) (24) and immunofluorescence (IF) (27) were performed as previously described using antibodies against CRYβB2 (Western cell lines and patient samples: #SC-376006, Santa Cruz Biotechnology and #PA5-60496, Thermo Fisher, respectively); IHC: #NBP2-13876, Novus Biologicals), EMT markers, cell cycle, apoptosis and DNA damage (detailed in Supplemental Methods) (24). For IF, the slides were probed with the following primary antibodies: CRYβB2 (#PA5-60496, Thermo Fisher), CRYβB2-flag (#14793, Cell Signaling Technology), organelle-specific antibodies (detailed in Supplemental Methods) and nuclear staining (Hoechst 33342; Fisher Scientific) (27). ImageJ was used for quantification. Sections of MCF10AT1 tumors expressing CRYβB2 and control plasmids were stained with senescence beta-galactosidase staining Kit (Cell Signaling, #9860S).

Immunolabeling for CRYβB2 was performed by the Oncology Tissue Services Core of Johns Hopkins University on formalin-fixed, paraffin embedded sections. Briefly, following dewaxing and rehydration, slides were immersed in 1% Tween-20, then heat-induced antigen retrieval was performed in a steamer using Target Retrieval Solution (sodium citrate buffer pH 6.0, catalog #S170084-2, Dako) for 45 minutes. Slides were rinsed in PBST and endogenous peroxidase and phosphatase was blocked (catalog #S2003, Dako) and sections were then incubated with primary antibody rabbit anti-CRYβB2 (#NBP2-13876, Novus Biologicals, 1:100) for overnight at 4 degrees. The primary antibodies were detected by 30 minute incubation with HRP-labeled anti-rabbit secondary antibody (catalog #PV6119, Leica Microsystems) followed by detection with 3,3′-Diaminobenzidine (catalog #D4293, Sigma-Aldrich), counterstaining with Mayer's hematoxylin, dehydration and mounting.

Bioinformatics Analysis.

A total of 1222 bam files corresponding to RNA seq results for TCGA breast tumors were remotely sliced to include reads for CRYβB2 and CRYβB2P1, and downloaded from the Genomic Data Commons (GDC) data portal (portal.gdc.cancer.gov/). Matching HT-seq count files, clinical data, and pam50 subtypes were downloaded from the GDC as well. Total read counts for each sample were obtained by summing over all genes in the HT-seq count files. Standardized reads per million were separated into 3 groups: those with alignments only in CRYβB2, those with alignments only in CRYβB2P1 and those with alignments in both. Race specific differences in expression were evaluated by Wilcoxon rank sum test and visualized using boxplots.

Transcriptome Array.

RNA from MCF10AT1 and DCIS.COM cells overexpressing CRYβB2 or vector control was extracted using RNeasy Mini Kit (Qiagen) and Agilent Human Genome CGH Microarray 4×44K was performed by the Microarray Core at Johns Hopkins. Microarray data was preprocessed by background subtraction followed by quantile normalization using GenomeStudio. After pre-processing using GenomeStudio, data was imported and analyzed using R (using base and Bioconductor packages) and Pathway Analysis was performed using gene set variant analysis (GSVA) (28) on Hallmark gene sets defined by Molecular Signatures Database (MSigDB).

Human Proteome Microarray.

MCF10AT1-overexpressing CRYβB2 cell lysate was briefly sonicated in cell lysis buffer (10 mM Tris, 150 mM NaCl, 2 mM CaCl₂, and 2 mM MgCl₂, protease inhibitor mixture, pH=7.5). The cell debris was removed by centrifugation at 13,000 rpm for 10 min at 4° C. The protein amount in the supernatant was quantified by the absorbance at 600 nm to estimate the protein concentration with a BCA reagent. HuProt™ Human Proteome Microarray v3.0 (CDI lab, US) containing 20,240 individually purified full-length human proteins was blocked by the binding buffer (10 mM Tris, 150 mM NaCl, 2 mM CaCl₂, and 2 mM MgCl₂, pH=7.5) with 5% BSA at room temperature for 1 hour. The cell lysate was diluted to a final concentration of 10 mg/mL in 3 mL 5% BSA binding buffer and incubated on the microarray for 2 hours. After three times 5-min washes, the chip was incubated with CRYβB2 antibody (#sc-376006, Santa Cruz Biotechnology) and secondary antibody. A microarray scanner (GenePix 4000B) was applied to scan the microarray, and the CRYβB2 binding signals were acquired and analyzed using Genepix 7.0. In the data analysis, the median values of Signal (Sᵢⱼ) and background intensities (Bᵢⱼ) at site of each spots (i,j) on the scanning result were extracted, respectively. The binding intensity of each protein spot was defined as the ratio of Sᵢⱼ and Bᵢⱼ (SNBᵢⱼ) and the hits identification as previously described (29,30) (29).

Bioluminescence Imaging.

For imaging in vivo, each mouse received 150 mg of D-luciferin per kg of body weight. Mice were anesthetized using isoflurane gas (2% in oxygen at 0.6 l/min flow rate) throughout imaging, and images were collected at indicated times (normally 5 or 20 min) after D-luciferin injection (monitored up to 40 min after the injection). Composite images obtained were comprised of black and white digital photos with an overlay of images reflecting bioluminescent intensity. The density map, measured as photons/second/cm²/steradian (p/s/cm²/sr), were created using the Xenogen software and represented as a color gradient centered at the maximal spot. A sum in the ROI was also automatically selected and calculated by the software.

Co-Immunoprecipitation.

The Thermo Scientific Pierce Co-Immunoprecipitation Kit (#26149) was used for co-immunoprecipitation of CRYβB2 and its interaction partners. Antibody against CRYβB2 (#SC-376006, Santa Cruz Biotechnology) was covalently coupled onto an amine-reactive resin. CRYβB2 interaction with its partners in protein lysates from MCF10AT1 vector and CRYβB2—expressing cells were developed by SDS-PAGE analysis using antibodies against nucleolin (#14574, Cell Signaling), PAIP1 (ab175211, Abcam) and GRB2 (#3972, Cell Signaling).

Statistical Analysis.

The results of cell culture experiments were expressed as mean±standard errors of mean (SEM). Two-tailed Student's T-tests (95% confidence interval) were performed on pairwise combinations of data to determine statistical significance defined as *p<0.05, **p<0.01 and ***p<0.001. qRT-PCR using tumor xenografts results were expressed using the median and two-tailed Mann Whitney Test. Statistical analyses were performed using GraphPad Prism version 5.0 (GraphPad Software, Inc.).

Cell proliferation assay. Cells proliferation assay was performed as previously described (31). Cells were grown in 100 mm plates (500 cells/plate), fixed with formalin, and stained with 0.05% crystal violet. To quantitate growth, the dye was solubilized using acetic acid, and absorbance was measured at 590 nm.

Soft agar assay. Anchorage-independent growth was assessed by seeding 5,000 cells on soft agar (0.4% top layer, 0.8% bottom layer); and counting the colonies after 14 days using an inverted microscope and 0.005% crystal violet for staining.

Tumor Sphere. Tumor sphere assays were performed as previously described (32), with modifications. Briefly, 1×10⁴ cells were seeded in 24-well ultra-low adhesion plates (Corning) in 1 ml of mammary epithelial growth medium (MEGM, Lonza) containing supplements (24).

Surface sensing of translation. Surface sensing of translation (SUnSET) was performed as previously described (Schmidt et al., 2009) with minor modifications: cells were incubated with 10 μg/ml of puromycin in cell culture media for 1 h before harvest. Proteins were analyzed using immunoblots.

Xenograft and Limiting Dilution Assay. All animal studies were performed according to the guidelines and approval of the Animal Care Committee of the Johns Hopkins School of Medicine. Xenografts of DCIS.COM and MCF10AT1 cells expressing vector control and CRYβB2 and nucleolin knockout constructs were established in 6-8 weeks NOD-scid IL2Rgnull (NSG) mice (from an in-house colony at Hopkins) by injecting 5×10⁶ tumor cells into the fourth mammary gland. The mice bearing MCF10AT1 tumors were treated 1) for 2 weeks, receiving palbociclib (50 mg/kg) or saline, pH 4.0, as vehicle for 5 days/week orally, or 2) for 5 weeks receiving homoharringtonine (HHT, 1 mg/kg) or saline as vehicle for 5 days/weeks i.p. For limiting dilution assays, the tumors were digested with collagenase/hyaluronidase and single cells were injected at limiting dilutions (5×10⁶-1×10⁵) into mammary fat pads (55). The CSC frequency was estimated using Extreme Limiting Dilution Analysis (ELDA) (56). Bioluminescence imaging was performed using IVIS system (57).

### Example 1

CRYβB2 is upregulated in breast tumors of AA patients and is expressed in stem-like cells.

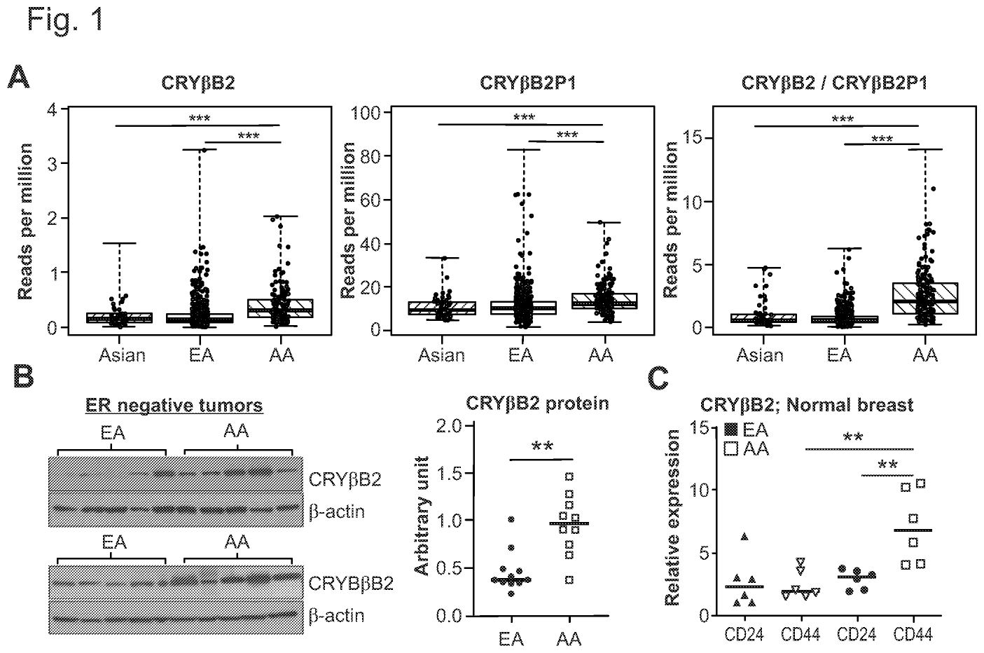

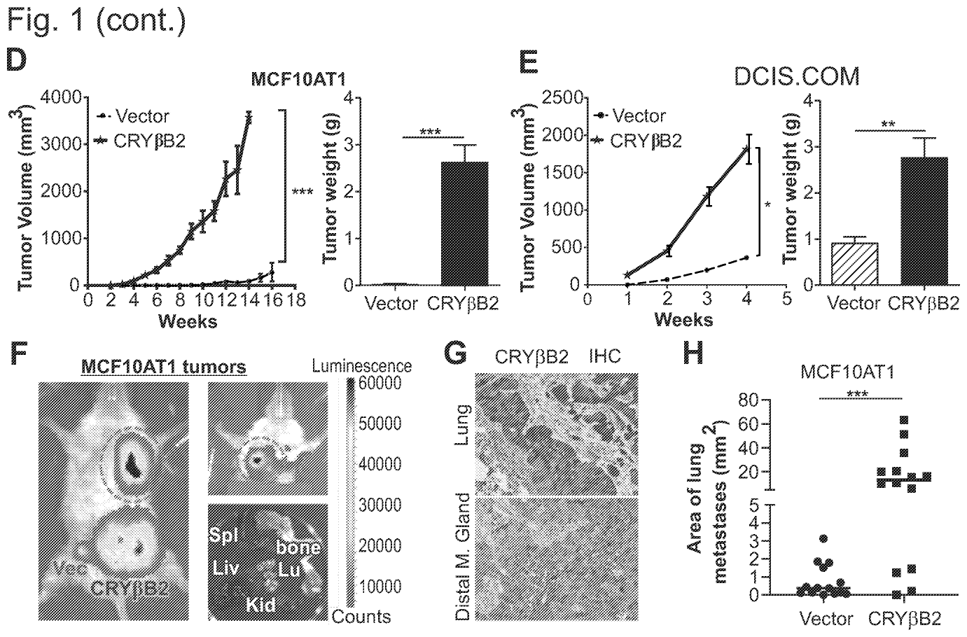

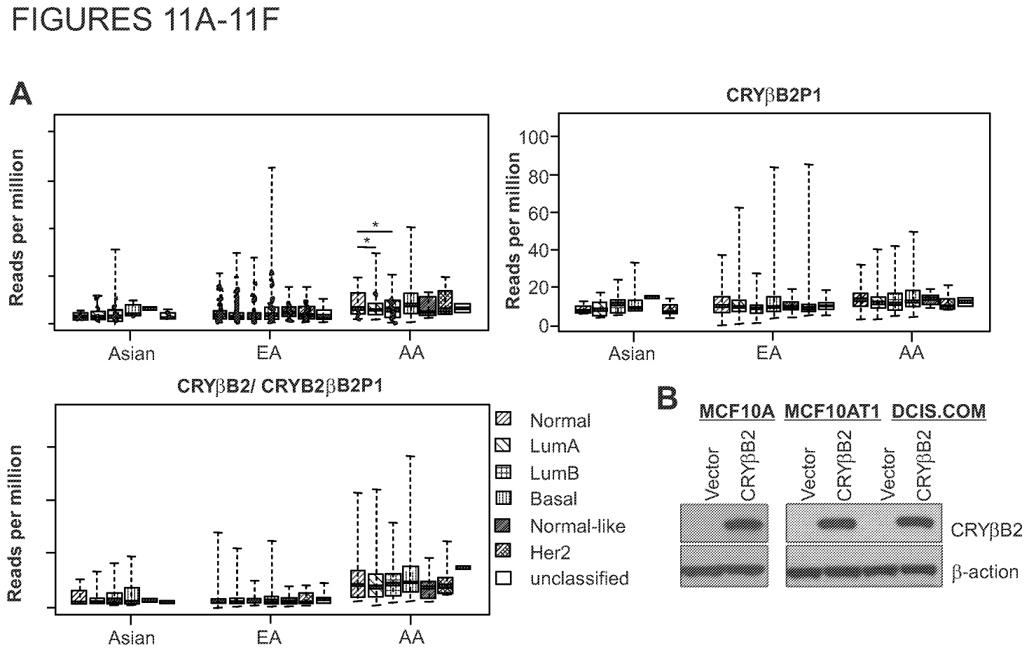

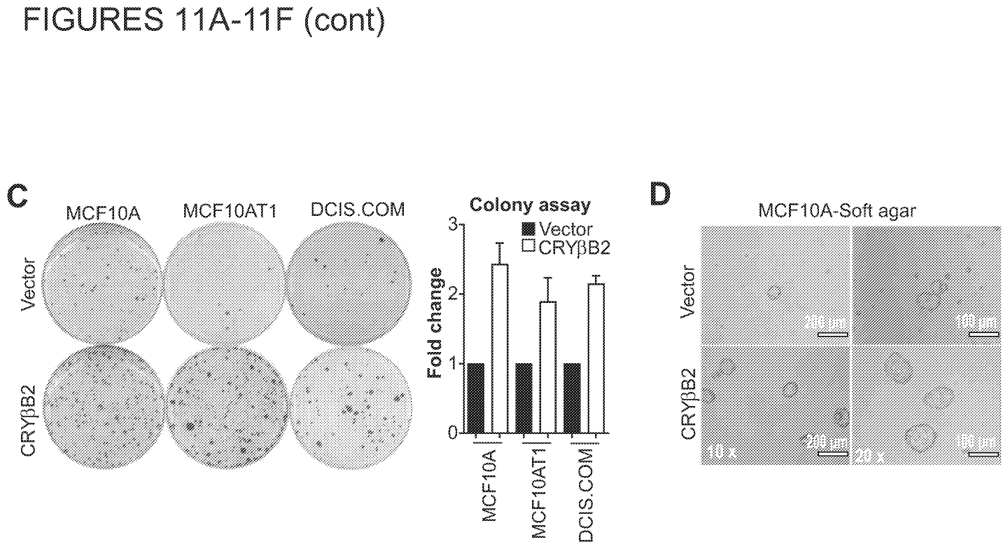

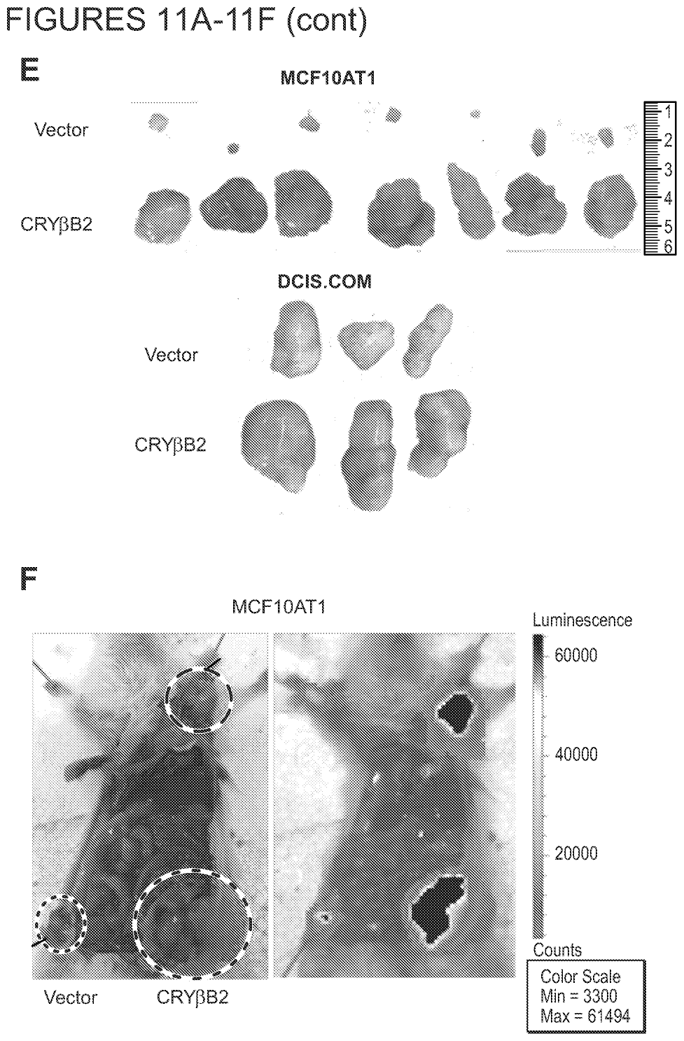

Similar to CRYβB2, its pseudogene, CRYβB2P1, was also shown to be induced in AA breast tumors (33). Due to their partial sequence similarity and technical limitations to distinguish both genes in expression arrays (15), there is a need to confirm whether both genes are indeed differentially expressed accordingly to race. Analyzing TCGA breast tumor RNA seq data using their own custom scripts, Barrow et al., observed that only CRYβB2P1 is differentially expressed in AA tumors (15). We repeated this analysis, using the BAM-slicing function available through the Genomic Data Commons Portal (portal.gdc.cancer.gov/), to download reads aligning to CRYβB2 and/or CRYβB2P1 (FIG. 1A). Reads were classified into 3 categories: those mapping uniquely to CRYβB2, those mapping uniquely to CRYβB2P1, and those mapping to both. We observed that the pseudogene, CRYβB2P1 was more highly expressed than CRYβB2, but that the expression of both CRYβB2 and CRYβB2P1 was significantly higher in tumors of AA women when compared with Asian and EA women (FIG. 1A and Table 1). Moreover, the basal-like breast cancer subtype tend to express higher levels of CRYβB2 and CRYβB2P1 in comparison to normal breast in all 3 race/ethnic groups (FIG. 11A). Most importantly, we observed that CRYβB2 protein expression is significantly higher in estrogen receptor (ER)-negative tumors in AA in comparison to EA (FIG. 1B). While our findings are generally consistent with Barrow et al., our data provide additional evidence that not only CRYβB2P1 but also CRYβB2 is up-regulated in AA tumors.

Next, since CRYβB2 is involved in regeneration of cells (19), we asked the question whether expression of CRYβB2 could be related to stemness. Accordingly, we observed that CRYβB2 is upregulated exclusively in progenitor/stem cells (CD44+) that were isolated from six normal breast tissues of AA women (FIG. 1C).

Expression of CRYβB2 and CRYβB2P1 mRNA in TCGA breast tumors of Asian, European American (EA) and African American (AA).

| | | | | Wilcoxon Rank | | Wilcoxon | Wilcoxon |
|---|---|---|---|---|---|---|---|
| | | | | Sum | Wilcoxon | Rank Sum | Rank Sum |
| | | | | Statistic | Rank Sum | Statistic | P-value |
| | | | | AA vs | P-value | AA vs | AA vs |
| Gene | Asian | AA | EA | EA | AA vs EA | Asian | Asian |
| CRYβB2 | 0.3 | 13.29 | 2.44 | 67801 | <0.000001 | 5523 | <0.000001 |
| CRYPβ2P1 | 0.15 | 8.75 | 0.55 | 63769 | <0.000001 | 5311 | 0.000001 |
| BOTH | 0.14 | 10.04 | 0.59 | 81319 | <0.000001 | 6182 | <0.000001 |

Columns 2-4: median normalized (per million) reads per sample; Columns 5-6; Wilcoxon rank sum test results for comparison of AA and EA subjects; Columns 7-8; Wilcoxon rank sum test results for comparison of AA and Asian subjects.

### Example 2

CRYβB2 promotes tumorigenesis of low malignant breast cells.

In order to identify the primary role of CRYβB2 in breast tumorigenesis, without the influence of additional oncogenes such as the ones driving proliferation in TNBC cells (17), we overexpressed this AA-associated gene in: 1) immortalized human mammary epithelial cells, MCF10A, and low malignant, MCF10A cells that contained: 2) a mutated HRAS gene, MCF10AT1(34), and 3) mutated HRAS and PIK3CA genes, DCIS.COM (35) (FIG. 11B). Using these three cell lines, we observed that CRYβB2—overexpressing cells formed, on average, two times more colonies on plates compared to vector-transfected cells (FIG. 11C). Thus, CRYβB2 overexpression increased cell proliferation of normal and low malignant cells.

Although CRYβB2 increased anchorage-independent proliferation of normal MCF10A cells (FIG. 11D), it was not sufficient to induce their transformation, evidenced by an inability of the cells to form tumors in immunodeficient mice. On the contrary, MCF10AT1 and DCIS.COM cells overexpressing CRYβB2 formed significantly larger tumors than vector control cells (FIG. 1D and FIG. 1E). As shown in FIG. 1F, FIG. 1G, and FIG. 11F MCF10AT1-CRYβB2 cells systemically invaded into distal mouse mammary glands and metastasized to the lung and bone. The lung lesions originated from CRYβB2 metastatic cells were significantly bigger than CRYβB2-negative lesions (FIG. 1H).

### Example 3

CRYβB2 increases nucleoli size, stromal recruitment and epithelial to mesenchymal transition in breast tumors.

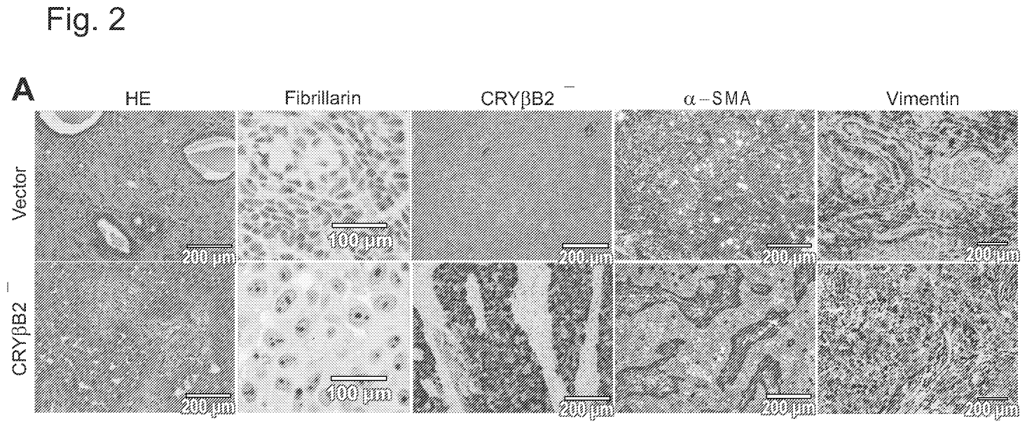

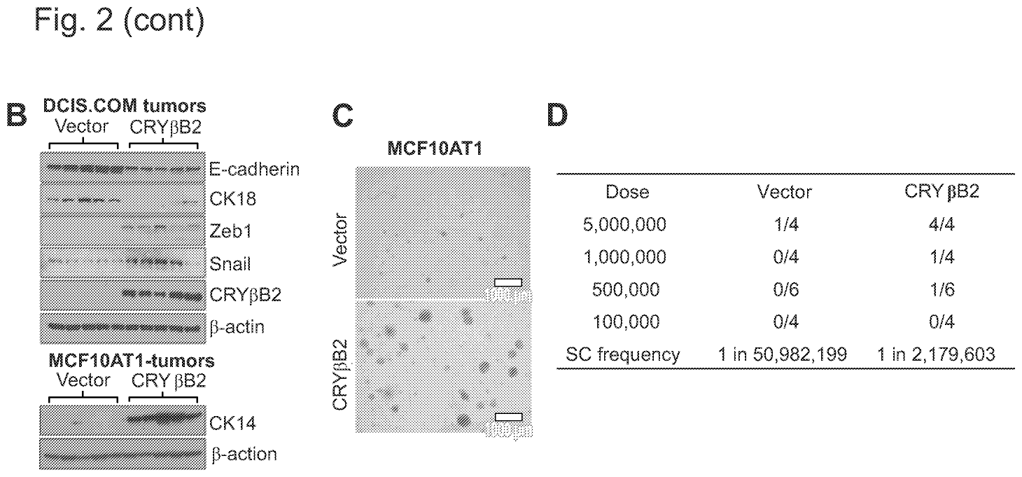

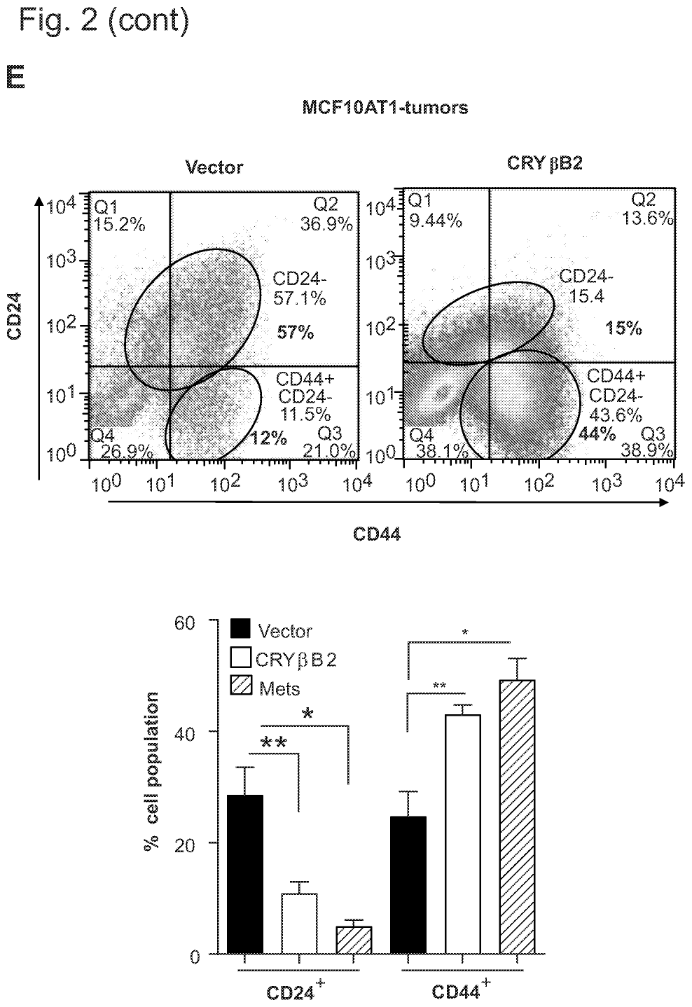

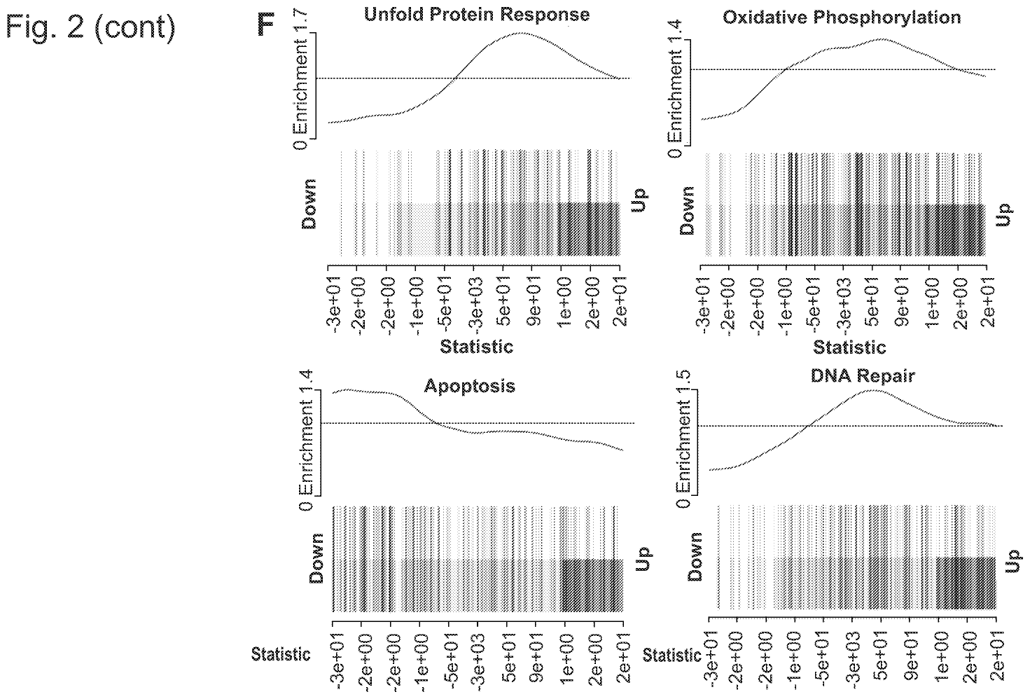

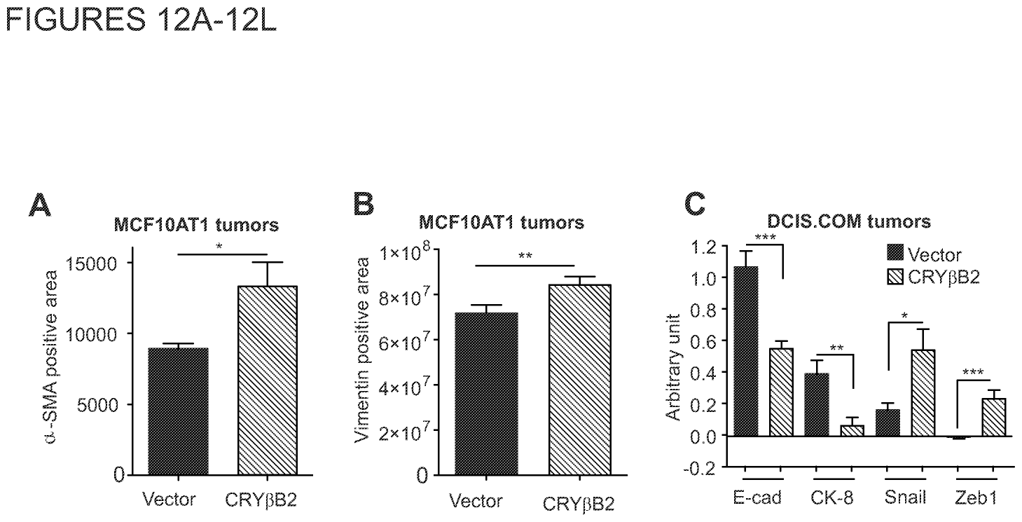

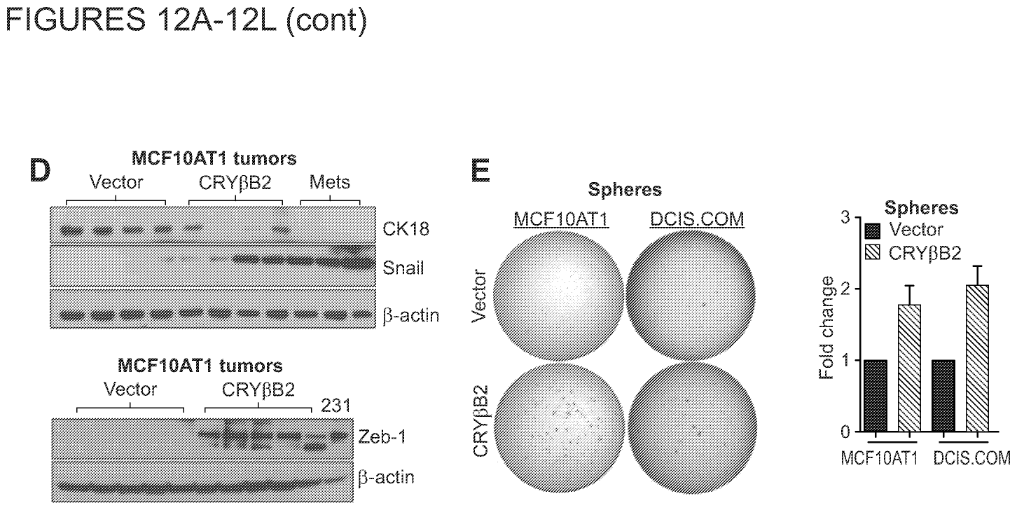

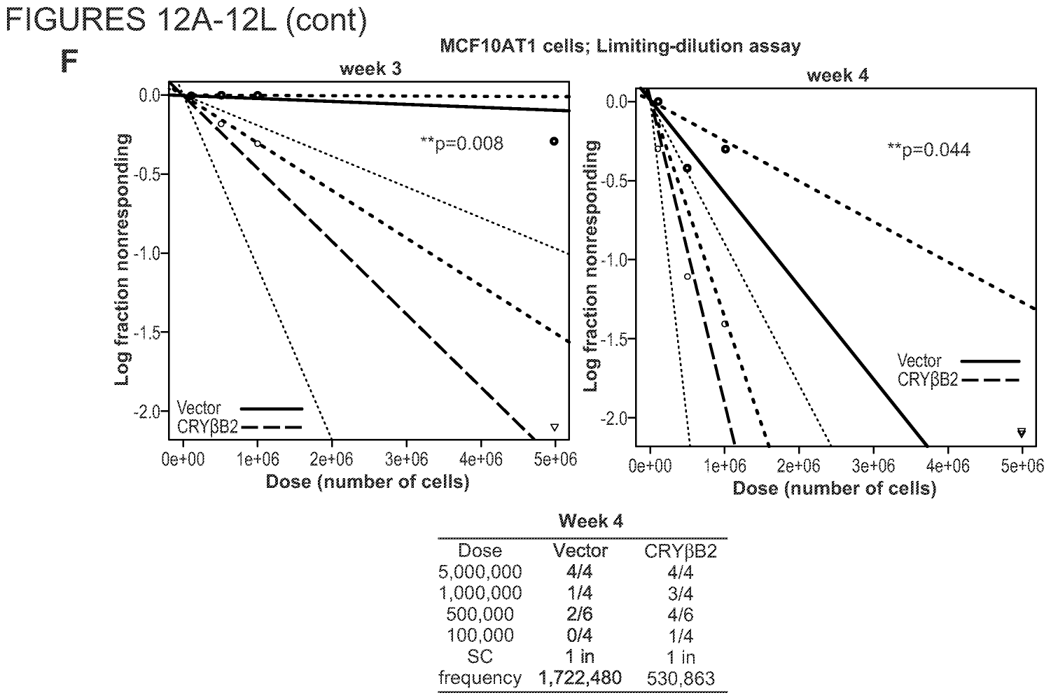

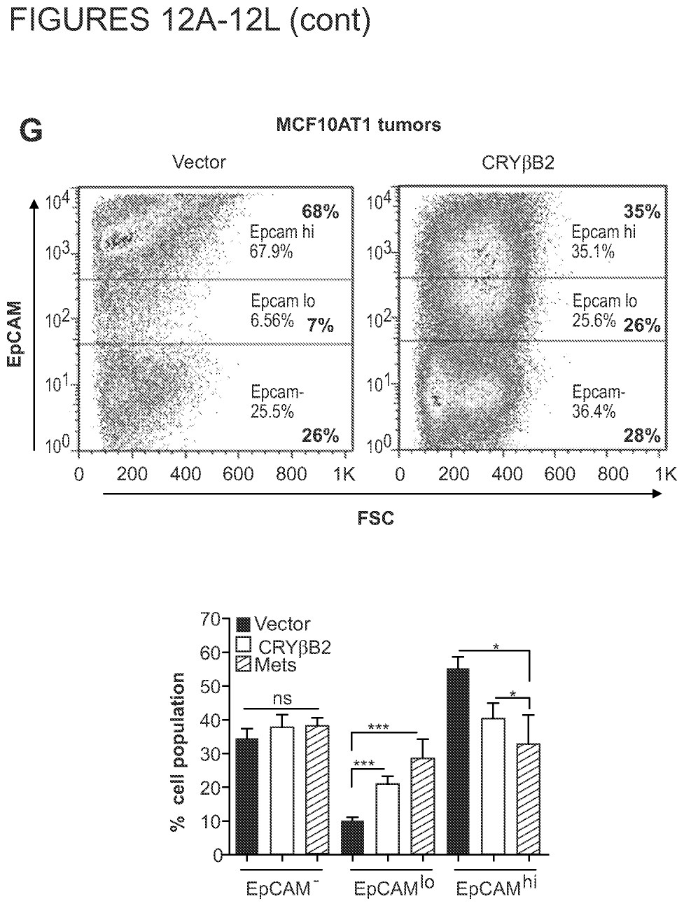

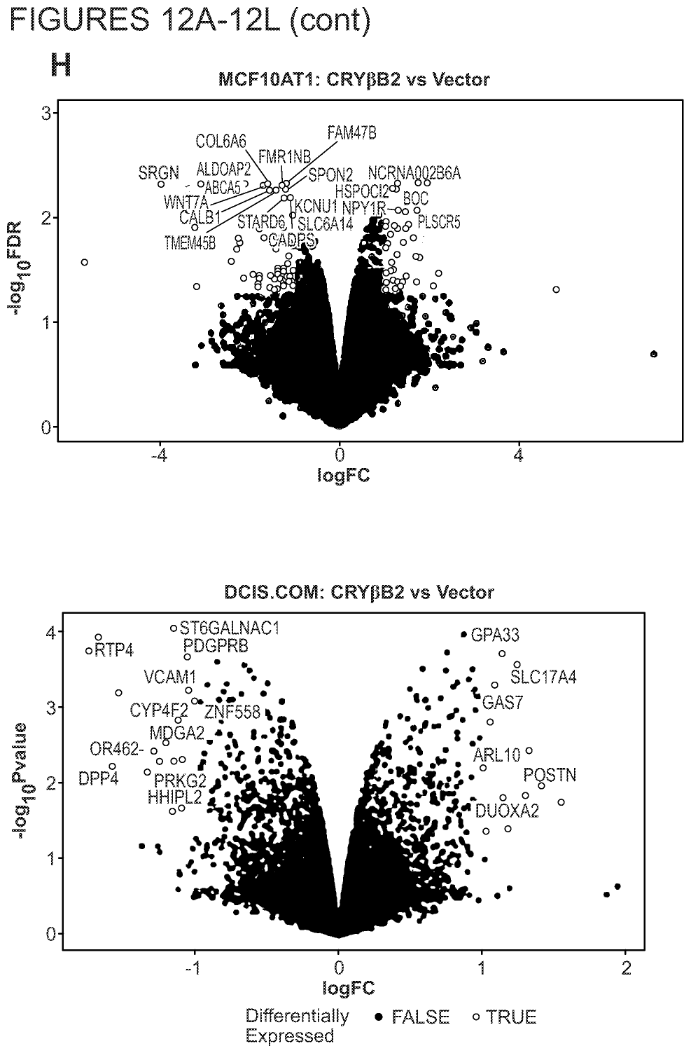

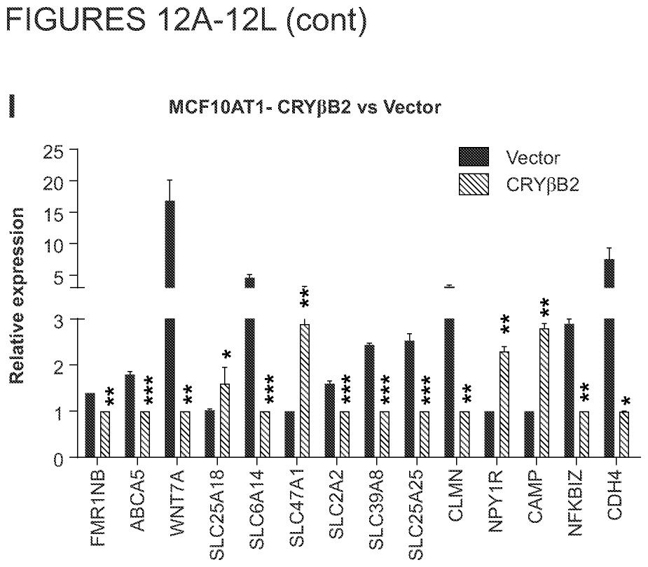

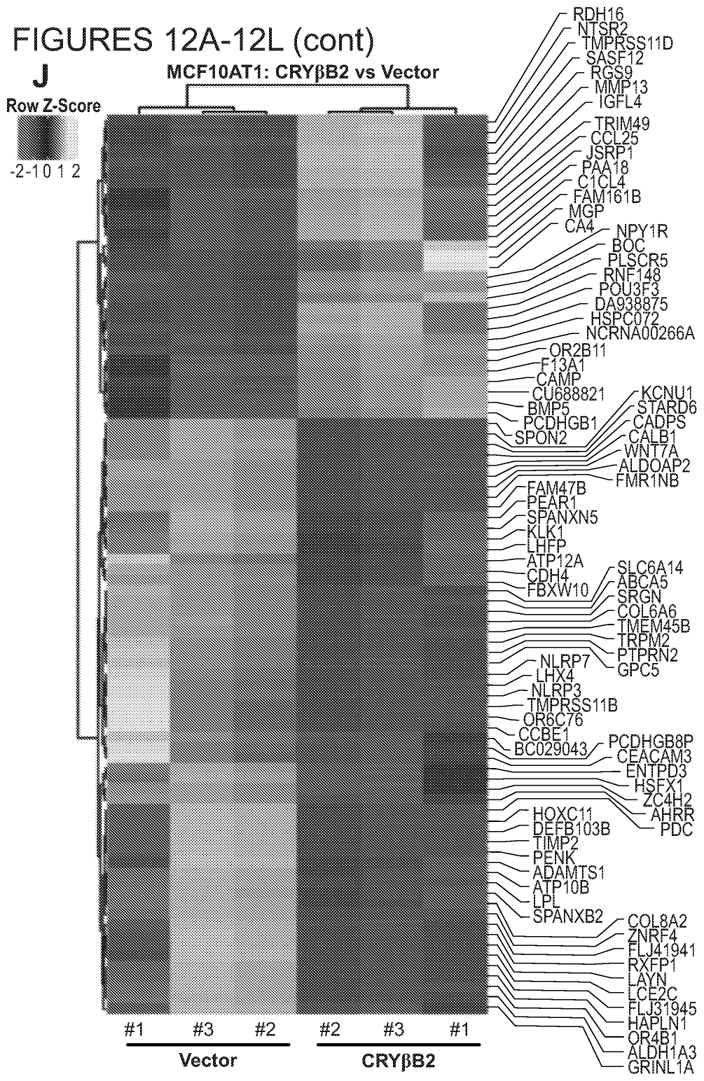

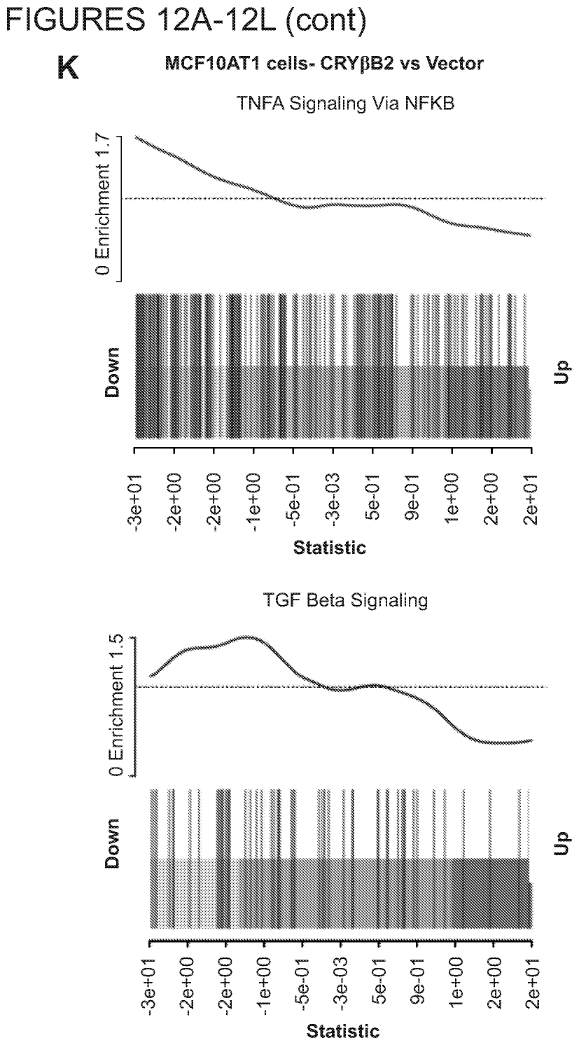

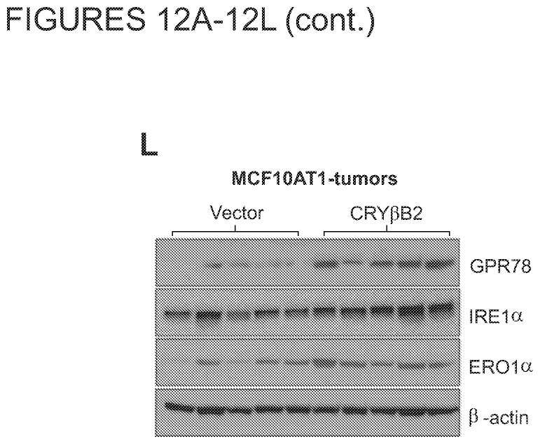

In order to decipher the mechanisms by which CRYβB2 mediates increased malignancy, we analyzed tumor morphology. Using histopathology, we observed that MCF10AT1-CRYβB2 tumors are less differentiated and resemble squamous cell-like carcinoma while MCF10AT1-vector tumors are more differentiated and predominantly express features of adenocarcinoma (FIG. 2A). MCF10AT1-CRYβB2 tumors had an increase in the number and size of nucleoli and the nuclei, in comparison to MCF10AT1-vector tumors, as revealed by an increase in fibrillarin staining (FIG. 2A). We also observed an increase in cancer-associated fibroblasts (CAF) which were CRYβB2—and alpha smooth muscle actin (α-SMA)⁻ in MCF10AT1-CRYβB2 tumors in comparison to vector (FIG. 2A and FIG. 12A). Expression of the mesenchymal marker vimentin increased in MCF10AT1-CRYβB2 tumors and stained both elongated mouse stromal cells and tumor cells with large nuclei (FIG. 2A and FIG. 12B).

Moreover, we observed downregulation of the epithelial markers E-cadherin and cytokeratin-18 and upregulation of the mesenchymal markers cytokeratin-14, Snail/Slug and Zeb1 in DCIS.COM (FIG. 2B and FIG. 12C) and MCF10AT1-CRYβB2 (FIG. 2B and FIG. 12D) tumors in comparison to vector controls. Together, these results showed that CRYβB2 induces features related to aggressive disease including an increase in nucleoli size, epithelial to mesenchymal transition (EMT) and stromal recruitment.

### Example 4

CRYβB2 Increases Cancer Stem Cell Number in Breast Tumors

Since we observed an increase in CRYβB2 expression in stem/progenitor cell population isolated from normal breast tissue from AA women (FIG. 1C), we sought to investigate its effect on self-renewal of human mammary cells. We observed that MCF10AT1 and DCIS.COM cells overexpressing CRYβB2 formed on average 2-times more tumor-spheres compared to vector controls (FIG. 2C and FIG. 12E). These results suggest that CRYβB2 could be involved in the expansion of cancer stem cells (CSC). Consistent with this hypothesis, MCF10AT1-CRYβB2 cells contained higher number of CSC and were significantly more efficient in engraftment into mammary fat pads of immunodeficient mice than vector cells (FIG. 2D and FIG. 12F).

To further buttress these findings, we investigated whether CRYβB2 tumors show an increase in CSC markers. We observed a significant decrease of differentiated cells (EpCAM⁺/CD24⁺) and an increase in stem/progenitor cells (CD44⁺/CD24⁻) in MCF10AT1-CRYβB2 tumors and mammary gland metastases (FIG. 2E and FIG. 12G). Together, these results showed that CRYβB2 increased the number of cells with CSC characteristics.

### Example 5

CRYβB2 regulates genes associated with an increase in malignant properties.

To explore additional pathways of tumor aggression initiated by CRYβB2, we performed a high-throughput gene expression profiling analysis of MCF10AT1 and DCIS.COM cells overexpressing CRYβB2 and vector controls.

Differential expression analysis identified robust gene expression changes in both MCF10AT1-CRYβB2 and DCIS.COM-CRYβB2 cells (FIG. 2F and FIG. 12H). CRYβB2 decreased expression of genes with tumor suppressor function, such as FMR1NB, ABCA5, Wnt7A, CLMN, NFKBIZ and CDH4 and increased expression of oncogenic genes, such as NPY1R and CAMP in MCF10AT1 cells (FIG. 12I and FIG. 12J).

A comprehensive analysis of the pathways regulated by CRYβB2 identified genes related to unfolded protein response, oxidative phosphorylation and DNA repair pathways and a decrease in genes related to apoptosis in the low malignant MCF10AT1 cells (FIG. 2F, FIG. 12K and Table 2). We also found that MCF10AT1-CRYβB2 tumors have increased levels of proteins that are activated during endoplasmic reticulum stress response as a consequence of expression of unfolded proteins (36), such as G protein-coupled receptor 78 (GPR78), inositol-requiring enzyme-1a (IRE1a) and endoplasmic reticulum oxidoreductase 1 alpha (ERO1a) (FIG. 12L). These results suggest that CRYβB2 may regulate the unfolded protein response.

TABLE 2
Gene expression array and pathways analysis of MCF10AT1-
CRYβB2 cells in comparison to vector cells.

| Pathways | pvals..5 | pvals.adj..5 | N | genes | Direction |
|---|---|---|---|---|---|
| UNFOLDED_PROTEIN_RESPONSE | 0.00001 | 0.00029 | 113 | HSP90B1, HYOU1, | up |
| | | | | DCTN1, SSR1 | |
| OXIDATIVE_PHOSPHORYLATION | 0.00089 | 0.022 | 199 | UQCRC1, | up |
| | | | | NDUFS7, | |
| | | | | NDUFV1, ACO2, | |
| | | | | FH, NDUFA5, | |
| | | | | MRPL11, | |
| | | | | HSD17B10 | |
| DNA_REPAIR | 0.0017 | 0.028 | 149 | GTF2F1, VPS37D, | up |
| | | | | SEC61A1, PDE6G, | |
| | | | | REV3L | |
| TNFA_SIGNALING_VIA_NFKB | 0 | 0.00004 | 200 | SGK1, DENND5A, | down |
| | | | | ATP2B1, TNC | |
| TGF_BETA_SIGNALING | 0.0043 | 0.027 | 54 | SMAD7, TGFB1 | down |
| KRAS_SIGNALING_UP | 0.0002 | 0.0025 | 200 | SPP1, IGF2, | down |
| | | | | RGS16, EPHB2, | |
| | | | | F13A1 | |
| INFLAMMATORY_RESPONSE | 0.00245 | 0.017 | 200 | CCL7, IL15, OSM, | down |
| | | | | ATP2B1, RGS16, | |
| | | | | MSR1, P2RX4 | |
| ESTROGEN_RESPONSE_LATE | 0.00213 | 0.017 | 200 | NPY1R, PDLIM3, | down |
| | | | | SOX3, SLC26A2, | |
| | | | | HSPB8, SGK1, | |
| | | | | SLC2A8, | |
| | | | | SERPINA1 | |
| ESTROGEN_RESPONSE_EARLY | 0.00002 | 0.00037 | 199 | NPY1R, HSPB8, | down |
| | | | | SLC26A2, | |
| | | | | TTC39A, ASB13, | |
| | | | | CELSR1, PDLIM3, | |
| | | | | SYT12, SOX3, | |
| | | | | NAV2 | |
| COMPLEMENT | 0.00572 | 0.032 | 199 | MMP13, C1QA, | down |
| | | | | KLKB1, | |
| | | | | SERPINA1, GP9, | |
| | | | | FCN1, PHEX, | |
| | | | | NOTCH4 | |

Gene Set Enrichment Analysis (GSEA) of differentially expressed genes and regulated pathways in MCF10AT1-CRYβB2 cells in comparison to vector. The different pathways, p value, adjusted p value, number of genes differentially expressed, strongly regulated genes and direction of pathway regulation are shown.

Overexpression of CRYβB2 in the more malignant DCIS.COM cells induced pathways related to apoptosis (TNF-α), Wnt/β-catenin and EMT and downregulated cell cycle (TP53, G2M checkpoint) pathways (Table 3). This data is in accordance with the induction of EMT and sternness in CRYβB2 tumors (FIG. 2) and the previously described correlation of CRYβB2 with Wnt pathway signaling (37).

TABLE 3
Gene expression array and pathways analysis of DCIS.COM-CRYβB2 cells in comparison to vector cells.

| Pathways | pvals | pvals.adj | N | genes | direction |
|---|---|---|---|---|---|
| TNFA_SIGNALING_VIA_NFKB | 0 | 3.00E−05 | 200 | JUNB, ATF3, | up |
| | | | | PTGS2, CD83, | |
| | | | | PLAUR, JUN, | |
| | | | | EGR3, RELB, | |
| | | | | EGR1, FOSL1, | |
| | | | | NR4A1, GCH1, | |
| | | | | DUSP2, | |
| | | | | GADD45A, | |
| | | | | BHLHE40, EGR2, | |
| | | | | TRIB1, VEGFA, | |
| | | | | AREG, KLF4, | |
| | | | | RNF19B, PDE4B, | |
| | | | | CD80, PER1, | |
| | | | | BMP2, GFPT2 | |
| APICAL_JUNCTION | 1.00E−05 | 0.00016 | 200 | PVRL3, ITGA3, | up |
| | | | | SORBS3, ACTN2, | |
| | | | | EPB41L2, | |
| | | | | ADRA1B, CDSN, | |
| | | | | ITGA10, SLIT2, | |
| | | | | CRAT, CDH15, | |
| | | | | SLC30A3, ICAM5, | |
| | | | | KCNH2, NRTN, | |
| | | | | AMIGO1 | |
| KRAS_SIGNALING_UP | 0.00107 | 0.01785 | 200 | SPRY2, DUSP6, | up |
| | | | | TFPI, ADAM8, | |
| | | | | MMP10, IGFBP3, | |
| | | | | ETV1, CLEC4A, | |
| | | | | ITGBL1, BMP2, | |
| | | | | C3AR1, PLAUR, | |
| | | | | AKAP12, SCG3, | |
| | | | | SEMA3B, BTC, | |
| | | | | KLF4, TRIB1, | |
| | | | | LAT2, PTGS2, | |
| | | | | LCP1, EREG, | |
| | | | | ANKH, ELTD1, | |
| | | | | PLVAP, GFPT2, | |
| | | | | CXCR4 | |
| PANCREAS_BETA_CELLS | 0.00155 | 0.01937 | 40 | NKX6-1, DCX, | up |
| | | | | FOXA2, PDX1, | |
| | | | | PAK3 | |
| EPITHELIAL_MESENCHYMAL_TRANSITION | 0.00317 | 0.03102 | 200 | FN1, IGFBP3, | up |
| | | | | LOX, NNMT, | |
| | | | | BGN, SERPINH1, | |
| | | | | POSTN, EDIL3, | |
| | | | | EMP3, VEGFA, | |
| | | | | GADD45A, | |
| | | | | SERPINE2, | |
| | | | | SGCD, | |
| | | | | TNFRSF12A, | |
| | | | | PLAUR, SFRP1, | |
| | | | | FBLN2, JUN, | |
| | | | | FERMT2, | |
| | | | | PDLIM4, ECM2, | |
| | | | | AREG, SLIT2, | |
| | | | | WIPF1 | |
| WNT_BETA_CATENIN_SIGNALING | 0.00372 | 0.03102 | 42 | AXIN2, NOTCH4, | up |
| | | | | WNT6 | |
| OXIDATIVE_PHOSPHORYLATION | 0 | 0 | 199 | VDAC3, | down |
| | | | | TIMM17A, | |
| | | | | ATP6V1E1, IDH1, | |
| | | | | PRDX3 | |
| MYC_TARGETS_V1 | 0 | 0 | 200 | PRDX3, VDAC3 | down |
| G2M_CHECKPOINT | 0 | 2.00E−05 | 199 | CENPF, CCNF, | down |
| | | | | HMGB3, PBK, | |
| | | | | EGF, HSPA8, | |
| | | | | PTTG3P | |
| E2F_TARGETS | 1.00E−05 | 7.00E−05 | 198 | DLGAP5, | down |
| | | | | HMGB3, TFRC | |
| FATTY_ACID_METABOLISM | 6.00E−05 | 0.00059 | 155 | ALDH3A2, IDH1, | down |
| | | | | MLYCD, CA2, | |
| | | | | HPGD, ALAD, | |
| | | | | CBR1, CD36, | |
| | | | | HMGCS1, | |
| | | | | ALDH3A1, | |
| | | | | EPHX1, GLUL, | |
| | | | | CYP1A1 | |
| REACTIVE_OXIGEN_SPECIES_PATHWAY | 0.00014 | 0.00119 | 49 | TXNRD1, GCLC, | down |
| | | | | PRDX1, G6PD, | |
| | | | | NQO1, FES, FTL | |
| ADIPOGENESIS | 0.00031 | 0.00224 | 199 | CD36, IDH1, | down |
| | | | | SDPR, FZD4, | |
| | | | | CYP4B1, SSPN, | |
| | | | | SAMM50, | |
| | | | | ABCA1, PRDX3, | |
| | | | | BCL6, ANGPT1, | |
| | | | | PQLC3 | |
| GLYCOLYSIS | 0.00151 | 0.00944 | 200 | EGLN3, AKR1A1, | down |
| | | | | G6PD, DPYSL4, | |
| | | | | STC1, KIF20A, | |
| | | | | IDH1, ELF3, | |
| | | | | MERTK, GCLC | |
| PEROXISOME | 0.00532 | 0.02957 | 102 | MLYCD, | down |
| | | | | SLC27A2, IDH1, | |
| | | | | PRDX1, STS, | |
| | | | | ITGB1BP1, | |
| | | | | SEMA3C | |
| MTORC1_SIGNALING | 0.00709 | 0.03476 | 199 | TFRC, IDH1, | down |
| | | | | EGLN3, PRDX1, | |
| | | | | HMGCS1, | |
| | | | | TXNRD1, G6PD, | |
| | | | | ITGB2, CCNF, | |
| | | | | STC1, GCLC | |
| INTERFERON_GAMMA_RESPONSE | 0.00765 | 0.03476 | 199 | ISG15, IFIT1, | down |
| | | | | RSAD2, MX2, | |
| | | | | OAS3, TNFSF10, | |
| | | | | PSMB8, IFI27, | |
| | | | | HERC6, CMPK2, | |
| | | | | RTP4, TNFAIP2, | |
| | | | | VCAM1, CIITA, | |
| | | | | CFB, IL7, B2M, | |
| | | | | SSPN, IL6, | |
| | | | | VAMP5, ARID5B | |
| INTERFERON_ALPHA_RESPONSE | 0.00968 | 0.04032 | 97 | ISG15, OAS1, | down |
| | | | | RSAD2, IFI27, | |
| | | | | PSMB8, HERC6, | |
| | | | | CMPK2, RTP4, | |
| | | | | B2M, LAMP3, IL7 | |

Gene Set Enrichment Analysis (GSEA) of differentially expressed genes and regulated pathways in DCIS.COM-CRYβB2 cells in comparison to vector. The different pathways, p value, adjusted p value, number of genes differentially expressed, strongly regulated genes and direction of pathway regulation are shown.

### Example 6

CRYβB2 interacts with proteins that regulate translation, cell proliferation and invasion.

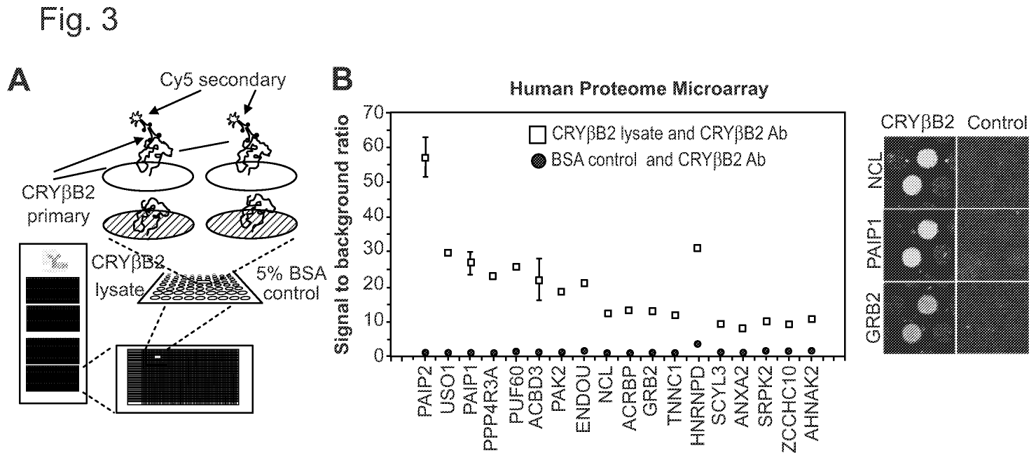

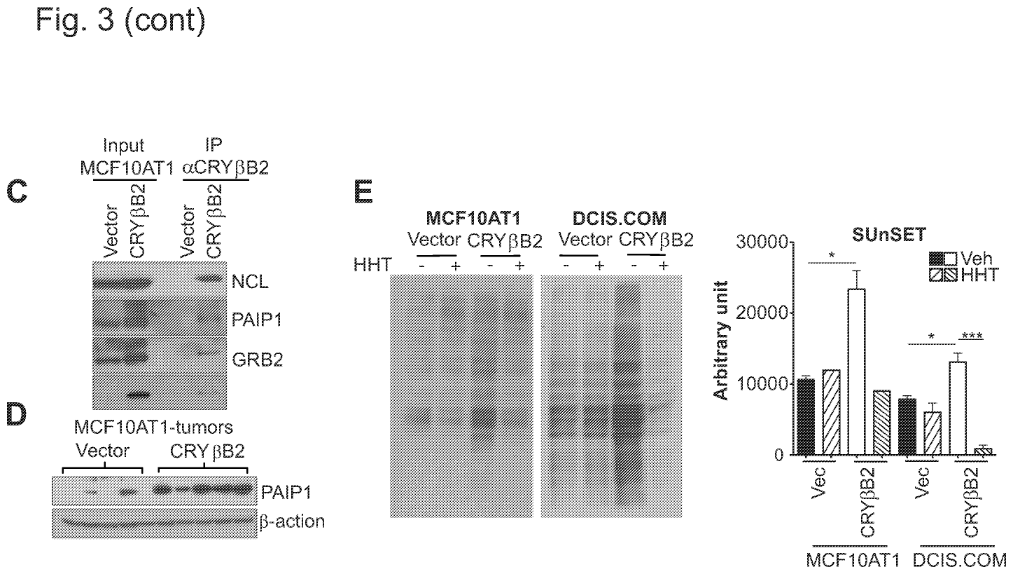

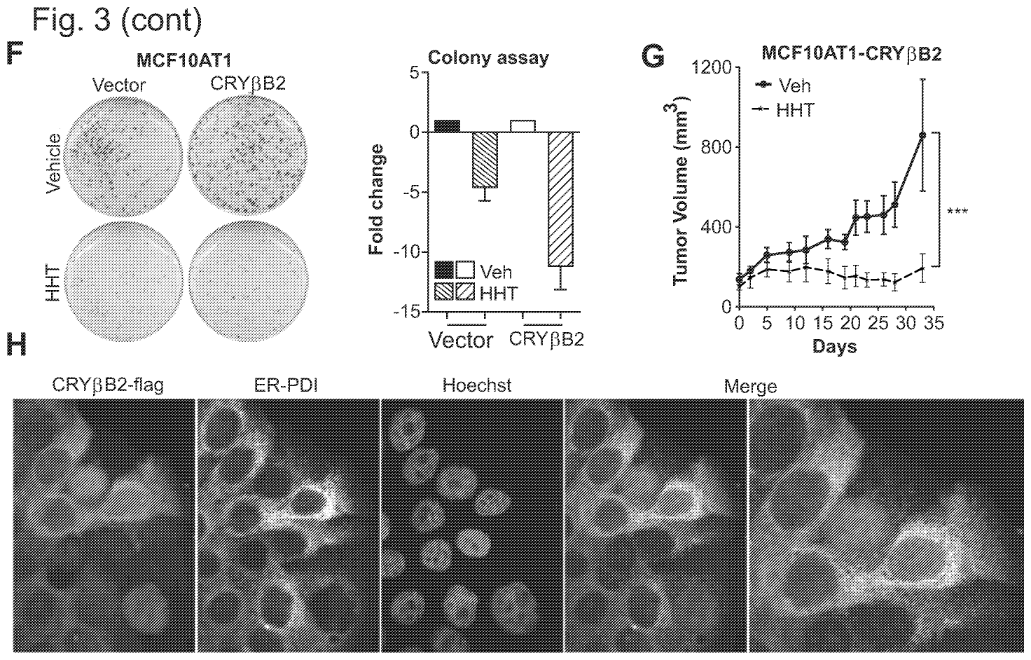

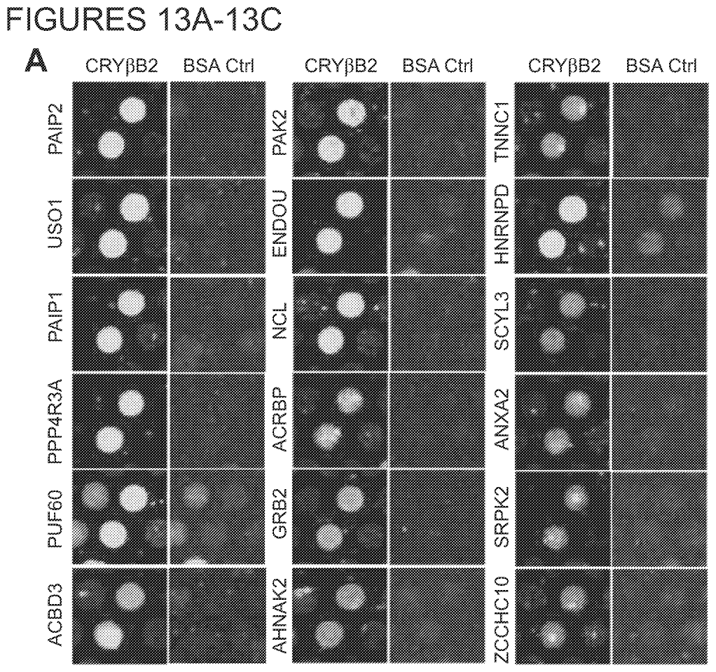

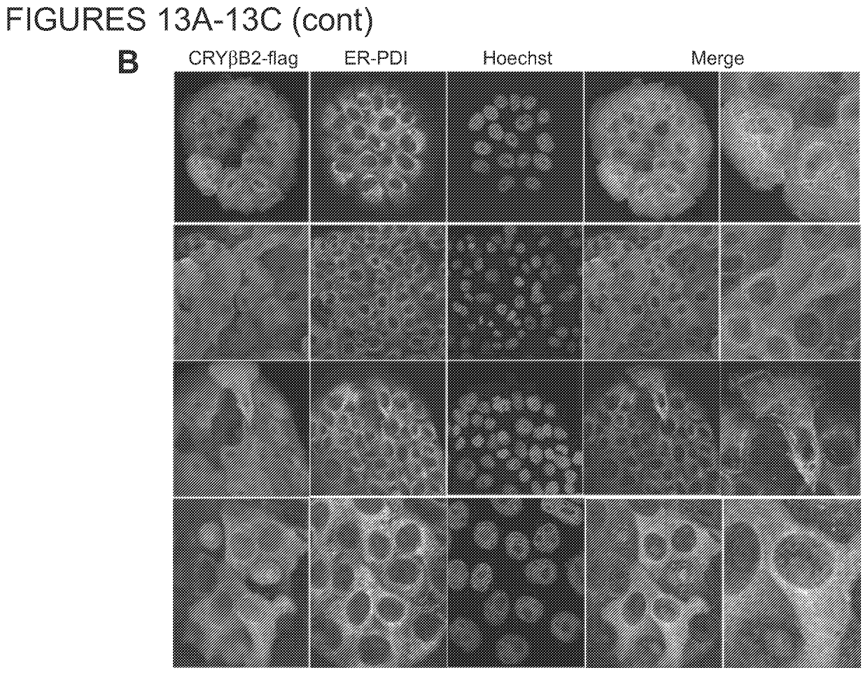

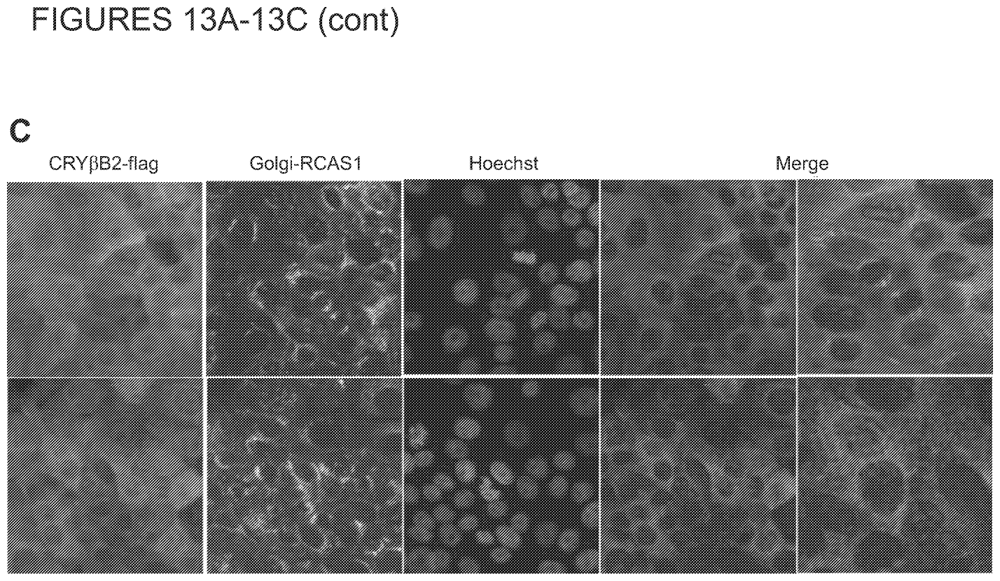

In order to identify CRYβB2-interacting proteins and decipher additional mechanisms of CRYβB2-induction of malignancy, we screened the human proteome microarray. CRYβB2 binding to immobilized proteins was detected with a CRYβB2—specific antibody and developed with a fluorescent-labeled secondary antibody (FIG. 3A). Several CRYβB2-interacting proteins are known to be involved in control of translation, such as PAIP1, PAIP2, USO1, PUF60, ENDOU, nucleolin, ACBD3; cell death PAK2; DNA damage PPP4R3A; DNA repair HNRNPD; self-renewal ACBD3; and proliferation USO1, GRB2, ENDOU and ANXA2 (FIG. 3B, FIG. 13A and Table 4). We also observed CRYβB2 interaction with several proteins involved in tumor cell invasion and metastasis (Table 4). Using co-immunoprecipitation, we validated the binding of CRYβB2 to nucleolin, PAIP1 and GRB2 using lysates of MCF10AT1-CRYβB2 cells (FIG. 3C). The specificity for CRYβB2 binding was supported by the fact that these proteins were not immunoprecipitated (IP) using MCF10AT1-vector cell-lysate that lacked CRYβB2 expression (FIG. 3C). MCF10AT1-CRYβB2 tumors showed increased levels of poly(A) binding protein interacting protein 1 (PAIP1), which regulates initiation of translation (FIG. 3D).

Since CRYβB2 associates with a number of proteins that regulate protein translation, we investigated if it can increase total protein synthesis using a puromycin-based pulse assay, SUnSET, as described (38). MCF10AT1 and DCIS.COM cells overexpressing CRYβB2 showed an increase in incorporation of puromycin into nascent proteins, detected by an increase intensity of the smear in immunoblots using anti-puromycin antibody (FIG. 3E). The inhibitor of protein translation, homoharringtonine (HHT), was more effective in decreasing protein synthesis (FIG. 3E) and cell proliferation (FIG. 3F) of MCF10AT1-CRYβB2 in comparison to vector control cells. Further, HHT treatment of MCF10AT1-CRYβB2 tumors resulted in significant inhibition of growth (FIG. 3G). These data showed that CRYβB2-regulation of protein synthesis is important for tumor growth.

TABLE 4
CRYβB2-interactome.

| | | BSA | | |
|---|---|---|---|---|
| | CRYBB2 | Ctrl | Ratio | |
| Protein | SNB | SNB | SNBs | Function |
| PAIP2 | 57.2 ± 5.6 | 1.2 ± 0.1 | 47.0 | Protein synthesis (39) |
| USO1 | 29.9 ± 0.5 | 1.2 ± 0.0 | 25.3 | Endoplasmic reticulum-Golgi trafficking (40) |
| PAIP1 | 27.0 ± 3.2 | 1.1 ± 0.1 | 24.2 | Protein synthesis (41) |
| PPP4R3A | 23.4 ± 0.2 | 1.2 ± 0.1 | 18.9 | DNA repair (42) |
| PUF60 | 25.8 ± 0.2 | 1.6 ± 0.0 | 16.6 | Poly(U) Binding splicing factor (43) |
| ACBD3 | 22.2 ± 6.0 | 1.4 ± 0.1 | 16.2 | Maintenance of Golgi structure (44); stemness (45) |
| PAK2 | 18.8 ± 0.3 | 1.3 ± 0.0 | 15.0 | Cell death (46) |
| ENDOU/Nsp15 | 21.3 ± 0.0 | 1.8 ± 0.1 | 12.1 | Poly(U)- endoribonuclease, viral replication (47) |
| NCL | 12.6 ± 1.1 | 1.1 ± 0.1 | 11.8 | Synthesis and maturation of ribosomes (48) |
| ACRBP/OY-TES1 | 13.1 ± 0.4 | 1.1 ± 0.2 | 11.7 | Cell proliferation and migration (49) |
| GRB2 | 13.0 ± 0.0 | 1.2 ± 0.0 | 11.3 | Cell growth, proliferation and metabolism (50) |
| TNNC1 | 12.2 ± 0.5 | 1.2 ± 0.1 | 9.9 | Metastasis (51) |
| HNRNPD | 31.5 ± 0.5 | 3.8 ± 0.5 | 8.3 | RNA binding protein and DNA repair (52) |
| SCYL3/PACE-1 | 9.4 ± 0.7 | 1.4 ± 0.1 | 6.8 | Ezrin-binding protein (53) |
| ANXA2 | 13.9 ± 0.3 | 2.4 ± 0.0 | 5.8 | Cell proliferation and migration (54) |
| SRPK2 | 10.4 ± 0.3 | 1.6 ± 0.1 | 6.6 | Tumor growth and metastasis (55) |
| ZCCHC10 | 9.4 ± 0.0 | 1.5 ± 0.1 | 6.1 | Reduction of tumor growth and metastasis (56) |
| AHNAK2 | 10.3 ± 0.4 | 1.8 ± 0.1 | 5.9 | Nucleoprotein- EMT and stemness (57) |
| NUTM2G | 12.0 ± 0.5 | 2.1 ± 0.1 | 5.7 | ND |
| NDRG4 | 7.1 ± 0.0 | 1.2 ± 0.2 | 5.9 | Reduction of tumor growth and metastasis (58) |
| YWHAZ/14-3-3z | 8.6 ± 8.2 | 1.5 ± 0.1 | 5.8 | Metastasis (59) |
| DBNL/HIP-55 | 6.8 ± 0.1 | 1.2 ± 0.0 | 5.7 | Cell proliferation and migration (60) |
| TMEM44 | 8.0 ± 0.7 | 1.4 ± 0.1 | 5.7 | ND |
| ANXA6 | 6.3 ± 0.0 | 1.1 ± 0.1 | 5.5 | Breast cancer metastasis (61) |
| H1F0/H1.0 | 6.7 ± 0.0 | 1.2 ± 0.1 | 5.5 | Differentiation (62) |
| ANXA1 | 7.6 ± 0.3 | 1.4 ± 0.1 | 5.4 | Cell proliferation and metastasis (63) |
| BROX | 5.7 ± 4.7 | 1.1 ± 0.0 | 5.4 | ND |
| DSTYK | 8.7 ± 1.7 | 1.7 ± 0.0 | 5.3 | ND |
| ANXA5 | 6.9 ± 0.3 | 1.3 ± 0.1 | 5.3 | Tumor progression and metastasis (64) |
| EPB41L2/Band 4.1 | 6.9 ± 0.7 | 1.3 ± 0.2 | 5.2 | Cell spreading (65), survival and proliferation (66) |
| WARS/TrpRS | 7.0 ± 0.5 | 1.4 ± 0.1 | 5.1 | Tryptophan metabolism and protein synthesis (67) |
| ZSCAN5A/ZNF495 | 6.5 ± 0.2 | 1.3 ± 0.0 | 5.0 | Cell cycle progression (68) |

CRYβB2-interacting proteins identified by HuProt™ Human Proteome Microarray using protein lysate of MCF10AT1-CRYβB2 cells (CRYβB2) or BSA control (BSA Ctrl). The ratio of CRYβB2 binding normalized by BSA binding following detection with CRYβB2 primary and Cy5—secondary antibodies is shown. Proteins with a CRYβB2 binding and a ratio above 5 and their functions are listed. SNB:Signal and background ratio; SD:standard deviation; ND:not determined.

### Example 7

CRYβB2 is a shuttling protein and associates with the endoplasmic reticulum.

We investigated the localization of CRYβB2. We observed that CRYβB2 is a nucleocytoplasmic shuttling protein and is localized in either the cytoplasm or the nucleus (FIG. 3H). Since CRYβB2 associated with proteins that regulate translation and trafficking of proteins from endoplasmic reticulum to Golgi, like USO1 and ACBD3 (FIG. 3B and Table 4), we determined if CRYβB2 localizes within these organelles. Confocal microscopy of labeled cells revealed that CRYβB2 associates with an endoplasmic reticulum marker, the PDI protein (FIG. 3H and FIG. 13B). On the other hand, CRYβB2 did not associate with RCAS1, a Golgi marker (FIG. 13C). Confocal microscopy and analysis of the mitochondrial fraction revealed that even though These results suggest that in the cytoplasm CRYβB2 associates with endoplasmic reticulum proteins and trafficking of proteins from the endoplasmic reticulum to Golgi may have a role in CRYβB2—mediated promotion of malignancy.

### Example 8

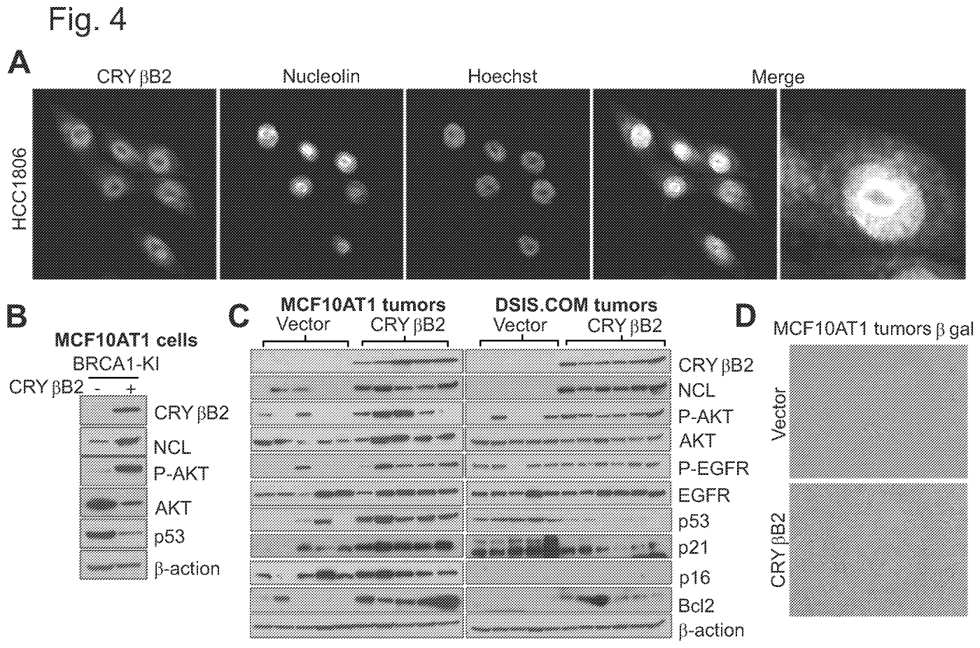

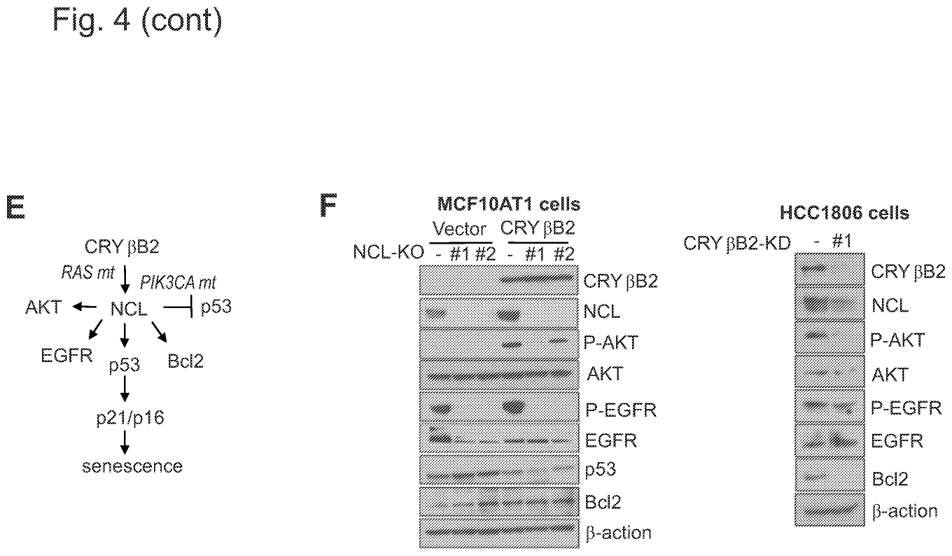

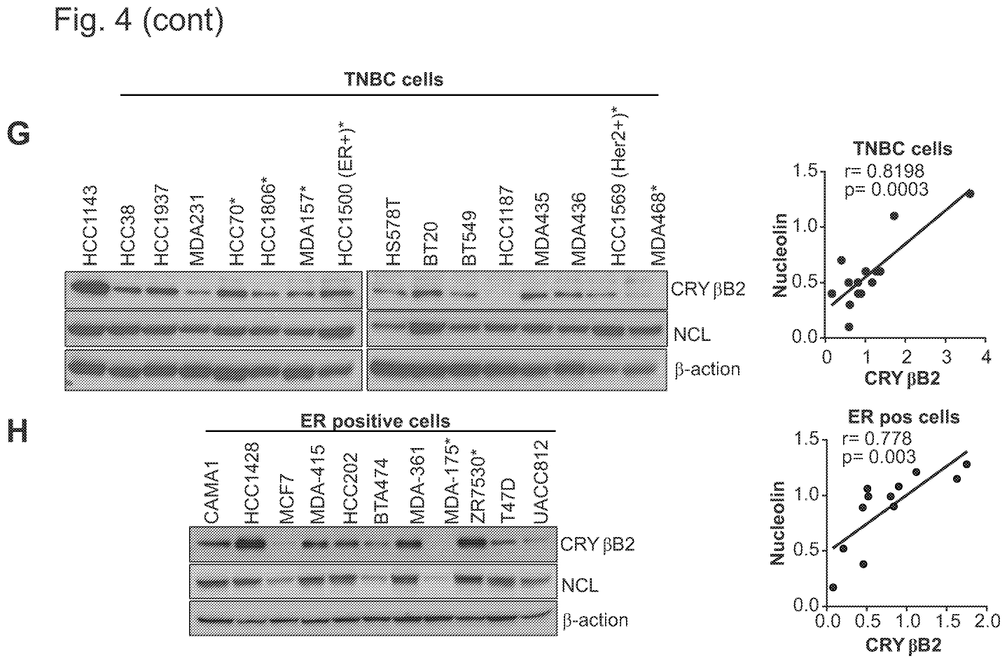

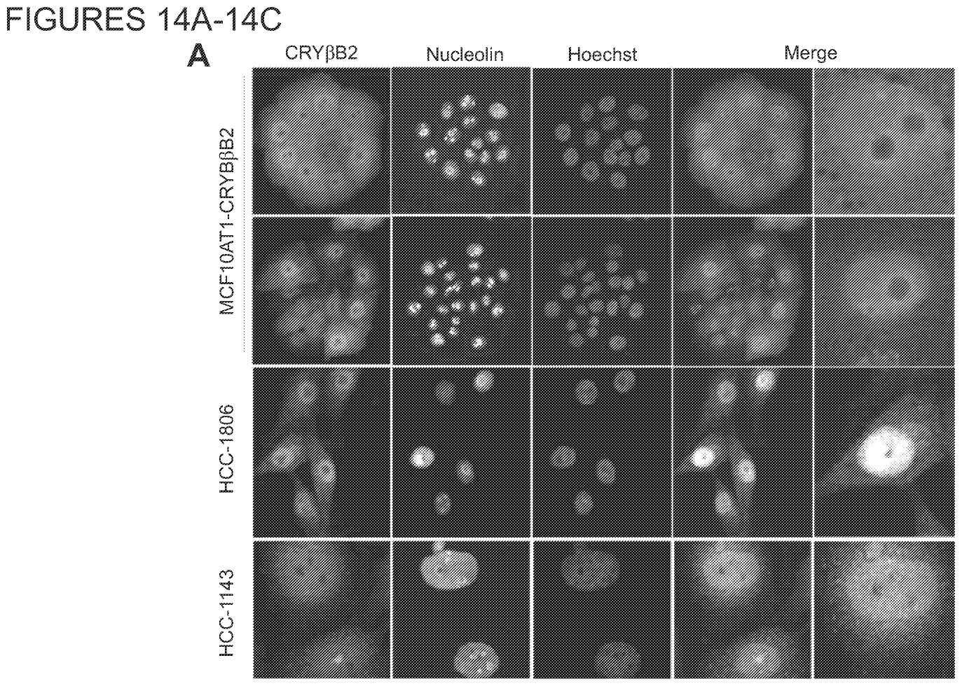

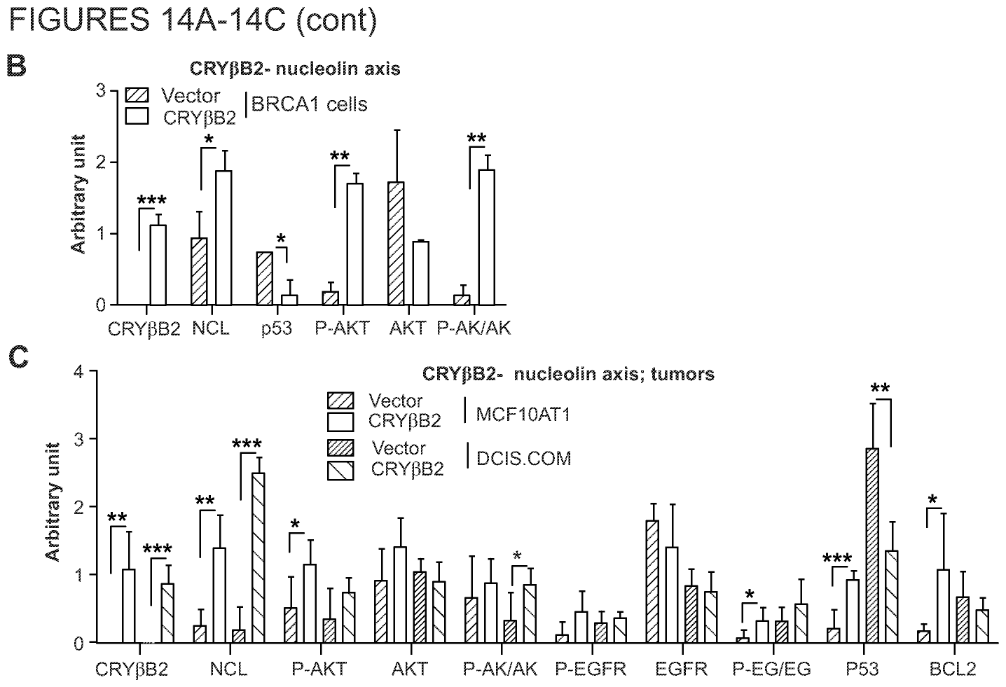

CRYβB2 regulates nucleolin expression and function. Nucleolin is a multifunctional protein that is mainly localized in the nucleolus, where it regulates protein synthesis and cell proliferation (69). CRYβB2 co-localizes with nucleolin in the nucleus (FIG. 4A and FIG. 14A). In addition, CRYβB2 expression significantly increased the protein levels of nucleolin and activation of its associated proteins, including AKT and EGFR and the pro-survival Bcl2 protein in premalignant MCF10A-BRCA1-185delAG knock-in (KI) (70) (FIG. 4B and FIG. 14B), and MCF10AT1 and DCIS.COM tumors (FIG. 4C and FIG. 14C). With the exception of MCF10AT1 tumors, CRYβB2 expression resulted in decreased p53 levels (FIG. 4B, FIG. 4C, FIG. 14B and FIG. 14C). Overexpression of CRYβB2 in low malignant MCF10AT1 cells, which has single HRAS mutation, resulted in activation of senescence proteins, such as p53, p21 and p16, in tumors (FIG. 4C). Accordingly, MCF10AT1-CRYβB2 tumors showed increase in β-galactosidase staining, a marker of senescence (FIG. 4D). These data show that in low malignant cells, with single mutation in HRAS, CRYβB2 increases p53 and induces senescence (FIG. 4E). Oncogenic RAS typically triggers cellular senescence, a state of irreversible cell growth arrest (71). However, senescence can also promote cancer development by altering the cellular microenvironment through a senescence-associated secretory phenotype (SASP) (72). On contrary, in more malignant cells, with mutations in both MAPK and PIK3CA, CRYβB2 decreases p53 (FIG. 4E). Detailed analysis showed that knockout of nucleolin impaired AKT and EGFR activation in MCF10AT1 cells expressing CRYβB2 (FIG. 4F). We observed that CRYβB2 protein levels also correlated with nucleolin expression in TNBC (FIG. 4G) and ER⁺ (FIG. 4H) cell lines. These data show that CRYβB2 increases nucleolin-related pathways in breast cancer cells.

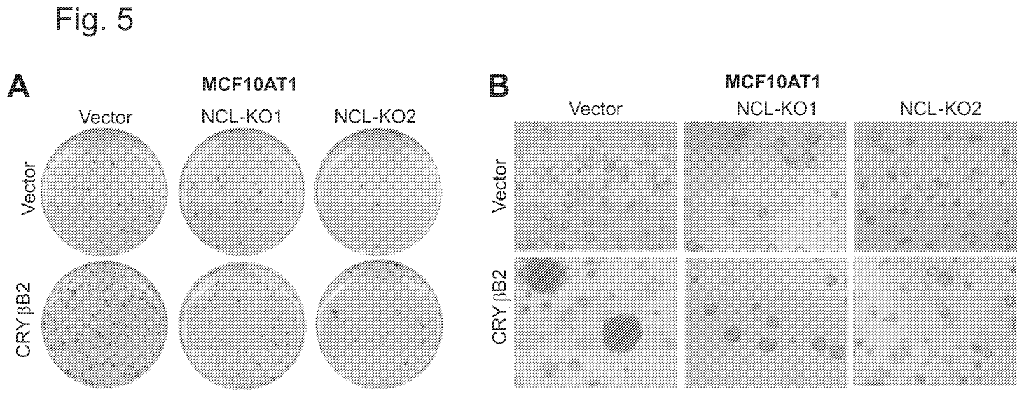

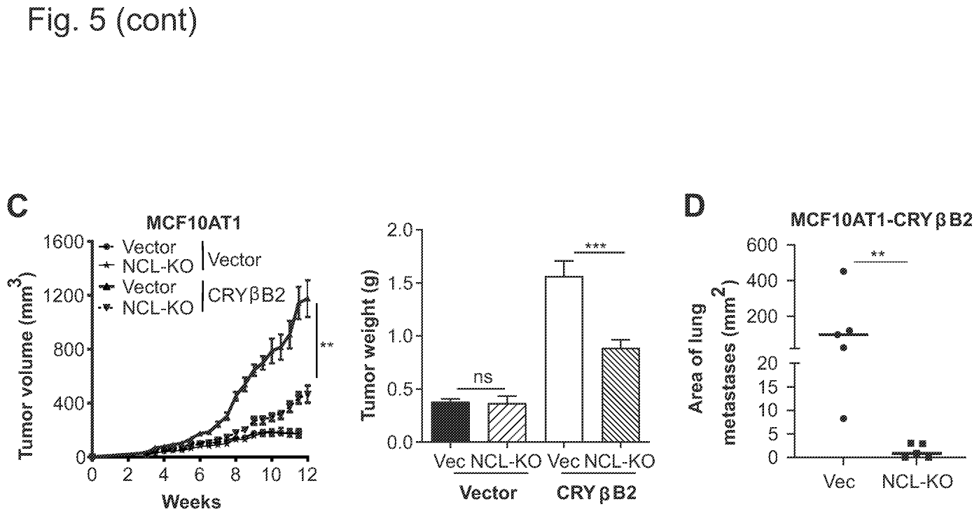

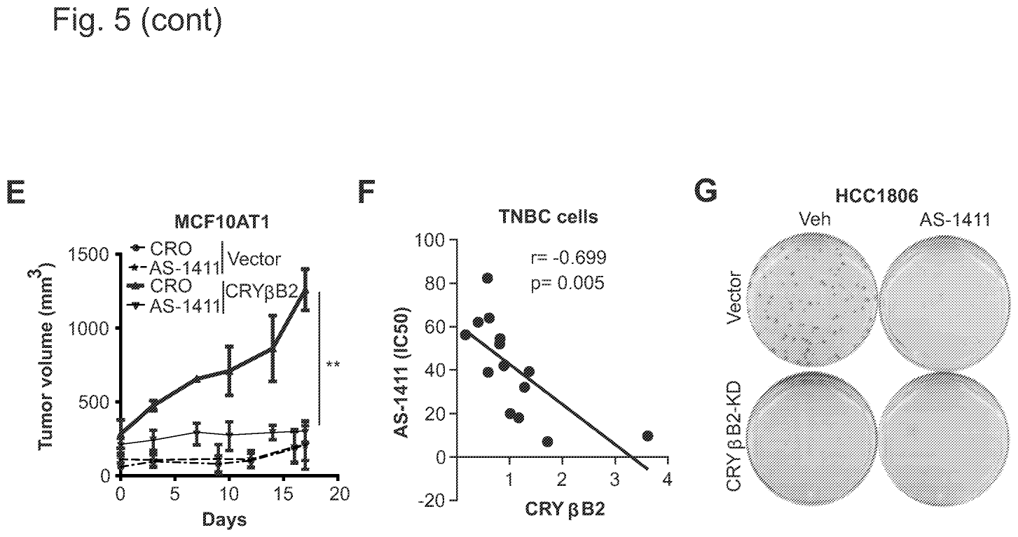

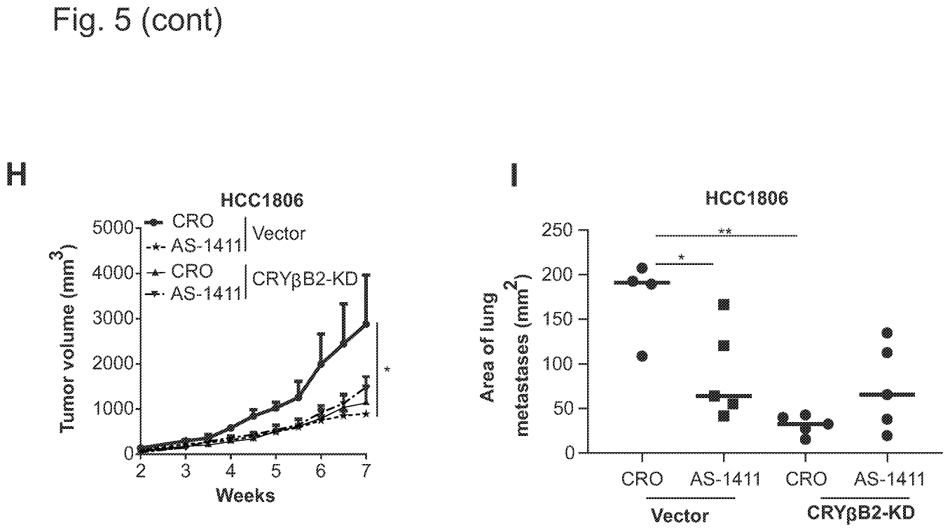

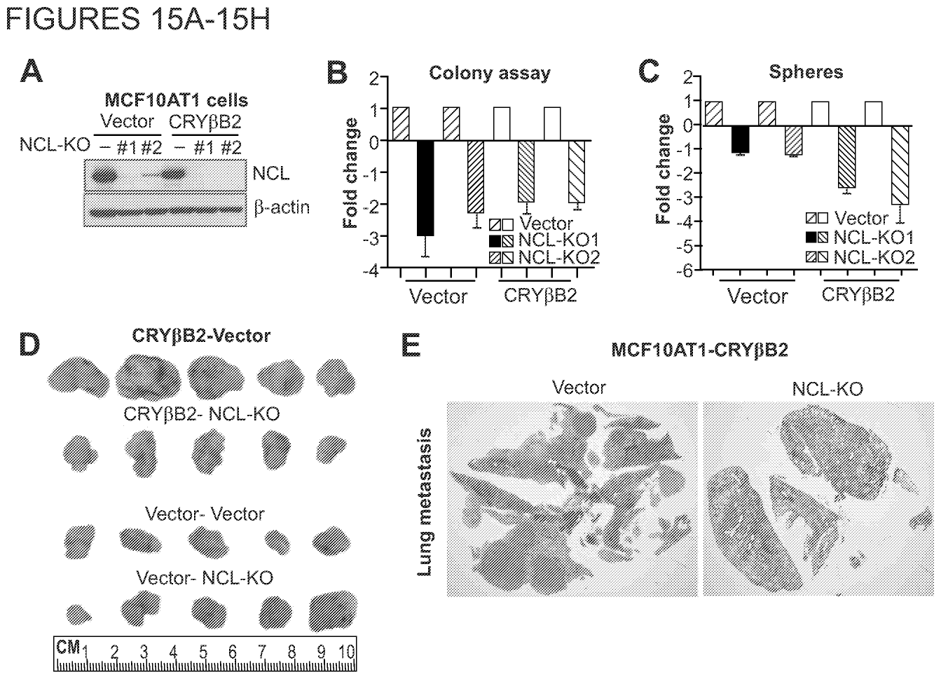

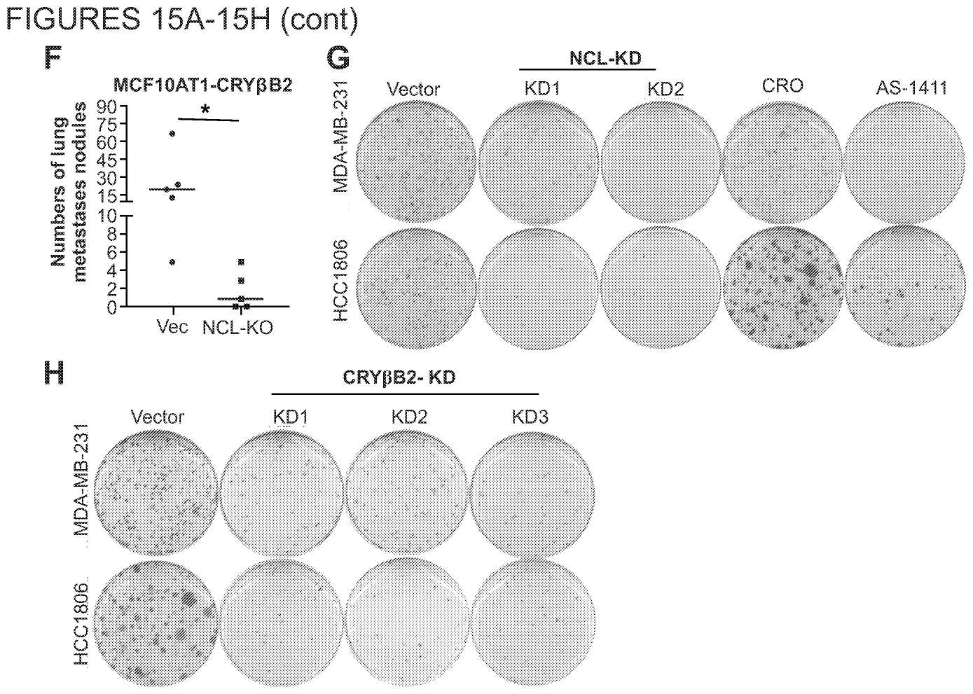

Nucleolin has been previously described to play a role in tumor cell proliferation (69), metastasis (73) and stem cell maintenance (74-76). To address if nucleolin is involved in the CRYβB2—induction of malignancy we depleted nucleolin from MCF10AT1-vector and -CRYβB2 overexpressing cells (FIG. 15A). Knockout of nucleolin significantly decreased proliferation (FIG. 5A and FIG. 15B), sphere formation (FIG. 5B and FIG. 15C), and tumor size and weight (FIG. 5C and FIG. 15D) of MCF10AT1-CRYβB2 cells. Interestingly, nucleolin deficiency had a smaller effect on sphere formation by MCF10AT1-vector cells (FIG. 5B and FIG. 15C) and no effect on tumor formation by these cells (FIG. 5C and FIG. 15D). Importantly, MCF10AT1-CRYβB2 tumors that are nucleolin-deficient showed significantly lower incidence and size of lung metastases (FIG. 5D, FIG. 15E and FIG. 15F). In line with these observations, the nucleolin aptamer AS-1411 inhibited the growth of CRYβB2 tumors and had no effect in tumors lacking CRYβB2 (FIG. 5E). In addition tolow-malignant tumors, we investigate the role of CRYβB2 in TNBC growth and response to treatment. We observed that CRYβB2 expression inversely correlated to AS-1411 IC50 in several TNBC cells (FIG. 5F), suggesting that CRYβB2 sensitizes TNBC cells to nucleolin inhibitors. Knockdown of CRYβB2 significantly impaired the growth of TNBC cells and response to AS-1411 in cells (FIG. 5G, FIG. 15G and FIG. 15H) and tumors (FIG. 5H). The decrease of CRYβB2 levels significantly inhibited formation of metastases (FIG. 5I). Higher levels of CRYβB2 in TNBC cells resulted in AS-1411-mediated decrease of metastases (FIG. 5I). These results provide evidence that nucleolin is, largely, a mediator of CRYβB2-related induction of tumor cell proliferation, metastasis and stem cell function. CRYβB2 can be used as a biomarker of response to nucleolin inhibitors.

### Example 9

CRYβB2 associates with poor TNBC outcome in AA women.

We observed that CRYβB2 is overexpressed in ER⁻ tumors from AA patients (FIG. 1B) and promoted xenograft tumor growth (FIG. 1D). Therefore, we investigated whether CRYβB2 expression may correlate with AA-TNBC patient survival.

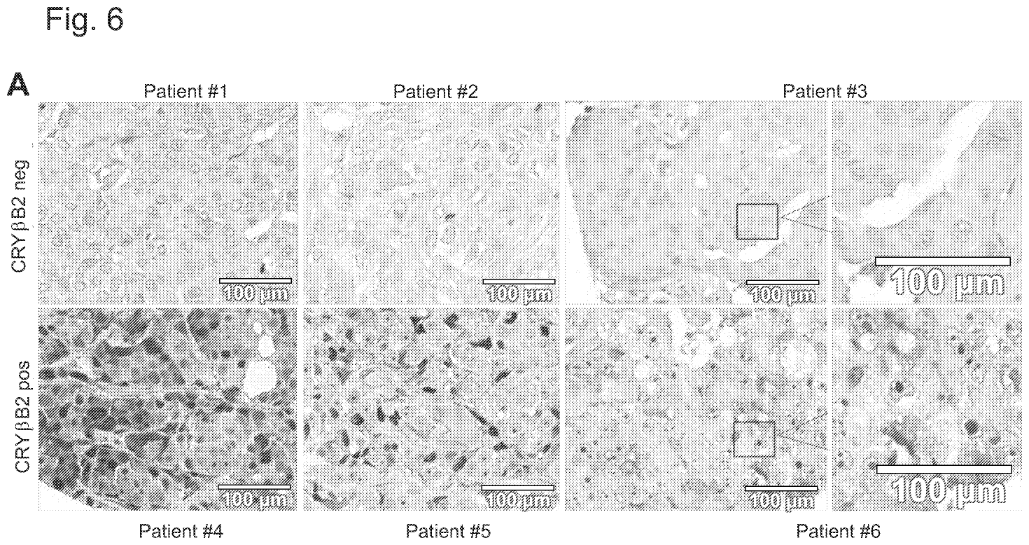

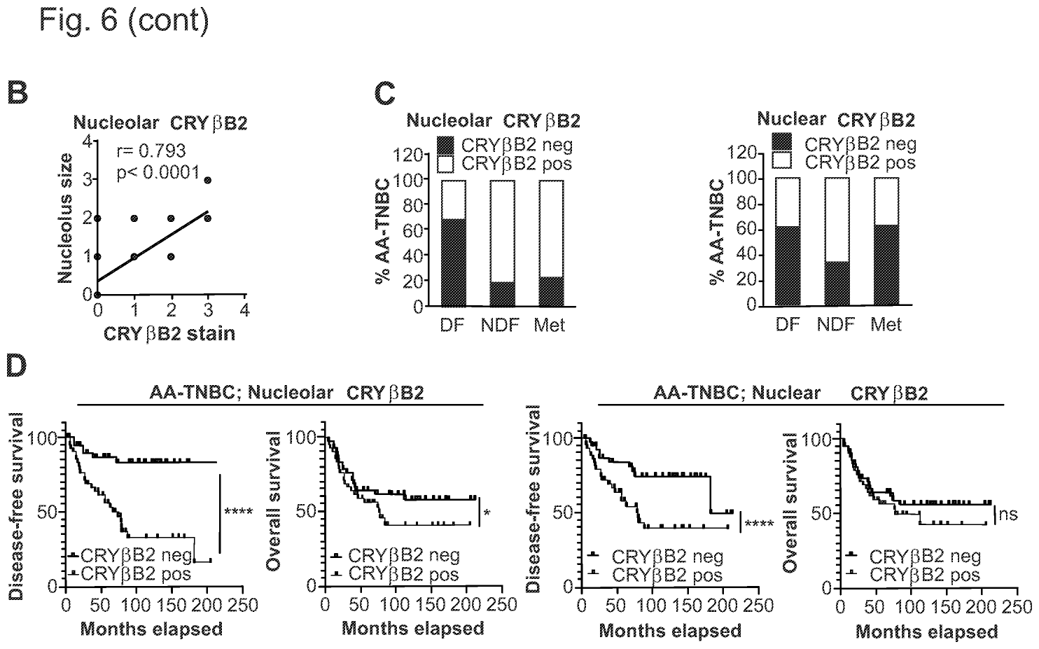

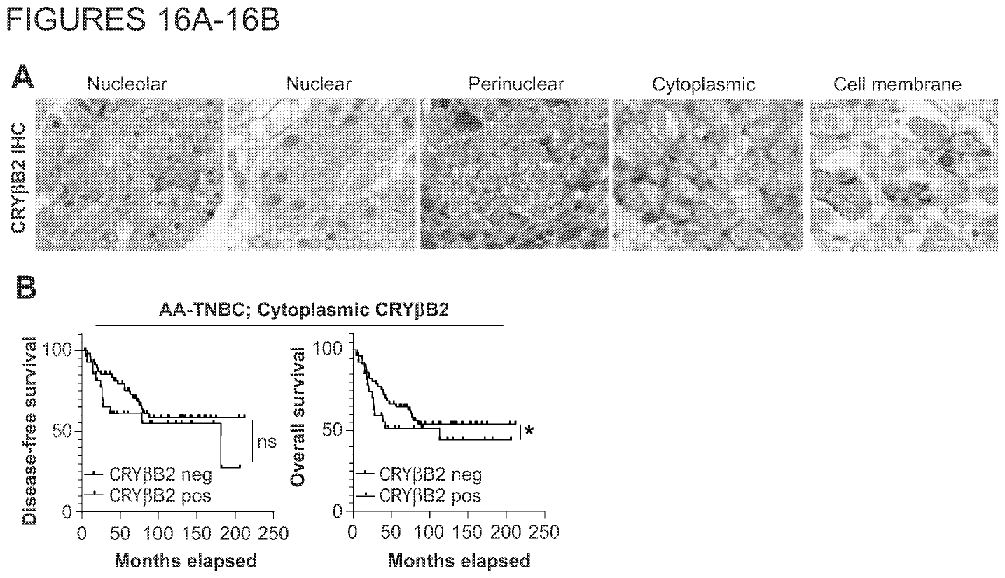

Consistent with the nucleocytoplasmic trafficking properties of CRYβB2, we observed various localization patterns of CRYβB2 expression in TNBC patients (FIG. 6A). CRYβB2 was expressed mainly in the nucleus, nucleolus and cytoplasm and less often on the surface of tumor cells (FIG. 6A and FIG. 16A). Furthermore, and consistent with our findings in tumor xenografts (FIG. 2A), nucleolar CRYβB2 expression correlated with an increase in nucleolar size (FIG. 6A and FIG. 6B). We observed nucleolar CRYβB2 expression in 81% (95% CI: 64%-93%) of the tumors in AA TNBC patients who were never disease-free (n=32) and in 77% (95% CI: 46%-95%) of the metastasis (n=13) (FIG. 6C). In contrast, most tumors from TNBC patients who remained disease-free (n=55) lacked nucleolar CRYβB2 expression (n=38 or 69%; 95% CI: 55%-81%) (FIG. 6C). We also observed nuclear CRYβB2 expression in 67% (95% CI: 48%-82%) of the tumors in AA TNBC patients who were never disease-free (n=33) but lack of nuclear CRYβB2 expression in most disease-free patients (34 out of 56 or 61%; 95% CI: 47%-74%) (FIG. 6C). Importantly, the presence of nucleolar and nuclear, but not cytoplasmic, CRYβB2 expression in TNBC associated with a significant decrease in disease-free (n=86, both P<0.0001) and overall survival (n=82, nucleolar P=0.0167, nuclear P=0.2485) among AA women with TNBC (FIG. 6D and FIG. 16B). Collectively, these data show that CRYβB2 is associated with an increase in nucleolar size and poor prognosis in AA-TNBC patients.

### Example 10

CRYβB2 activates CDK4/pRb pathway in premalignant cells and breast tumors.

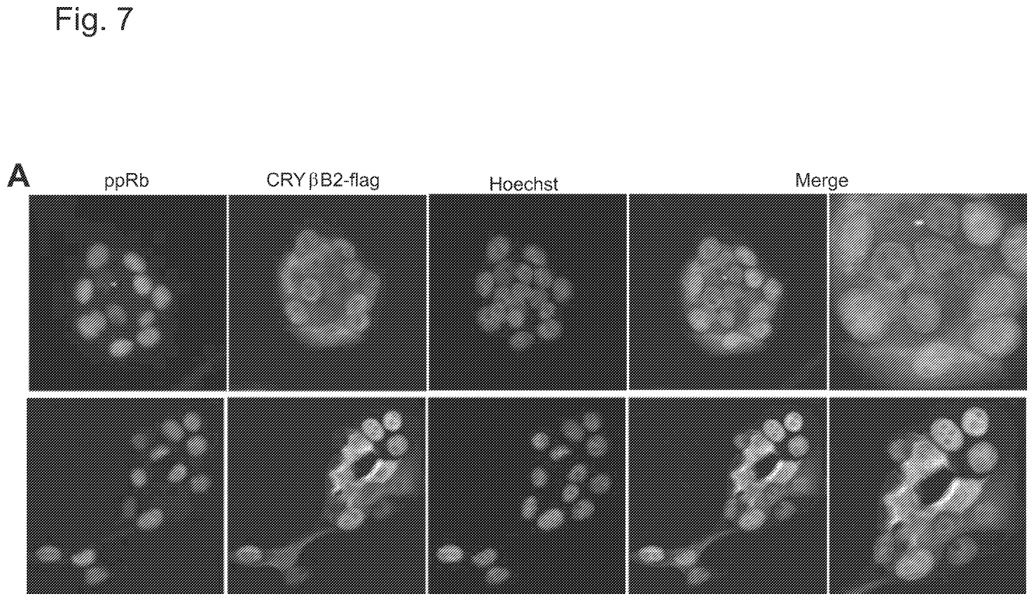

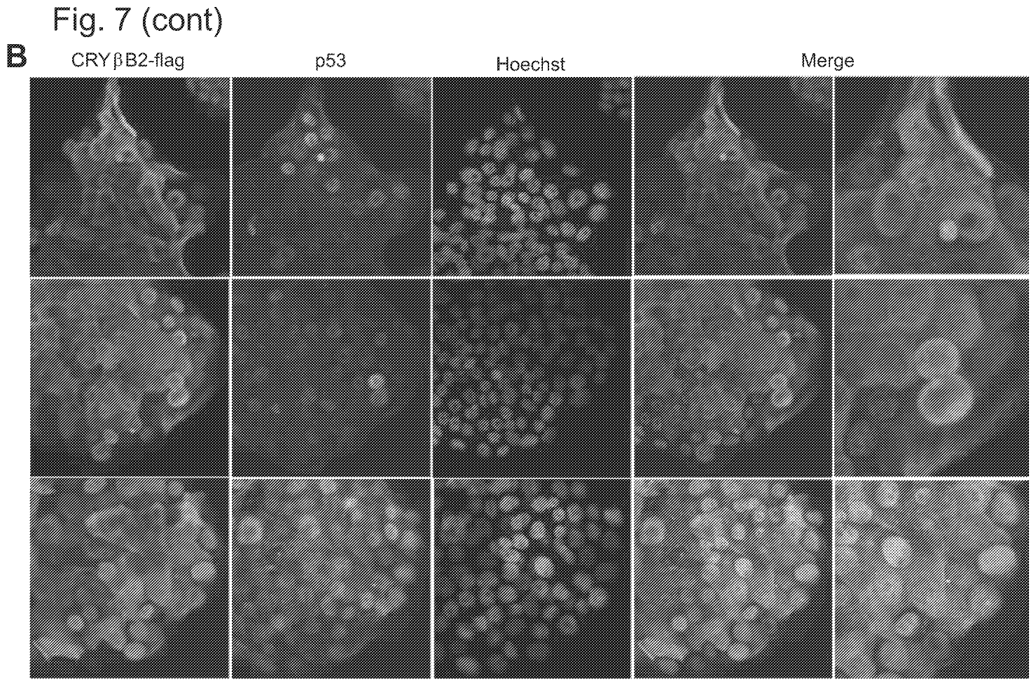

Nucleolin also induces malignancy by regulation of cell cycle (73). It associates with the tumor suppressors, retinoblastoma protein (pRb) (77) and p53 (78). Nucleolin is involved in post-transcriptional inhibition of the p53 (78). Since CRYβB2 induced nucleolin (FIG. 4C), we asked whether p53-dependent regulation of the cell cycle is a target of CRYβB2-induced malignancy. CRYβB2 co-localized within ppRb (FIG. 7A) and p53 (FIG. 7B) in the nucleus of MCF10AT1 cells.

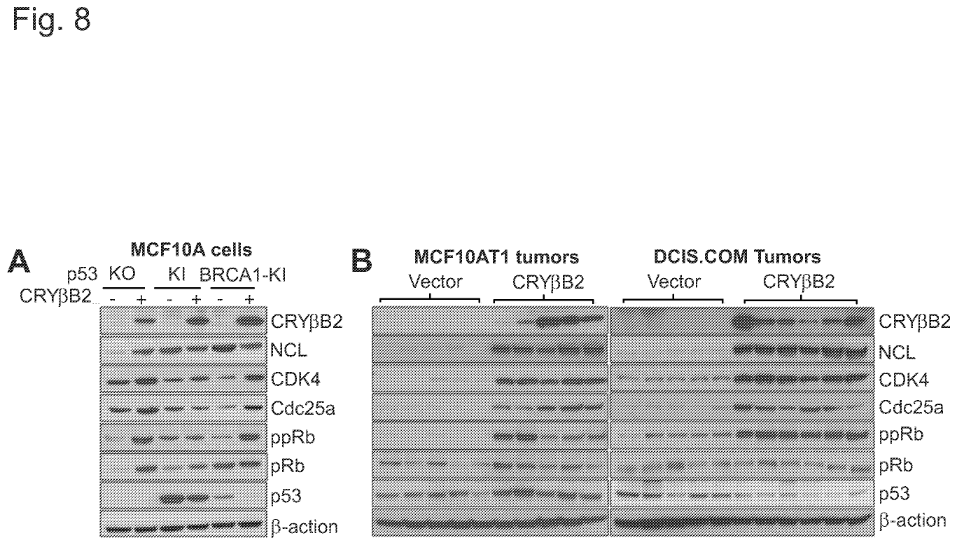

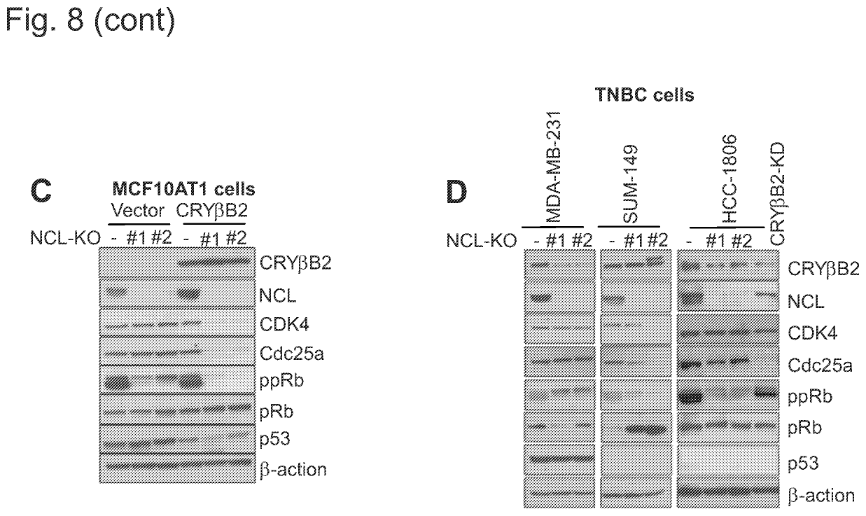

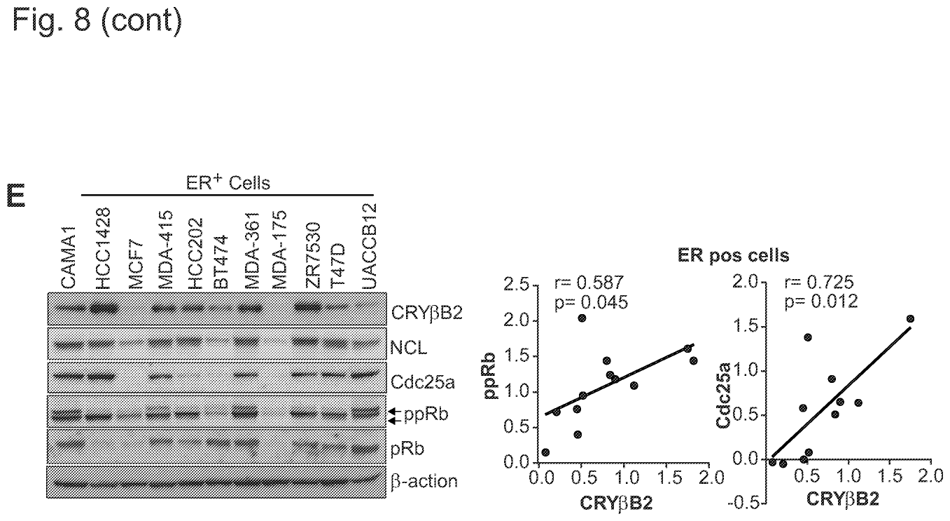

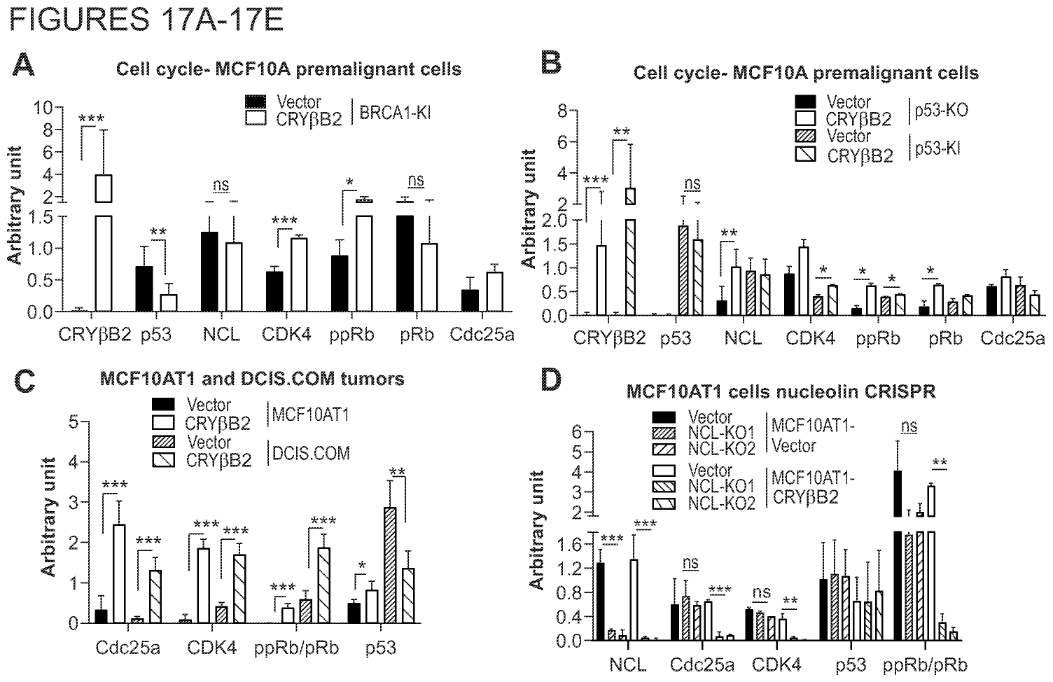

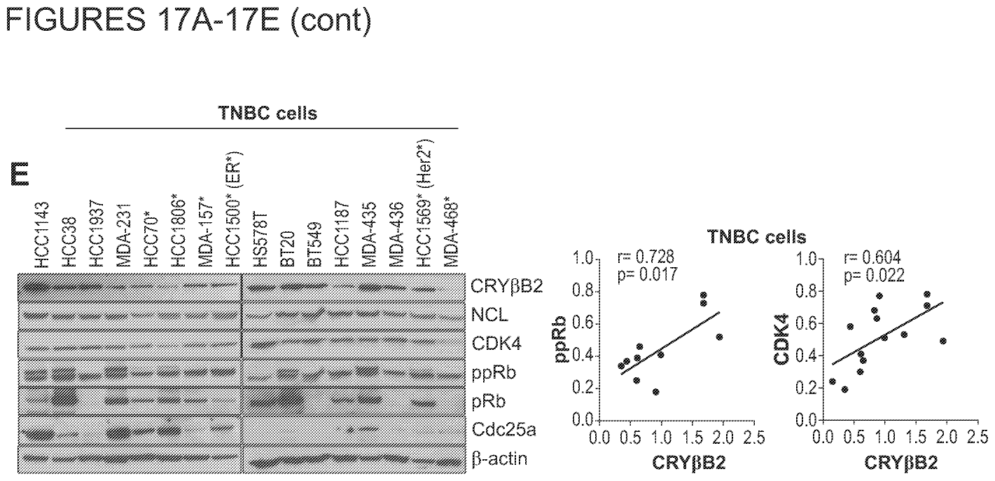

CRYβB2 overexpression resulted in p53 downregulation in premalignant MCF10A-BRCA1-185delAG mutant (70) (FIG. 8A, FIG. 17A and FIG. 17B), DCIS.COM tumors (FIG. 8B and FIG. 17C) and MCF10AT1 cells (FIG. 8C and FIG. 17D). The expression of other proteins related to transition of cells from G1 to S phase of the cell cycle, including Cdc25A, CDK4 and phosphorylated pRb (ppRb) were increased by overexpression of CRYβB2 in premalignant MCF10A-BRCA1-185delAG (70), -p53-R248W knockin (79), and -p53 null cells (80) (FIG. 8A, FIG. 17A and FIG. 17B), and MCF10AT1 and DCIS.COM tumors (FIG. 8B and FIG. 17C). The activation of CDK4/pRb pathway by CRYβB2 was more significant in p53 null than p53 mutant cells (FIG. 8A, FIG. 17A and FIG. 17B), suggesting that functional p53 may restrict CRYβB2-induced activation of cell cycle progression.

Detailed analysis showed that nucleolin deficiency impaired the expression of these proteins related to cell cycle progression in MCF10AT1-CRYβB2 cells (FIG. 8C and FIG. 17D). There was no effect on the expression of cell cycle proteins, except a slight decrease in ppRb, by nucleolin deficiency in MCF10AT1-vector cells, which lack CRYβB2 expression (FIG. 8C and FIG. 17D). Nucleolin deficiency and CRYβB2 knockdown in TNBC cells decreased CRYβB2, as shown in FIG. 8D. However a subsequent decrease of cell cycle proteins were exclusively observed in cells that express lower levels of p53, such as SUM-149 and HCC-1806 (FIG. 8D). Decrease of CRYβB2 in MDA-MB-231 cells, which express higher levels of p53, did not result in a decrease of cell cycle proteins (FIG. 8D). Lastly, CRYβB2 was found to activate the CDK4/pRb pathway in ER⁺ (FIG. 8E) and TNBC cells (FIG. 17E).

### Example 11

CRYβB2 induces cell cycle progression and sensitize tumors to CDK4 inhibitors.

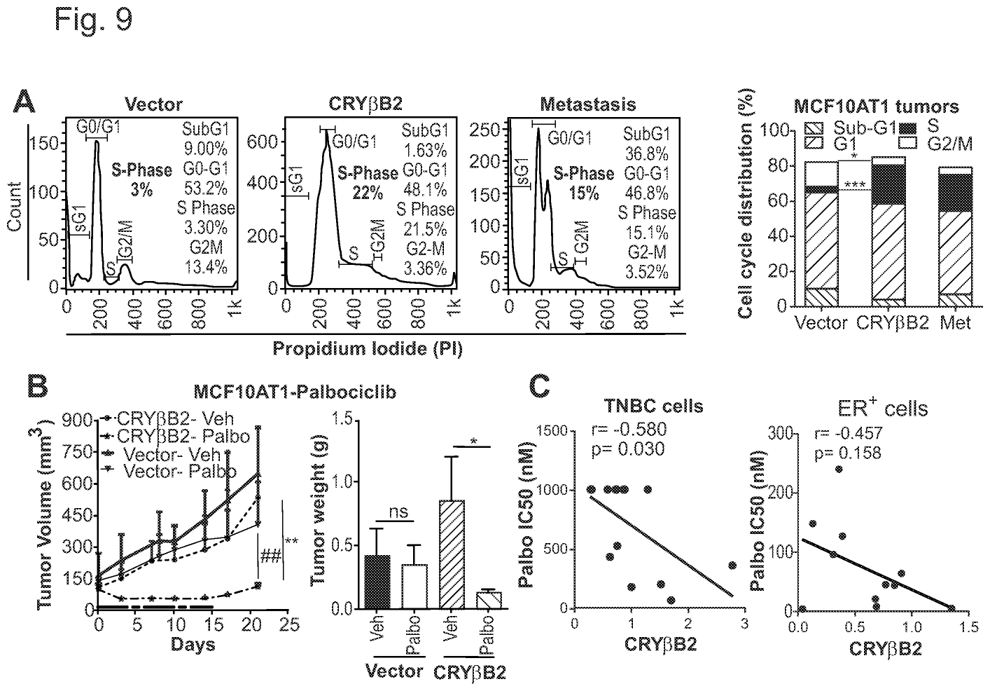

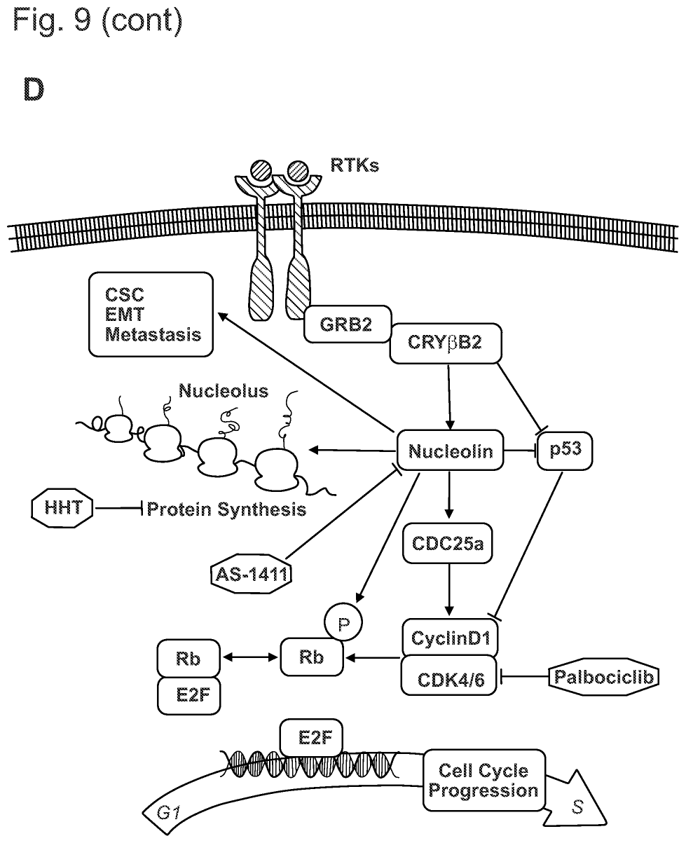

To further assess the role of CRYβB2 in tumor growth, we obtained MCF10AT1-CRYβB2 cells from primary tumor xenografts and associated metastases and found that these cells have an increased number of cells in S phase of the cell cycle compared to cells from control tumors (FIG. 9A).

Importantly, the growth of MCF10AT1-CRYβB2 tumors was significantly decreased by treatment of tumor-bearing mice with the CDK4 inhibitor, palbociclib (FIG. 9B), while palbociclib had no effect on the size of MCF10AT1-vector tumors, which lack CRYβB2 expression (FIG. 9B). Moreover, CRYβB2 protein expression inversely correlated with the reported palbociclib IC₅₀ (21) in both TNBC and ER⁺ cell lines (FIG. 9C). These data strongly suggest that CRYβB2 expression in TNBC and ER⁺ tumors may enhance sensitivity to CDK4 inhibitors (FIG. 9D).

### Example 12

CRYβB2 and ppRb expression are associated with poor TNBC outcome in AA women.

We observed that CRYβB2 promotes xenograft tumor growth and is also overexpressed in ER⁻ tumors from AA patients (FIG. 1B). Thus, we asked whether CRYβB2 and ppRb expression may associate with AA-TNBC patient survival.

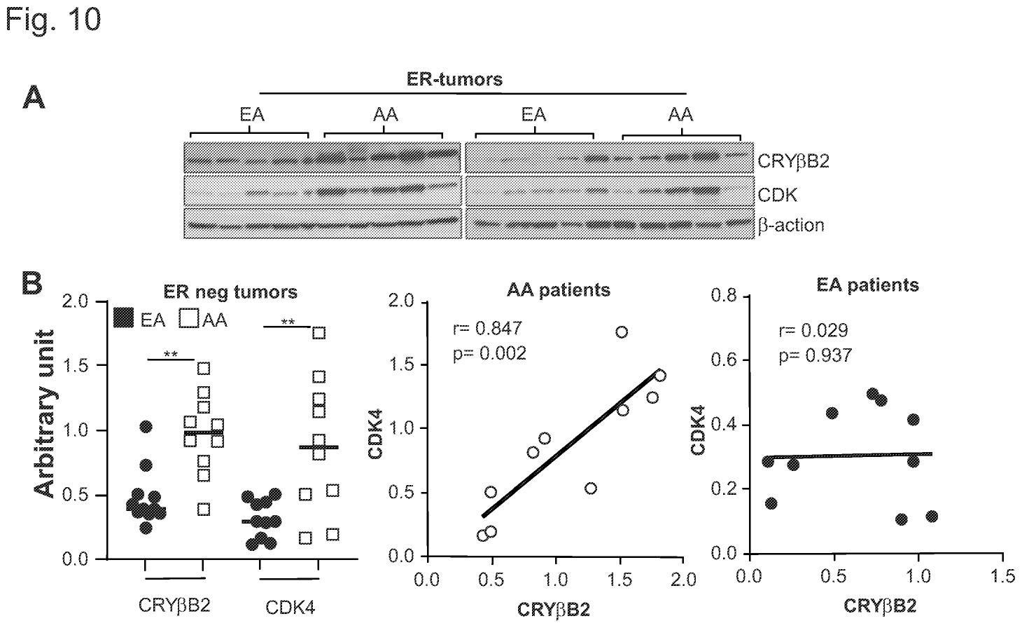

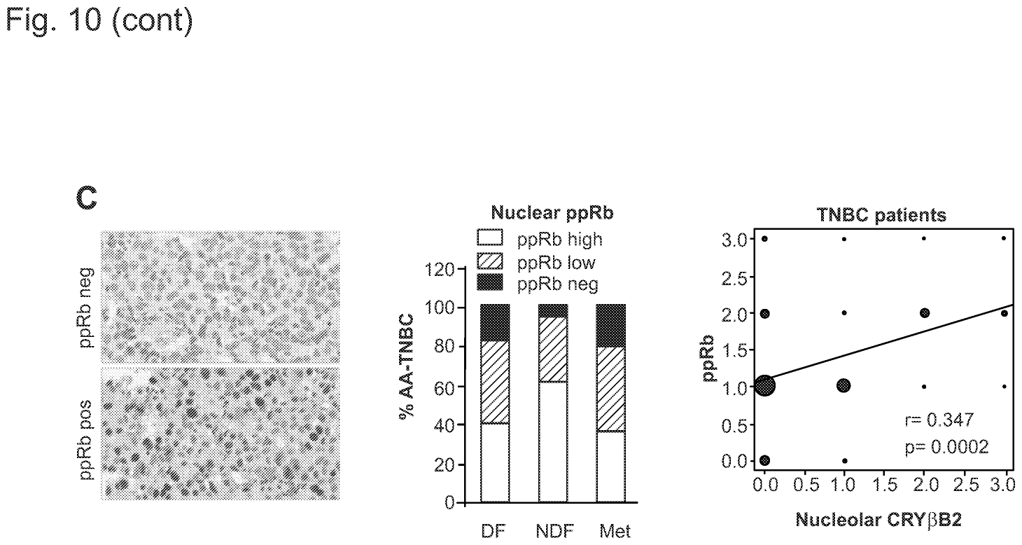

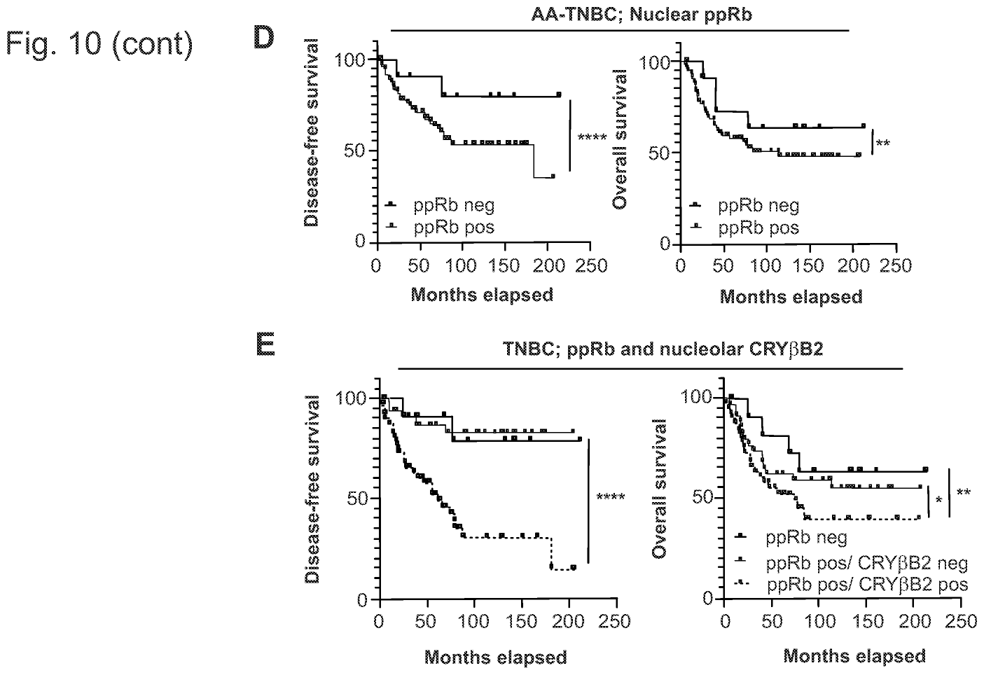

Higher expression and correlation of CRYβB2 and CDK4 proteins were observed exclusively in ER⁻ breast tumors of AA patients but not EA (FIG. 10A and FIG. 10B).

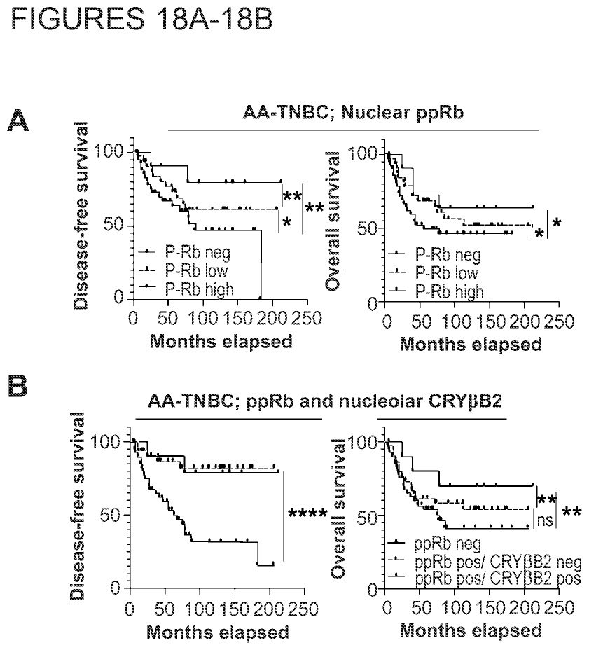

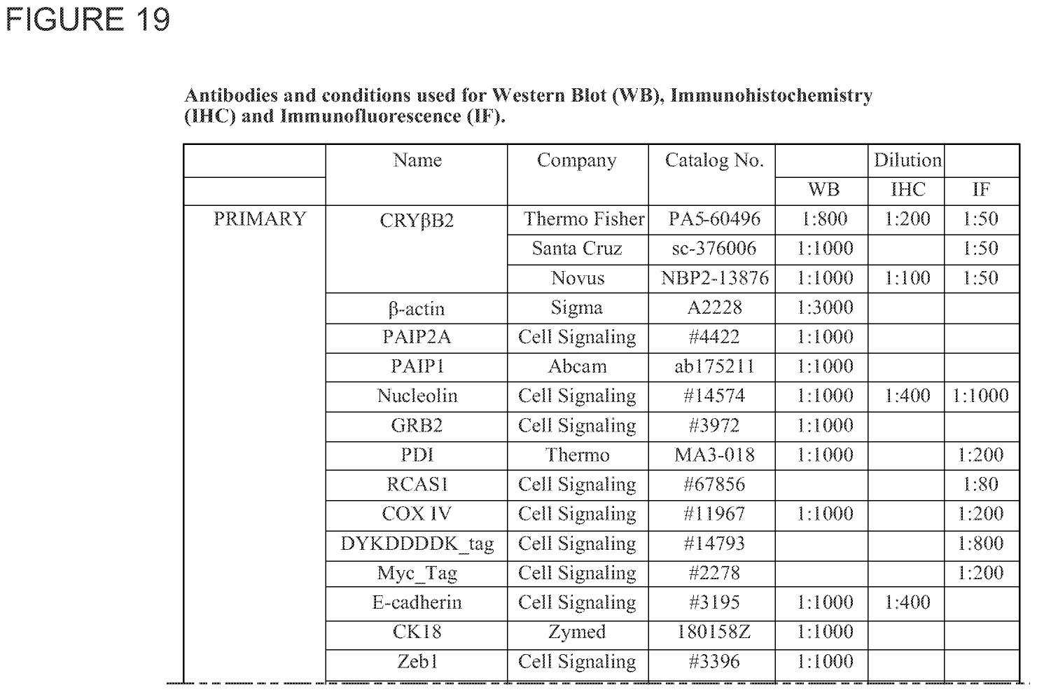

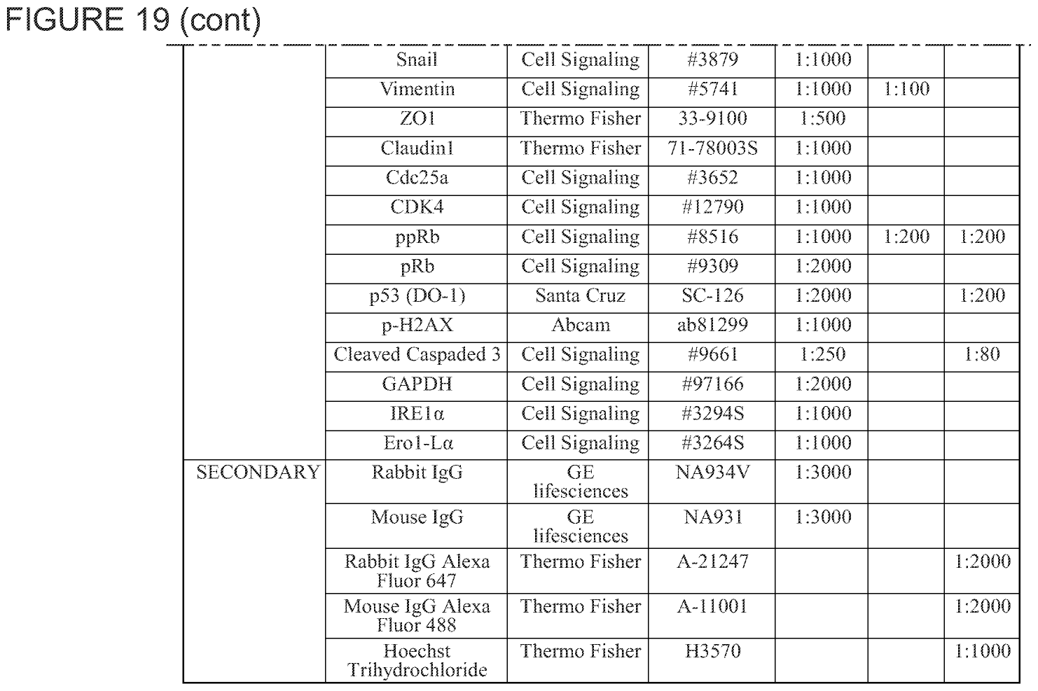

Since we observed that CRYβB2 activates the CDK4/pRb pathway in ER⁻ tumors of AA patients (FIG. 10A and FIG. 10B) we investigated the relationship between ppRb and CRYβB2 expression in tumors with the survival of the AA-TNBC patients. Our analysis showed that 85% (95% CI. 77%-92%) of TNBC from AA women were ppRb⁺ (n=102), with 46% having a ppRb high and 39% ppRb low expression (FIG. 10C). We detected ppRb expression in 94% (95% CI; 78%-99%) of tumors in AA TNBC patients that were never disease-free (n=33), with 61% having a ppRb high and 33% ppRb low expression (FIG. 10C). The ppRb protein expression correlated with nucleolar CRYβB2 expression in TNBC patients (FIG. 10C). The expression of ppRb in AA-TNBC associated with a significant decrease in disease-free (n=87, p=0.0002) and overall survival (n=87, p=0.0049) (FIG. 10D and FIG. 18A). Most importantly, patients with TNBC tumors expressing both CRYβB2 and ppRb (double positive) showed a more significant decrease in disease-free (p<0.0001) and overall survival (p=0.0456) than patients with ppRb⁺/CRYβB2⁻ tumors (FIG. 10E and FIG. 18B). Collectively, these data suggest that CDK4/pRb pathway is active and associates to poor prognosis in AA-TNBC patients. This finding is also consistent with previous data showing that an increased proliferation index of breast tumors is a poor prognosis marker for the disease.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. I. Menashe, W. F. Anderson, I. Jatoi, P. S. Rosenberg, Underlying causes of the black-white racial disparity in breast cancer mortality: a population-based analysis. Journal of the National Cancer Institute 101, 993-1000 (2009).
2. E. C. Dietze, C. Sistrunk, G. Miranda-Carboni, R. O'Regan, V. L. Seewaldt, Triple-negative breast cancer in African-American women: disparities versus biology. Nature reviews. Cancer 15, 248-254 (2015).
3. C. E. DeSantis, S. A. Fedewa, A. Goding Sauer, J. L. Kramer, R. A. Smith, A. Jemal, Breast cancer statistics, 2015: Convergence of incidence rates between black and white women. CA: a cancer journal for clinicians 66, 31-42 (2016).
4. C. K. Anders, L. A. Carey, Biology, metastatic patterns, and treatment of patients with triple-negative breast cancer. Clin Breast Cancer 9 Suppl 2, 573-81 (2009).
5. K. R. Bauer, M. Brown, R. D. Cress, C. A. Parise, V. Caggiano, Descriptive analysis of estrogen receptor (ER)-negative, progesterone receptor (PR)-negative, and HER2-negative invasive breast cancer, the so-called triple-negative phenotype: a population-based study from the California cancer Registry. Cancer 109, 1721-1728 (2007).
6. L. A. Carey, Through a glass darkly: advances in understanding breast cancer biology, 2000-2010. Clin Breast Cancer 10, 188-195 (2010).
7. L. A. Carey, C. M. Perou, C. A. Livasy, L. G. Dressler, D. Cowan, K. Conway, G. Karaca, M. A. Troester, C. K. Tse, S. Edmiston, S. L. Deming, J. Geradts, M. C. Cheang, T. O. Nielsen, P. G. Moorman, H. S. Earp, R. C. Millikan, Race, breast cancer subtypes, and survival in the Carolina Breast Cancer Study. JAMA 295, 2492-2502 (2006).
8. K. M. O'Brien, S. R. Cole, C. K. Tse, C. M. Perou, L. A. Carey, W. D. Foulkes, L. G. Dressler, J. Geradts, R. C. Millikan, Intrinsic breast tumor subtypes, race, and long-term survival in the Carolina Breast Cancer Study. Clinical cancer research: an official journal of the American Association for Cancer Research 16, 6100-6110 (2010).
9. D. N. Martin, B. J. Boersma, M. Yi, M. Reimers, T. M. Howe, H. G. Yfantis, Y. C. Tsai, E. H. Williams, D. H. Lee, R. M. Stephens, A. M. Weissman, S. Ambs, Differences in the tumor microenvironment between African-American and European-American breast cancer patients. PLoS One 4, e4531 (2009).
10. L. A. Field, B. Love, B. Deyarmin, J. A. Hooke, C. D. Shriver, R. E. Ellsworth, Identification of differentially expressed genes in breast tumors from African American compared with Caucasian women. Cancer 118, 1334-1344 (2012).
11. P. A. Stewart, J. Luks, M. D. Roycik, Q. X. Sang, J. Zhang, Differentially expressed transcripts and dysregulated signaling pathways and networks in African American breast cancer. PLoS One 8, e82460 (2013).
12. R. Lindner, C. Sullivan, O. Offor, K. Lezon-Geyda, K. Halligan, N. Fischbach, M. Shah, V. Bossuyt, V. Schulz, D. P. Tuck, L. N. Harris, Molecular phenotypes in triple negative breast cancer from African American patients suggest targets for therapy. PLoS One 8, e71915 (2013).
13. C. B. Ambrosone, A. C. Young, L. E. Sucheston, D. Wang, L. Yan, S. Liu, L. Tang, Q. Hu, J. L. Freudenheim, P. G. Shields, C. D. Morrison, K. Demissie, M. J. Higgins, Genome-wide methylation patterns provide insight into differences in breast tumor biology between American women of African and European ancestry. Oncotarget 5, 237-248 (2014).
14. T. A. Wallace, R. L. Prueitt, M. Yi, T. M. Howe, J. W. Gillespie, H. G. Yfantis, R. M. Stephens, N. E. Caporaso, C. A. Loffredo, S. Ambs, Tumor immunobiological differences in prostate cancer between African-American and European-American men. Cancer Res 68, 927-936 (2008).
15. B. Jovov, F. Araujo-Perez, C. S. Sigel, J. K. Stratford, A. N. McCoy, J. J. Yeh, T. Keku, Differential gene expression between African American and European American colorectal cancer patients. PLoS One 7, e30168 (2012).
16. D. Huo, H. Hu, S. K. Rhie, E. R. Gamazon, A. D. Cherniack, J. Liu, T. F. Yoshimatsu, J. J. Pitt, K. A. Hoadley, M. Troester, Y. Ru, T. Lichtenberg, L. A. Sturtz, C. S. Shelley, C. C. Benz, G. B. Mills, P. W. Laird, C. D. Shriver, C. M. Perou, O. I. Olopade, Comparison of Breast Cancer Molecular Features and Survival by African and European Ancestry in The Cancer Genome Atlas. JAMA oncology 3, 1654-1662 (2017).
17. M. A. Barrow, M. E. Martin, A. Coffey, P. L. Andrews, G. S. Jones, D. K. Reaves, J. S. Parker, M. A. Troester, J. M. Fleming, A functional role for the cancer disparity-linked genes, CRYbetaB2 and CRYbetaB2P1, in the promotion of breast cancer. Breast cancer research: BCR 21, 105 (2019).
18. J. V. Moyano, J. R. Evans, F. Chen, M. Lu, M. E. Werner, F. Yehiely, L. K. Diaz, D. Turbin, G. Karaca, E. Wiley, T. O. Nielsen, C. M. Perou, V. L. Cryns, AlphaB-crystallin is a novel oncoprotein that predicts poor clinical outcome in breast cancer. The Journal of clinical investigation 116, 261-270 (2006).
19. J. Graw, Genetics of crystallins: cataract and beyond. Experimental eye research 88, 173-189 (2009).
20. F. Anders, J. Teister, A. Liu, S. Funke, F. H. Grus, S. Thanos, H. D. von Pein, N. Pfeiffer, V. Prokosch, Intravitreal injection of beta-crystallin B2 improves retinal ganglion cell survival in an experimental animal model of glaucoma. PLoS One 12, e0175451 (2017).
21. T. Liedtke, J. C. Schwamborn, U. Schroer, S. Thanos, Elongation of axons during regeneration involves retinal crystallin beta b2 (crybb2). Molecular & cellular proteomics: MCP 6, 895-907 (2007).
22. Q. Gao, L. L. Sun, F. F. Xiang, L. Gao, Y. Jia, J. R. Zhang, H. B. Tao, J. J. Zhang, W. J. Li, Crybb2 deficiency impairs fertility in female mice. Biochemical and biophysical research communications 453, 37-42 (2014).
23. L. A. Sturtz, J. Melley, K. Mamula, C. D. Shriver, R. E. Ellsworth, Outcome disparities in African American women with triple negative breast cancer: a comparison of epidemiological and molecular factors between African American and Caucasian women with triple negative breast cancer. BMC cancer 14, 62 (2014).
24. P. J. Dawson, S. R. Wolman, L. Tait, G. H. Heppner, F. R. Miller, MCF10AT: a model for the evolution of cancer from proliferative breast disease. The American journal of pathology 148, 313-319 (1996).
25. F. R. Miller, S. J. Santner, L. Tait, P. J. Dawson, MCF10DCIS.com xenograft model of human comedo ductal carcinoma in situ. Journal of the National Cancer Institute 92, 1185-1186 (2000).
26. K. Asha, N. Sharma-Walia, Virus and tumor microenvironment induced ER stress and unfolded protein response: from complexity to therapeutics. Oncotarget 9, 31920-31936 (2018).
27. D. J. Paulucci, J. P. Sfakianos, A. J. Skanderup, K. Kan, C. K. Tsao, M. D. Galsky, A. A. Hakimi, K. K. Badani, Genomic differences between black and white patients implicate a distinct immune response to papillary renal cell carcinoma. Oncotarget 8, 5196-5205 (2017).
28. E. K. Schmidt, G. Clavarino, M. Ceppi, P. Pierre, SUnSET, a nonradioactive method to monitor protein synthesis. Nature methods 6, 275-277 (2009).
29. B. Bugler, M. Caizergues-Ferrer, G. Bouche, H. Bourbon, F. Amalric, Detection and localization of a class of proteins immunologically related to a 100-kDa nucleolar protein. European journal of biochemistry 128, 475-480 (1982).
30. Z. Chen, X. Xu, Roles of nucleolin. Focus on cancer and anti-cancer therapy. Saudi medical journal 37, 1312-1318 (2016).
31. A. Yang, G. Shi, C. Zhou, R. Lu, H. Li, L. Sun, Y. Jin, Nucleolin maintains embryonic stem cell self-renewal by suppression of p53 protein-dependent pathway. The Journal of biological chemistry 286, 43370-43382 (2011).
32. C. Mahotka, S. Bhatia, J. Kollet, E. Grinstein, Nucleolin promotes execution of the hematopoietic stem cell gene expression program. Leukemia 32, 1865-1868 (2018).
33. N. A. Fonseca, A. S. Rodrigues, P. Rodrigues-Santos, V. Alves, A. C. Gregorio, A. Valerio-Fernandes, L. C. Gomes-da-Silva, M. S. Rosa, V. Moura, J. Ramalho-Santos, S. Simoes, J. N. Moreira, Nucleolin overexpression in breast cancer cell sub-populations with different stem-like phenotype enables targeted intracellular delivery of synergistic drug combination. Biomaterials 69, 76-88 (2015).
34. E. Grinstein, Y. Shan, L. Karawajew, P. J. Snijders, C. J. Meijer, H. D. Royer, P. Wernet, Cell cycle-controlled interaction of nucleolin with the retinoblastoma protein and cancerous cell transformation. The Journal of biological chemistry 281, 22223-22235 (2006).
35. M. Takagi, M. J. Absalon, K. G. McLure, M. B. Kastan, Regulation of p53 translation and induction after DNA damage by ribosomal protein L26 and nucleolin. Cell 123, 49-63 (2005).
36. H. Konishi, M. Mohseni, A. Tamaki, J. P. Garay, S. Croessmann, S. Karnan, A. Ota, H. Y. Wong, Y. Konishi, B. Karakas, K. Tahir, A. M. Abukhdeir, J. P. Gustin, J. Cidado, G. M. Wang, D. Cosgrove, R. Cochran, D. Jelovac, M. J. Higgins, S. Arena, L. Hawkins, J. Lauring, A. L. Gross, C. M. Heaphy, Y. Hosokawa, E. Gabrielson, A. K. Meeker, K. Visvanathan, P. Argani, K. E. Bachman, B. H. Park, Mutation of a single allele of the cancer susceptibility gene BRCA1 leads to genomic instability in human breast epithelial cells. Proc Natl Acad Sci USA 108, 17773-17778 (2011).
37. S. Croessmann, H. Y. Wong, D. J. Zabransky, D. Chu, J. Mendonca, A. Sharma, M. Mohseni, D. M. Rosen, R. B. Scharpf, J. Cidado, R. L. Cochran, H. A. Parsons, W. B. Dalton, B. Erlanger, B. Button, K. Cravero, K. Kyker-Snowman, J. A. Beaver, S. Kachhap, P. J. Hurley, J. Lauring, B. H. Park, NDRG1 links p53 with proliferation-mediated centrosome homeostasis and genome stability. Proc Natl Acad Sci USA 112, 11583-11588 (2015).
38. M. B. Weiss, M. I. Vitolo, M. Mohseni, D. M. Rosen, S. R. Denmeade, B. H. Park, D. J. Weber, K. E. Bachman, Deletion of p53 in human mammary epithelial cells causes chromosomal instability and altered therapeutic response. Oncogene 29, 4715-4724 (2010).
39. R. S. Finn, J. Dering, D. Conklin, O. Kalous, D. J. Cohen, A. J. Desai, C. Ginther, M. Atefi, I. Chen, C. Fowst, G. Los, D. J. Slamon, PD 0332991, a selective cyclin D kinase 4/6 inhibitor, preferentially inhibits proliferation of luminal estrogen receptor-positive human breast cancer cell lines in vitro. Breast cancer research: BCR 11, R77 (2009).
40. D. Hanahan, R. A. Weinberg, The hallmarks of cancer. Cell 100, 57-70 (2000).
41. L. Li, D. B. Fan, Y. T. Zhao, Y. Li, D. Q. Kong, F. F. Cai, G. Y. Zheng, Two novel mutations identified in ADCC families impair crystallin protein distribution and induce apoptosis in human lens epithelial cells. Scientific reports 7, 17848 (2017).
42. L. Sisinni, M. Pietrafesa, S. Lepore, F. Maddalena, V. Condelli, F. Esposito, M. Landriscina, Endoplasmic Reticulum Stress and Unfolded Protein Response in Breast Cancer: The Balance between Apoptosis and Autophagy and Its Role in Drug Resistance. International journal of molecular sciences 20, (2019).
43. F. Alvandi, V. E. Kwitkowski, C. W. Ko, M. D. Rothmann, S. Ricci, H. Saber, D. Ghosh, J. Brown, E. Pfeiler, E. Chikhale, J. Grillo, J. Bullock, R. Kane, E. Kaminskas, A. T. Farrell, R. Pazdur, U.S. Food and Drug Administration approval summary: omacetaxine mepesuccinate as treatment for chronic myeloid leukemia. The oncologist 19, 94-99 (2014).
44. M. Yakhni, A. Briat, A. El Guerrab, L. Furtado, F. Kwiatkowski, E. Miot-Noirault, F. Cachin, F. Penault-Llorca, N. Radosevic-Robin, Homoharringtonine, an approved anti-leukemia drug, suppresses triple negative breast cancer growth through a rapid reduction of anti-apoptotic protein abundance. American journal of cancer research 9, 1043-1060 (2019).
45. Y. Yang, C. Yang, J. Zhang, C23 protein meditates bone morphogenetic protein-2-mediated EMT via up-regulation of Erk1/2 and Akt in gastric cancer. Medical oncology 32, 76 (2015).
46. P. J. Bates, E. M. Reyes-Reyes, M. T. Malik, E. M. Murphy, M. G. O'Toole, J. O. Trent, G-quadruplex oligonucleotide AS1411 as a cancer-targeting agent: Uses and mechanisms. Biochimica et biophysica acta. General subjects 1861, 1414-1428 (2017).
47. H. Xu, M. Di Antonio, S. McKinney, V. Mathew, B. Ho, N. J. O'Neil, N. D. Santos, J. Silvester, V. Wei, J. Garcia, F. Kabeer, D. Lai, P. Soriano, J. Banath, D. S. Chiu, D. Yap, D. D. Le, F. B. Ye, A. Zhang, K. Thu, J. Soong, S. C. Lin, A. H. Tsai, T. Osako, T. Algara, D. N. Saunders, J. Wong, J. Xian, M. B. Bally, J. D. Brenton, G. W. Brown, S. P. Shah, D. Cescon, T. W. Mak, C. Caldas, P. C. Stirling, P. Hieter, S. Balasubramanian, S. Aparicio, CX-5461 is a DNA G-quadruplex stabilizer with selective lethality in BRCA½ deficient tumours. Nature communications 8, 14432 (2017).
48. P. Carotenuto, A. Pecoraro, G. Palma, G. Russo, A. Russo, Therapeutic Approaches Targeting Nucleolus in Cancer. Cells 8, (2019).
49. M. Derenzini, D. Trere, A. Pession, L. Montanaro, V. Sirri, R. L. Ochs, Nucleolar function and size in cancer cells. The American journal of pathology 152, 1291-1297 (1998).
50. M. Derenzini, F. Nardi, F. Farabegoli, A. Ottinetti, F. Roncaroli, G. Bussolati, Distribution of silver-stained interphase nucleolar organizer regions as a parameter to distinguish neoplastic from nonneoplastic reactive cells in human effusions. Acta cytologica 33, 491-498 (1989).
51. I. Ugrinova, K. Monier, C. Ivaldi, M. Thiry, S. Storck, F. Mongelard, P. Bouvet, Inactivation of nucleolin leads to nucleolar disruption, cell cycle arrest and defects in centrosome duplication. BMC molecular biology 8, 66 (2007).
52. M. Cristofanilli, N. C. Turner, I. Bondarenko, J. Ro, S. A. Im, N. Masuda, M. Colleoni, A. DeMichele, S. Loi, S. Verma, H. Iwata, N. Harbeck, K. Zhang, K. P. Theall, Y. Jiang, C. H. Bartlett, M. Koehler, D. Slamon, Fulvestrant plus palbociclib versus fulvestrant plus placebo for treatment of hormone-receptor-positive, HER2-negative metastatic breast cancer that progressed on previous endocrine therapy (PALOMA-3): final analysis of the multicentre, double-blind, phase 3 randomised controlled trial. The Lancet. Oncology 17, 425-439 (2016).
53. A. Matutino, C. Amaro, S. Verma, CDK4/6 inhibitors in breast cancer: beyond hormone receptor-positive HER2-negative disease. Therapeutic advances in medical oncology 10, 1758835918818346 (2018).
54. G. W. Sledge, Jr., M. Toi, P. Neven, J. Sohn, K. Inoue, X. Pivot, O. Burdaeva, M. Okera, N. Masuda, P. A. Kaufman, H. Koh, E. M. Grischke, P. Conte, Y. Lu, S. Barriga, K. Hurt, M. Frenzel, S. Johnston, A. Llombart-Cussac, The Effect of Abemaciclib Plus Fulvestrant on Overall Survival in Hormone Receptor-Positive, ERBB2-Negative Breast Cancer That Progressed on Endocrine Therapy-MONARCH 2: A Randomized Clinical Trial. JAMA oncology, (2019).
55. S. F. Schoninger, S. W. Blain, The Ongoing Search for Biomarkers of CDK4/6 Inhibitor Responsiveness in Breast Cancer. Molecular cancer therapeutics 19, 3-12 (2020).
56. M. Gama-Carvalho, M. Carmo-Fonseca, The rules and roles of nucleocytoplasmic shuttling proteins. FEBS letters 498, 157-163 (2001).
57. N. Cancer Genome Atlas, Comprehensive molecular portraits of human breast tumours. Nature 490, 61-70 (2012).
58. M. Shipitsin, L. L. Campbell, P. Argani, S. Weremowicz, N. Bloushtain-Qimron, J. Yao, T. Nikolskaya, T. Serebryiskaya, R. Beroukhim, M. Hu, M. K. Halushka, S. Sukumar, L. M. Parker, K. S. Anderson, L. N. Harris, J. E. Garber, A. L. Richardson, S. J. Schnitt, Y. Nikolsky, R. S. Gelman, K. Polyak, Molecular definition of breast tumor heterogeneity. Cancer Cell 11, 259-273 (2007).
59. V. F. Merino, N. Nguyen, K. Jin, H. Sadik, S. Cho, P. Korangath, L. Han, Y. M. N. Foster, X. C. Zhou, Z. Zhang, R. M. Connolly, V. Steams, S. Z. Ali, C. Adams, Q. Chen, D. Pan, D. L. Huso, P. Ordentlich, A. Brodie, S. Sukumar, Combined Treatment with Epigenetic, Differentiating, and Chemotherapeutic Agents Cooperatively Targets Tumor-Initiating Cells in Triple-Negative Breast Cancer. Cancer Res 76, 2013-2024 (2016).
60. Y. Hu, G. K. Smyth, ELDA: extreme limiting dilution analysis for comparing depleted and enriched populations in stem cell and other assays. Journal of immunological methods 347, 70-78 (2009).
61. M. G. Pomper, H. Hammond, X. Yu, Z. Ye, C. A. Foss, D. D. Lin, J. J. Fox, L. Cheng, Serial imaging of human embryonic stem-cell engraftment and teratoma formation in live mouse models. Cell research 19, 370-379 (2009).
62. C. A. Foss, L. Kulik, A. A. Ordonez, S. K. Jain, V. Michael Holers, J. M. Thurman, M. G. Pomper, SPECT/CT Imaging of Mycobacterium tuberculosis Infection with [(125)I]anti-C3d mAb. Molecular imaging and biology 21, 473-481 (2019).
63. S. Hanzelmann, R. Castelo, J. Guinney, GSVA: gene set variation analysis for microarray and RNA-seq data. BMC bioinformatics 14, 7 (2013).
64. K. J. Yoon, G. Song, X. Qian, J. Pan, D. Xu, H. S. Rho, N. S. Kim, C. Habela, L. Zheng, F. Jacob, F. Zhang, E. M. Lee, W. K. Huang, F. R. Ringeling, C. Vissers, C. Li, L. Yuan, K. Kang, S. Kim, J. Yeo, Y. Cheng, S. Liu, Z. Wen, C. F. Qin, Q. Wu, K. M. Christian, H. Tang, P. Jin, Z. Xu, J. Qian, H. Zhu, H. Song, G. L. Ming, Zika-Virus-Encoded NS2A Disrupts Mammalian Cortical Neurogenesis by Degrading Adherens Junction Proteins. Cell stem cell 21, 349-358 e346 (2017).
1. I. Menashe, W. F. Anderson, I. Jatoi, P. S. Rosenberg, Underlying causes of the black-white racial disparity in breast cancer mortality: a population-based analysis. Journal of the National Cancer Institute 101, 993-1000 (2009).
2. E. C. Dietze, C. Sistrunk, G. Miranda-Carboni, R. O'Regan, V. L. Seewaldt, Triple-negative breast cancer in African-American women: disparities versus biology. Nature reviews. Cancer 15, 248-254 (2015).
3. C. E. DeSantis, S. A. Fedewa, A. Goding Sauer, J. L. Kramer, R. A. Smith, A. Jemal, Breast cancer statistics, 2015: Convergence of incidence rates between black and white women. CA: a cancer journal for clinicians 66, 31-42 (2016).
4. K. R. Bauer, M. Brown, R. D. Cress, C. A. Parise, V. Caggiano, Descriptive analysis of estrogen receptor (ER)-negative, progesterone receptor (PR)-negative, and HER2-negative invasive breast cancer, the so-called triple-negative phenotype: a population-based study from the California cancer Registry. Cancer 109, 1721-1728 (2007).
5. L. A. Carey, Through a glass darkly: advances in understanding breast cancer biology, 2000-2010. Clin Breast Cancer 10, 188-195 (2010).
6. L. A. Carey, C. M. Perou, C. A. Livasy, L. G. Dressler, D. Cowan, K. Conway, G. Karaca, M. A. Troester, C. K. Tse, S. Edmiston, S. L. Deming, J. Geradts, M. C. Cheang, T. O. Nielsen, P. G. Moorman, H. S. Earp, R. C. Millikan, Race, breast cancer subtypes, and survival in the Carolina Breast Cancer Study. JAMA 295, 2492-2502 (2006).
7. D. N. Martin, B. J. Boersma, M. Yi, M. Reimers, T. M. Howe, H. G. Yfantis, Y. C. Tsai, E. H. Williams, D. H. Lee, R. M. Stephens, A. M. Weissman, S. Ambs, Differences in the tumor microenvironment between African-American and European-American breast cancer patients. PLoS One 4, e4531 (2009).
8. L. A. Field, B. Love, B. Deyarmin, J. A. Hooke, C. D. Shriver, R. E. Ellsworth, Identification of differentially expressed genes in breast tumors from African American compared with Caucasian women. Cancer 118, 1334-1344 (2012).
9. P. A. Stewart, J. Luks, M. D. Roycik, Q. X. Sang, J. Zhang, Differentially expressed transcripts and dysregulated signaling pathways and networks in African American breast cancer. PLoS One 8, e82460 (2013).
10. R. Lindner, C. Sullivan, O. Offor, K. Lezon-Geyda, K. Halligan, N. Fischbach, M. Shah, V. Bossuyt, V. Schulz, D. P. Tuck, L. N. Harris, Molecular phenotypes in triple negative breast cancer from African American patients suggest targets for therapy. PLoS One 8, e71915 (2013).
11. C. B. Ambrosone, A. C. Young, L. E. Sucheston, D. Wang, L. Yan, S. Liu, L. Tang, Q. Hu, J. L. Freudenheim, P. G. Shields, C. D. Morrison, K. Demissie, M. J. Higgins, Genome-wide methylation patterns provide insight into differences in breast tumor biology between American women of African and European ancestry. Oncotarget 5, 237-248 (2014).
12. T. A. Wallace, R. L. Prueitt, M. Yi, T. M. Howe, J. W. Gillespie, H. G. Yfantis, R. M. Stephens, N. E. Caporaso, C. A. Loffredo, S. Ambs, Tumor immunobiological differences in prostate cancer between African-American and European-American men. Cancer research 68, 927-936 (2008).
13. B. Jovov, F. Araujo-Perez, C. S. Sigel, J. K. Stratford, A. N. McCoy, J. J. Yeh, T. Keku, Differential gene expression between African American and European American colorectal cancer patients. PLoS One 7, e30168 (2012).
14. D. Huo, H. Hu, S. K. Rhie, E. R. Gamazon, A. D. Cherniack, J. Liu, T. F. Yoshimatsu, J. J. Pitt, K. A. Hoadley, M. Troester, Y. Ru, T. Lichtenberg, L. A. Sturtz, C. S. Shelley, C. C. Benz, G. B. Mills, P. W. Laird, C. D. Shriver, C. M. Perou, O. I. Olopade, Comparison of Breast Cancer Molecular Features and Survival by African and European Ancestry in The Cancer Genome Atlas. JAMA oncology 3, 1654-1662 (2017).
15. M. A. Barrow, M. E. Martin, A. Coffey, P. L. Andrews, G. S. Jones, D. K. Reaves, J. S. Parker, M. A. Troester, J. M. Fleming, A functional role for the cancer disparity-linked genes, CRYbetaB2 and CRYbetaB2P1, in the promotion of breast cancer. Breast cancer research: BCR 21, 105 (2019).
16. J. V. Moyano, J. R. Evans, F. Chen, M. Lu, M. E. Werner, F. Yehiely, L. K. Diaz, D. Turbin, G. Karaca, E. Wiley, T. O. Nielsen, C. M. Perou, V. L. Cryns, AlphaB-crystallin is a novel oncoprotein that predicts poor clinical outcome in breast cancer. The Journal of clinical investigation 116, 261-270 (2006).
17. J. Graw, Genetics of crystallins: cataract and beyond. Experimental eye research 88, 173-189 (2009).
18. F. Anders, J. Teister, A. Liu, S. Funke, F. H. Grus, S. Thanos, H. D. von Pein, N. Pfeiffer, V. Prokosch, Intravitreal injection of beta-crystallin B2 improves retinal ganglion cell survival in an experimental animal model of glaucoma. PLoS One 12, e0175451 (2017).
19. T. Liedtke, J. C. Schwamborn, U. Schroer, S. Thanos, Elongation of axons during regeneration involves retinal crystallin beta b2 (crybb2). Molecular & cellular proteomics: MCP 6, 895-907 (2007).
20. Q. Gao, L. L. Sun, F. F. Xiang, L. Gao, Y. Jia, J. R. Zhang, H. B. Tao, J. J. Zhang, W. J. Li, Crybb2 deficiency impairs fertility in female mice. Biochemical and biophysical research communications 453, 37-42 (2014).
21. R. S. Finn, J. Dering, D. Conklin, O. Kalous, D. J. Cohen, A. J. Desai, C. Ginther, M. Atefi, I. Chen, C. Fowst, G. Los, D. J. Slamon, PD 0332991, a selective cyclin D kinase 4/6 inhibitor, preferentially inhibits proliferation of luminal estrogen receptor-positive human breast cancer cell lines in vitro. Breast cancer research: BCR 11, R77 (2009).
22. A. C. Gregorio, M. Lacerda, P. Figueiredo, S. Simoes, S. Dias, J. N. Moreira, Meeting the needs of breast cancer: A nucleolin's perspective. Critical reviews in oncology hematology 125, 89-101 (2018).
23. M. Shipitsin, L. L. Campbell, P. Argani, S. Weremowicz, N. Bloushtain-Qimron, J. Yao, T. Nikolskaya, T. Serebryiskaya, R. Beroukhim, M. Hu, M. K. Halushka, S. Sukumar, L. M. Parker, K. S. Anderson, L. N. Harris, J. E. Garber, A. L. Richardson, S. J. Schnitt, Y. Nikolsky, R. S. Gelman, K. Polyak, Molecular definition of breast tumor heterogeneity. Cancer Cell 11, 259-273 (2007).
24. V. F. Merino, N. Nguyen, K. Jin, H. Sadik, S. Cho, P. Korangath, L. Han, Y. M. N. Foster, X. C. Zhou, Z. Zhang, R. M. Connolly, V. Steams, S. Z. Ali, C. Adams, Q. Chen, D. Pan, D. L. Huso, P. Ordentlich, A. Brodie, S. Sukumar, Combined Treatment with Epigenetic, Differentiating, and Chemotherapeutic Agents Cooperatively Targets Tumor-Initiating Cells in Triple-Negative Breast Cancer. Cancer research 76, 2013-2024 (2016).
25. Y. Hu, G. K. Smyth, ELDA: extreme limiting dilution analysis for comparing depleted and enriched populations in stem cell and other assays. Journal of immunological methods 347, 70-78 (2009).
26. M. G. Pomper, H. Hammond, X. Yu, Z. Ye, C. A. Foss, D. D. Lin, J. J. Fox, L. Cheng, Serial imaging of human embryonic stem-cell engraftment and teratoma formation in live mouse models. Cell research 19, 370-379 (2009).
27. C. A. Foss, L. Kulik, A. A. Ordonez, S. K. Jain, V. Michael Holers, J. M. Thurman, M. G. Pomper, SPECT/CT Imaging of Mycobacterium tuberculosis Infection with [(125)I]anti-C3d mAb. Molecular imaging and biology 21, 473-481 (2019).
28. S. Hanzelmann, R. Castelo, J. Guinney, GSVA: gene set variation analysis for microarray and RNA-seq data. BMC bioinformatics 14, 7 (2013).
29. K. J. Yoon, G. Song, X. Qian, J. Pan, D. Xu, H. S. Rho, N. S. Kim, C. Habela, L. Zheng, F. Jacob, F. Zhang, E. M. Lee, W. K. Huang, F. R. Ringeling, C. Vissers, C. Li, L. Yuan, K. Kang, S. Kim, J. Yeo, Y. Cheng, S. Liu, Z. Wen, C. F. Qin, Q. Wu, K. M. Christian, H. Tang, P. Jin, Z. Xu, J. Qian, H. Zhu, H. Song, G. L. Ming, Zika-Virus-Encoded NS2A Disrupts Mammalian Cortical Neurogenesis by Degrading Adherens Junction Proteins. Cell stem cell 21, 349-358 e346 (2017).
30. J. O. Liu, Z. Guo, Z. Cheng, J. Wang, W. Liu, H. Peng, Y. Wang, S. Rao, R. Li, X. Ying, P. Korangath, M. Liberti, Y. Li, Y. Xie, S. Hong, C. Schiene-Fischer, G. Fischer, J. Locasale, S. Sukumar, H. Zhu, Discovery of a potent GLUT inhibitor using rapafucin 3D microarrays. Angewandte Chemie, (2019).
31. P. Korangath, W. W. Teo, H. Sadik, L. Han, N. Mori, C. M. Huijts, F. Wildes, S. Bharti, Z. Zhang, C. A. Santa-Maria, H. Tsai, C. V. Dang, V. Steams, Z. M. Bhujwalla, S. Sukumar, Targeting Glutamine Metabolism in Breast Cancer with Aminooxyacetate. Clin Cancer Res, (2015).
32. G. Dontu, W. M. Abdallah, J. M. Foley, K. W. Jackson, M. F. Clarke, M. J. Kawamura, M. S. Wicha, In vitro propagation and transcriptional profiling of human mammary stem/progenitor cells. Genes Dev 17, 1253-1270 (2003).
33. L. A. Sturtz, J. Melley, K. Mamula, C. D. Shriver, R. E. Ellsworth, Outcome disparities in African American women with triple negative breast cancer: a comparison of epidemiological and molecular factors between African American and Caucasian women with triple negative breast cancer. BMC cancer 14, 62 (2014).
34. P. J. Dawson, S. R. Wolman, L. Tait, G. H. Heppner, F. R. Miller, MCF10AT: a model for the evolution of cancer from proliferative breast disease. The American journal of pathology 148, 313-319 (1996).
35. F. R. Miller, S. J. Santner, L. Tait, P. J. Dawson, MCF10DCIS.com xenograft model of human comedo ductal carcinoma in situ. Journal of the National Cancer Institute 92, 1185-1186 (2000).
36. K. Asha, N. Sharma-Walia, Virus and tumor microenvironment induced ER stress and unfolded protein response: from complexity to therapeutics. Oncotarget 9, 31920-31936 (2018).
37. D. J. Paulucci, J. P. Sfakianos, A. J. Skanderup, K. Kan, C. K. Tsao, M. D. Galsky, A. A. Hakimi, K. K. Badani, Genomic differences between black and white patients implicate a distinct immune response to papillary renal cell carcinoma. Oncotarget 8, 5196-5205 (2017).
38. E. K. Schmidt, G. Clavarino, M. Ceppi, P. Pierre, SUnSET, a nonradioactive method to monitor protein synthesis. Nature methods 6, 275-277 (2009).
39. K. Khaleghpour, Y. V. Svitkin, A. W. Craig, C. T. DeMaria, R. C. Deo, S. K. Burley, N. Sonenberg, Translational repression by a novel partner of human poly(A) binding protein, Paip2. Molecular cell 7, 205-216 (2001).
40. J. Shorter, M. B. Beard, J. Seemann, A. B. Dirac-Svejstrup, G. Warren, Sequential tethering of Golgins and catalysis of SNAREpin assembly by the vesicle-tethering protein p115. The Journal of cell biology 157, 45-62 (2002).
41. A. W. Craig, A. Haghighat, A. T. Yu, N. Sonenberg, Interaction of polyadenylate-binding protein with the eIF4G homologue PAIP enhances translation. Nature 392, 520-523 (1998).
42. J. K. Herzig, L. Bullinger, A. Tasdogan, P. Zimmermann, M. Schlegel, V. Teleanu, D. Weber, F. G. Rucker, P. Paschka, A. Dolnik, E. Schneider, F. Kuchenbauer, F. H. Heidel, C. Buske, H. Dohner, K. Dohner, V. I. Gaidzik, Protein phosphatase 4 regulatory subunit 2 (PPP4R2) is recurrently deleted in acute myeloid leukemia and required for efficient DNA double strand break repair. Oncotarget 8, 95038-95053 (2017).
43. P. S. Page-McCaw, K. Amonlirdviman, P. A. Sharp, PUF60: a novel U2AF65-related splicing activity. Rna 5, 1548-1560 (1999).
44. Y. Shinoda, K. Fujita, S. Saito, H. Matsui, Y. Kanto, Y. Nagaura, K. Fukunaga, S. Tamura, T. Kobayashi, Acyl-CoA binding domain containing 3 (ACBD3) recruits the protein phosphatase PPM1L to ER-Golgi membrane contact sites. FEBS letters 586, 3024-3029 (2012).
45. Y. Huang, L. Yang, Y. Y. Pei, J. Wang, H. Wu, J. Yuan, L. Wang, Overexpressed ACBD3 has prognostic value in human breast cancer and promotes the self-renewal potential of breast cancer cells by activating the Wnt/beta-catenin signaling pathway. Experimental cell research 363, 39-47 (2018).
46. G. M. Bokoch, Caspase-mediated activation of PAK2 during apoptosis: proteolytic kinase activation as a general mechanism of apoptotic signal transduction? Cell death and differentiation 5, 637-645 (1998).
47. K. Bhardwaj, P. Liu, J. L. Leibowitz, C. C. Kao, The coronavirus endoribonuclease Nsp15 interacts with retinoblastoma tumor suppressor protein. Journal of virology 86, 4294-4304 (2012).
48. W. Jia, Z. Yao, J. Zhao, Q. Guan, L. Gao, New perspectives of physiological and pathological functions of nucleolin (NCL). Life sciences 186, 1-10 (2017).
49. J. Fu, B. Luo, W. W. Guo, Q. M. Zhang, L. Shi, Q. P. Hu, F. Chen, S. W. Xiao, X. X. Xie, Down-regulation of cancer/testis antigen OY-TES-1 attenuates malignant behaviors of hepatocellular carcinoma cells in vitro. International journal of clinical and experimental pathology 8, 7786-7797 (2015).
50. M. Ijaz, F. Wang, M. Shahbaz, W. Jiang, A. H. Fathy, E. U. Nesa, The Role of Grb2 in Cancer and Peptides as Grb2 Antagonists. Protein and peptide letters 24, 1084-1095 (2018).
51. X. Yang, K. Wu, S. Li, L. Hu, J. Han, D. Zhu, X. Tian, W. Liu, Z. Tian, L. Zhong, M. Yan, C. Zhang, Z. Zhang, MFAP5 and TNNC1: Potential markers for predicting occult cervical lymphatic metastasis and prognosis in early stage tongue cancer. Oncotarget 8, 2525-2535 (2017).
52. L. Alfano, A. Caporaso, A. Altieri, M. Dell'Aquila, C. Landi, L. Bini, F. Pentimalli, A. Giordano, Depletion of the RNA binding protein HNRNPD impairs homologous recombination by inhibiting DNA-end resection and inducing R-loop accumulation. Nucleic acids research 47, 4068-4085 (2019).
53. A. Sullivan, C. R. Uff, C. M. Isacke, R. F. Thorne, PACE-1, a novel protein that interacts with the C-terminal domain of ezrin. Experimental cell research 284, 224-238 (2003).
54. M. Sharma, M. R. Blackman, M. C. Sharma, Antibody-directed neutralization of annexin II (ANX II) inhibits neoangiogenesis and human breast tumor growth in a xenograft model. Experimental and molecular pathology 92, 175-184 (2012).
55. Y. J. Zhuo, Z. Z. Liu, S. Wan, Z. D. Cai, J. J. Xie, Z. D. Cai, S. D. Song, Y. P. Wan, W. Hua, W. Zhong, C. L. Wu, Enhanced expression of SRPK2 contributes to aggressive progression and metastasis in prostate cancer. Biomedicine & pharmacotherapy=Biomedecine & pharmacotherapie 102, 531-538 (2018).
56. Y. Ning, N. Hui, B. Qing, Y. Zhuo, W. Sun, Y. Du, S. Liu, K. Liu, J. Zhou, ZCCHC10 suppresses lung cancer progression and cisplatin resistance by attenuating MDM2-mediated p53 ubiquitination and degradation. Cell death & disease 10, 414 (2019).
57. M. Wang, X. Li, J. Zhang, Q. Yang, W. Chen, W. Jin, Y. R. Huang, R. Yang, W. Q. Gao, AHNAK2 is a Novel Prognostic Marker and Oncogenic Protein for Clear Cell Renal Cell Carcinoma. Theranostics 7, 1100-1113 (2017).
58. E. H. F. Jandrey, R. P. Moura, L. N. S. Andrade, C. L. Machado, L. F. Campesato, K. R. M. Leite, L. T. Inoue, P. F. Asprino, A. P. M. da Silva, A. de Barros, A. Carvalho, V. C. de Lima, D. M. Carraro, H. P. Brentani, I. W. da Cunha, F. A. Soares, R. B. Parmigiani, R. Chammas, A. A. Camargo, E. T. Costa, NDRG4 promoter hypermethylation is a mechanistic biomarker associated with metastatic progression in breast cancer patients. NPJ breast cancer 5, 11 (2019).
59. J. Shi, J. Ye, H. Fei, S. H. Jiang, Z. Y. Wu, Y. P. Chen, L. W. Zhang, X. M. Yang, YWHAZ promotes ovarian cancer metastasis by modulating glycolysis. Oncology reports 41, 1101-1112 (2019).
60. Z. Li, H. R. Park, Z. Shi, Z. Li, C. D. Pham, Y. Du, F. R. Khuri, Y. Zhang, Q. Han, H. Fu, Pro-oncogenic function of HIP-55/Drebrin-like (DBNL) through Ser269/Thr291-phospho-sensor motifs. Oncotarget 5, 3197-3209 (2014).
61. I. Keklikoglou, C. Cianciaruso, E. Guc, M. L. Squadrito, L. M. Spring, S. Tazzyman, L. Lambein, A. Poissonnier, G. B. Ferraro, C. Baer, A. Cassara, A. Guichard, M. L. Iruela-Arispe, C. E. Lewis, L. M. Coussens, A. Bardia, R. K. Jain, J. W. Pollard, M. De Palma, Chemotherapy elicits pro-metastatic extracellular vesicles in breast cancer models. Nature cell biology 21, 190-202 (2019).
62. C. M. Torres, A. Biran, M. J. Burney, H. Patel, T. Henser-Brownhill, A. S. Cohen, Y. Li, R. Ben-Hamo, E. Nye, B. Spencer-Dene, P. Chakravarty, S. Efroni, N. Matthews, T. Misteli, E. Meshorer, P. Scaffidi, The linker histone H1.0 generates epigenetic and functional intratumor heterogeneity. Science 353, (2016).
63. L. A. Moraes, S. Kar, S. L. Foo, T. Gu, Y. Q. Toh, P. B. Ampomah, K. Sachaphibulkij, G. Yap, O. Zharkova, H. M. Lukman, A. M. Fairhurst, A. P. Kumar, L. H. K. Lim, Annexin-A1 enhances breast cancer growth and migration by promoting alternative macrophage polarization in the tumour microenvironment. Scientific reports 7, 17925 (2017).
64. B. Peng, C. Guo, H. Guan, S. Liu, M. Z. Sun, Annexin A5 as a potential marker in tumors. Clinica chimica acta; international journal of clinical chemistry 427, 42-48 (2014).
65. Y. Jung, J. H. McCarty, Band 4.1 proteins regulate integrin-dependent cell spreading. Biochemical and biophysical research communications 426, 578-584 (2012).
66. S. T. Lim, D. Mikolon, D. G. Stupack, D. D. Schlaepfer, FERM control of FAK function: implications for cancer therapy. Cell cycle 7, 2306-2314 (2008).
67. I. Adam, D. L. Dewi, J. Mooiweer, A. Sadik, S. R. Mohapatra, B. Berdel, M. Keil, J. K. Sonner, K. Thedieck, A. J. Rose, M. Platten, I. Heiland, S. Trump, C. A. Opitz, Upregulation of tryptophanyl-tRNA synthethase adapts human cancer cells to nutritional stress caused by tryptophan degradation. Oncoimmunology 7, e1486353 (2018).
68. Y. Sun, H. Zhang, M. Kazemian, J. M. Troy, C. Seward, X. Lu, L. Stubbs, ZSCAN5B and primate-specific paralogs bind RNA polymerase III genes and extra-TFIIIC (ETC) sites to modulate mitotic progression. Oncotarget 7, 72571-72592 (2016).
69. B. Bugler, M. Caizergues-Ferrer, G. Bouche, H. Bourbon, F. Amalric, Detection and localization of a class of proteins immunologically related to a 100-kDa nucleolar protein. European journal of biochemistry 128, 475-480 (1982).
70. H. Konishi, M. Mohseni, A. Tamaki, J. P. Garay, S. Croessmann, S. Kaman, A. Ota, H. Y. Wong, Y. Konishi, B. Karakas, K. Tahir, A. M. Abukhdeir, J. P. Gustin, J. Cidado, G. M. Wang, D. Cosgrove, R. Cochran, D. Jelovac, M. J. Higgins, S. Arena, L. Hawkins, J. Lauring, A. L. Gross, C. M. Heaphy, Y. Hosokawa, E. Gabrielson, A. K. Meeker, K. Visvanathan, P. Argani, K. E. Bachman, B. H. Park, Mutation of a single allele of the cancer susceptibility gene BRCA1 leads to genomic instability in human breast epithelial cells. Proceedings of the National Academy of Sciences of the United States of America 108, 17773-17778 (2011).
71. Z. Tu, K. M. Aird, R. Zhang, RAS, cellular senescence and transformation: the BRCA1 DNA repair pathway at the crossroads. Small GTPases 3, 163-167 (2012).
72. S. Zeng, W. H. Shen, L. Liu, Senescence and Cancer. Cancer translational medicine 4, 70-74 (2018).
73. Z. Chen, X. Xu, Roles of nucleolin. Focus on cancer and anti-cancer therapy. Saudi medical journal 37, 1312-1318 (2016).
74. A. Yang, G. Shi, C. Zhou, R. Lu, H. Li, L. Sun, Y. Jin, Nucleolin maintains embryonic stem cell self-renewal by suppression of p53 protein-dependent pathway. The Journal of biological chemistry 286, 43370-43382 (2011).
75. C. Mahotka, S. Bhatia, J. Kollet, E. Grinstein, Nucleolin promotes execution of the hematopoietic stem cell gene expression program. Leukemia 32, 1865-1868 (2018).
76. N. A. Fonseca, A. S. Rodrigues, P. Rodrigues-Santos, V. Alves, A. C. Gregorio, A. Valerio-Fernandes, L. C. Gomes-da-Silva, M. S. Rosa, V. Moura, J. Ramalho-Santos, S. Simoes, J. N. Moreira, Nucleolin overexpression in breast cancer cell sub-populations with different stem-like phenotype enables targeted intracellular delivery of synergistic drug combination. Biomaterials 69, 76-88 (2015).
77. E. Grinstein, Y. Shan, L. Karawajew, P. J. Snijders, C. J. Meijer, H. D. Royer, P. Wernet, Cell cycle-controlled interaction of nucleolin with the retinoblastoma protein and cancerous cell transformation. The Journal of biological chemistry 281, 22223-22235 (2006).
78. M. Takagi, M. J. Absalon, K. G. McLure, M. B. Kastan, Regulation of p53 translation and induction after DNA damage by ribosomal protein L26 and nucleolin. Cell 123, 49-63 (2005).
79. S. Croessmann, H. Y. Wong, D. J. Zabransky, D. Chu, J. Mendonca, A. Sharma, M. Mohseni, D. M. Rosen, R. B. Scharpf, J. Cidado, R. L. Cochran, H. A. Parsons, W. B. Dalton, B. Erlanger, B. Button, K. Cravero, K. Kyker-Snowman, J. A. Beaver, S. Kachhap, P. J. Hurley, J. Lauring, B. H. Park, NDRG1 links p53 with proliferation-mediated centrosome homeostasis and genome stability. Proceedings of the National Academy of Sciences of the United States of America 112, 11583-11588 (2015).
80. M. B. Weiss, M. I. Vitolo, M. Mohseni, D. M. Rosen, S. R. Denmeade, B. H. Park, D. J. Weber, K. E. Bachman, Deletion of p53 in human mammary epithelial cells causes chromosomal instability and altered therapeutic response. Oncogene 29, 4715-4724 (2010).

1. A method for identifying a female subject as having a breast tumor which is responsive to a CDK4 inhibitor, an anti-nucleolin agent or a ribosomal RNA synthesis inhibitor, the method comprising:
a) testing a breast tumor tissue sample from the tumor of the subject for expression of CRYβB2 protein in the cells of the sample;
b) comparing the level of expression in the sample of the subject with the level of expression in a reference breast tissue sample; and
c) identifying the subject as having a breast tumor that is CRYβB2 positive and may likely respond to a CDK4 inhibitor an anti-nucleolin agent or a ribosomal RNA synthesis inhibitor when the level of expression of CRYβB2 is greater than the level of expression of CRYβB2 in the reference sample.

2-3. (canceled)

4. The method of claim 1, wherein the breast cancer is estrogen receptor positive.

5. The method of claim 1, wherein the breast cancer is triple negative.

6. The method of claim 1, wherein the female subject is African American.

7. The method of claim 1, wherein the breast tumor responds to a CDK4 inhibitor that is selected from the group consisting of palbociclib, ribociclib, and abemaciclib.

8. The method of claim 1, wherein the breast tumor responds to an anti-nucleolin agent that is an aptamer that binds nucleolin RNA

9. The method of claim 8, wherein the aptamer is AS 1411.

10. The method of claim 1, wherein the breast tumor responds to an inhibitor of ribosome RNA synthesis that is CX-5461 or BMH-21 and analogs thereof.

11. A method for treating a female subject having a breast tumor which is responsive to a CDK4 inhibitor, an anti-nucleolin agent or a ribosomal RNA synthesis inhibitor comprising:
a) testing a breast tumor tissue sample from the tumor of the subject for expression of CRYβB2 protein in the cells of the sample;
b) comparing the level of expression in the sample of the subject with the level of expression in a reference breast tissue sample;
c) identifying the subject as having a breast tumor that is CRYβB2 positive and may likely respond to a CDK4 inhibitor an anti-nucleolin agent or a ribosomal RNA synthesis inhibitor when the level of expression of CRYβB2 is greater than the level of expression of CRYβB2 in the reference sample; and d) administering to the subject an effective amount of a CDK4 inhibitor, an anti-nucleolin agent or a ribosomal RNA synthesis inhibitor.

12-13. (canceled)

14. The method of claim 11, wherein the breast cancer is estrogen receptor positive.

15. The method of claim 11, wherein the breast cancer is triple negative.

16. The method of claim 11, wherein the female subject is African American

17. The method of claim 11, wherein a CDK4 inhibitor is administered and is selected from the group consisting of palbociclib, ribociclib, and abemaciclib.

18. The method of claim 12, wherein a anti-nucleolin agent is administered and is an aptamer that binds nucleolin RNA

19. The method of claim 18, wherein the aptamer is AS 1411.

20. The method of claim 13, wherein the inhibitor of ribosome RNA synthesis is CX-5461 or BMH-21 and analogs thereof.

21. A method for treating breast cancer in a female subject comprising administering to the subject an effective amount of a CDK4 inhibitor, and/or an anti-nucleolin agent and/or a rRNA inhibitor when a sample of a plurality of cells from the breast cancer tissue of the subject is tested for nuclear and/or nucleolar expression of CRYβB2 protein and greater than 1% of the cell nuclei and/or nucleoli are positive for CRYβB2 protein.

22. The method of claim 21, wherein the breast cancer is estrogen receptor positive.

23. The method of claim 21, wherein the breast cancer is triple negative.

24. The method of claim 21, wherein the female subject is African American.
